(19) United States
(12) Patent Application Publication
(10) Pub. No.: US 2024/0016709 A1
(43) Pub. Date: Jan. 18, 2024

(54) BIODEGRADABLE MICROCAPSULES. PROCESS FOR PREPARING THE SAME AND METHOD OF USE THEREOF

(71) Applicant: ISP INVESTMENTS LLC, Wilmington, DE (US)

(72) Inventors: Madja AKRACH, Cenon (FR); Alastair VEITCH, Leeds (GB); Abdul Wahab HUSSAIN, West Yorkshire (GB); Alan FERNYHOUGH, West Yorkshire (GB); Karen MICTHINSON, West Yorkshire (GB); Claudia VENTURA, West Yorkshire (GB); Piero de LEONARDIS, Utrecht (NL)

(73) Assignee: ISP INVESTMENTS LLC, Wilmington, DE (US)

(21) Appl. No.: 18/015,689

(22) PCT Filed: Jul. 15, 2021
(86) PCT No.: PCT/IB2021/056420

§ 371 Date: Jan. 11, 2023

Related U.S. Application Data
(60) Provisional application No. 63/051,997, filed on Jul. 15, 2020.

(51) Int. Cl. A61K 8/11; C11D 17/00; C11D 3/00; C11B 9/00; A61K 8/85; A61Q 5/06; B01J 13/18; B01J 13/04

(52) U.S. Cl. CPC A61K 8/11; C11D 17/0039; C11D 3/001; C11B 9/0015; C11B 9/0011; A61K 8/85; A61Q 5/06; B01J 13/18; B01J 13/043; A61K 2800/10

(57) ABSTRACT

The present invention provides biodegradable microcapsules, that can encapsulate and retain cargoes such as, lipophilic or hydrophobic core materials comprising fragrances, butters, essential or other oils; or oil solubilized ingredients process of making said biodegradable microcapsules and their applications in various industries. Present invention further provides biodegradable shell materials that show evidence of biodegradation or non-persistence in aquatic based and/or soil or compost based environments.

### FIELD OF THE INVENTION

The present invention relates to biodegradable microcapsules, that can encapsulate and retain cargoes such as, lipophilic or hydrophobic core materials comprising fragrances, butters, essential or other oils; or oil solubilized ingredients, process of making said biodegradable microcapsules and their applications in various industries. Present invention further relates to biodegradable shell materials that show evidence of biodegradation or non-persistence in aquatic based and/or soil or compost based environments.

### BACKGROUND OF THE INVENTION

Conventionally, microcapsules (a) provide protection and stability to actives or ingredients entrapped inside the microcapsule; (b) facilitate, trigger, or control release of the entrapped actives or ingredients, (c) extend the life of the actives, (d) reduce the threat of exposures or (e) enable easily handling of entrapped actives which are otherwise toxic in nature or difficult to handle.

The microcapsules have an inner core material comprising lipophilic/hydrophobic compounds surrounded by an outer polymeric shell. The release rate of the core material and the diffusion of the core material through the capsule wall can often be controlled by varying the wall composition and/or the degree of crosslinking of the wall (shell) material. Further, the degree of crosslinking of the wall material directly impacts the strength and nature of the microcapsule wall.

A highly beneficial use, for example, of microcapsules is for the prolongation of fragrances, essential oils, or other lipophilic or oil solubilized ingredients which have been encapsulated inside a polymer shell. Typically, the technologies or materials used for encapsulation of fragrances or similar molecules (cargoes) have included melamine formaldehyde, urea-formaldehyde, or poly-urea/urethane technologies or acrylate technologies, most often using classical interfacial polymerizations. Because such cargoes are often aggressively solvating or plasticizing for many polymers and/or are volatile or have low boiling components difficult to retain within polymeric shells, such polymer shell walls used for their encapsulation are crosslinked networks of such polymers for stability and durability in the formulations in which they are used, for example, laundry/washing products, household cleaning products, hair care products skin care products among others. They are not designed to be biodegradable or non-persistent in the environments they may end up in. There are some known examples of biodegradable polymers used in microcapsule shell walls. They include polyesters or poly-β-amino-esters or poly-β-thio-esters, for example. However, in most cases, they are typically not encapsulating fragrances or essential oils or other solvating or plasticizing lipophilic or oil solubilized components for use in such consumer products, and/or are not biodegradable in relevant end media.

U.S. Pat. No. 8,287,849 (assigned to Massachusetts Institute of Technology) and scientific publication—“Degradable Poly-β-amino esters: Synthesis, Characterization, and Self-Assembly with Plasmid DNA” (written by Lynn, D. M, Langer, R.) published in J. Am. Chem. Soc. 2000, 122, 44, 10761-10768 disclose that poly-β-amino esters are typically unstable in solutions with wide pH range over hours to days, and are biodegradable in physiological or biomedical environments, comprising a compound prepared by reacting primary amine with bis-(acrylate ester).

U.S. Pat. No. 8,557,231 (assigned to Massachusetts Institute of Technology) describes poly-β-amino esters which are synthesized in organic solvents such as tetrahydrofuran (THF) or dichloromethane (DCM) and after isolation are then suggested as being useful in a complex double emulsion encapsulation process, typically also using a solvent (later to be removed) such as DCM, to make capsules for delivery of drugs or pharma based actives, for near term controlled release in biomedical physiological environments.

U.S. Pat. No. 8,945,622 B2 (assigned to Council of Scientific and Industrial Research CSIR) discloses a sustained release composition, useful for delivering active pharmaceutical agents comprising a graft polymer with a polyester backbone having the formula P [A(x)B(y)C(z)] prepared from diol (A), a dicarboxylic acid or acid anhydride (B) and a monomer (C) with pendant unsaturation onto which is grafted a polyacrylic or methacrylic acid chain. This patent describes tablet making processes.

US Patent Publication US2003/0224060 (assigned to L'Oréal) discloses nanocapsules having specific targeted cargo of retinoyl esters, which is described as a lipophilic active agent, and which have a water-insoluble envelope, comprising at least one polyester polyol wherein, this pre-made polyester polyol has been obtained by polycondensation of an aliphatic dicarboxylic acid or derivative with at least two alkane diols or with at least one alkane diol and at least one hydroxyalkyl alkane diol.

U.S. Patent Publication US 2007/0009441 (assigned to Molecular Therapeutics Inc.) discloses nanoparticle synthesis, their use in nanoscale (typically below 200 nm) encapsulations of water-soluble drugs or water-insoluble actives as solids for pharmaceutical applications. Biodegradability/biocompatibility in simulated physiological media was shown and in one aspect an itaconate polyester was used with specifically added crosslinkers for radical crosslinking, via aqueous radical initiator systems, with the itaconate polymer.

U.S. Patent Publication US2020/164332 (assigned to Calyxia SAS) describes a complex multi-layered microcapsule in which one polymer shell may contain esters but which is made by a very complicated double emulsion process and which uses radically polymerized monomer or polymers with added crosslinker.

European Patent Application EP 0517669 A1 (assigned to Sandoz) discloses process for microencapsulation of agrochemicals, obtained by microencapsulating an agrochemical in a crosslinked polymer capsule which is in part a polyester polymer, wherein such a process comprises the steps of (a) dissolving or suspending the agrochemical in a non-aqueous liquid mixture comprising unsaturated polyester resin and a vinyl monomer (preferentially styrene), (b) emulsifying said solution or suspension in water to a desired particle size; and (c) effecting crosslinking of the unsaturated polyester resin and vinyl monomer to produce the microcapsules.

PCT Publication WO2017125395 (assigned to BASF SE) discloses ‘biodegradable’ (in soil) polyester capsules comprising an aqueous core and a pesticide, wherein the capsule shell comprises a polyester, and the capsule core comprises a water-soluble pesticide (so a hydrophilic core), and at least 10 wt. % of water based on the total weight of the capsule core. Further, acid chlorides are used for its practical application to enable moderate temperatures and short reaction times for formation of the in-situ polyester in the presence of the cargo.

The Scientific publication—“Fragrance-containing microcapsules based on interfacial thiol-ene polymerization” (by Liao et al) published in J. Appl. Polym. Sci. 2016, 133, 43905 doi: 10.1002/App.43905, discloses fragrance capsules with a poly-β-thio ester shell wall, made using a classical interfacial polymerization route.

Such prior art have numerous disadvantages wherein: (i) some are for water soluble actives and so not suitable for hydrophobic or lipophilic materials; (ii) few show or claim or are designed for biodegradability, none show or claim biodegradability in ambient aquatic environments or in related OECD tests, (iii) many use organic solvents to enable encapsulation which are problematic in removal and for use with volatile cargoes, (iv) some make the polymer shell wall itself in the presence of the cargo via interfacial polymerizations where one reactive or catalytic ingredient is in the water phase and another in the oil phase and where, as such, they necessarily use (in the case of polyesters) undesirable acid chlorides to maintain low reaction temperatures and hence emulsion stability and comparatively short reaction times (higher temperatures, as required for diacid-diol condensation reactions would typically destabilize the emulsion and lead to unsuccessful encapsulations and lead to loss of components of fragrances or similar cargoes), or (in the case of β-thio-esters) necessarily require a large excess of an undesirable reactant (such as a water soluble, odorous, thiol), and/or require the use of undesirable solvents and their evaporation, and (v) few, if any, show any ability to be stable on storage as made with fragrance or oils or other plasticizing cargoes inside, or show stability or utility on storage in end product formulations as are used in home or personal care applications which may have pH extremes or surfactants or salts or solvents or other additives that may plasticize or attack the shell wall.

There remains a significant challenge to encapsulate lipophilic or hydrophobic cargoes in a polymeric shell which can biodegrade in aquatic or other media and which is robust enough to hold the cargo (often in aqueous based formulations of personal care or household or other products) until release is triggered or required and/or to release it in a gradual process or controlled way.

Current inventors aim to meet these criteria and so enable production of microcapsules that have a shell material that is biodegradable or non-persistent, particularly in aquatic media/waterways, and yet which can retain a hydrophobic or lipophilic cargo or a volatile or a plasticizing or oil solubilized cargo such as a fragrance or an essential oil or other oil, and which is stable on storage in a product form until use.

### SUMMARY OF THE INVENTION

We have surprisingly discovered that volatile or plasticizing hydrophobic or lipophilic ingredients such as fragrances, oils and other lipophilic cargoes can be encapsulated within robust storage stable polymer shells incorporating aliphatic ester and/or β-amino-ester and/or β-thio-ester moieties in their backbones and/or in their branches and/or in their crosslinks and, furthermore, in addition, that through the associated polymer shell precursors and polymer architectures, such as linear, branched, crystalline or crosslinked polymers, such polymer shell systems can meet important biodegradability criteria and in particular such criteria for biodegradability or non-persistence in ambient aquatic environments such as seawater, river/surface water, effluents, and/or other water treatment process streams (e.g. activated sludge).

In one important aspect, the present application encompasses microcapsules based on polymeric shell walls with esters, and/or β-amino esters and/or β-thio esters.

Accordingly, the present application provides a microcapsule comprising: (i) a lipophilic core; and (ii) a polymeric microcapsule shell; wherein, the polymeric microcapsule shell comprises a polymer or a crosslinked polymer of an aliphatic polyester or a poly-β-amino-ester or a poly-β-thio-ester or their co-polymers or ter-polymers or mixtures thereof; wherein, the microcapsule is storage stable and its polymeric shell is biodegradable.

Yet another aspect of the present application is to provide methods for preparing said microcapsules. Accordingly, the present application provides a method for preparing microcapsule, the method comprising: (a) preparing an oil-in-water emulsion of (i) an oil phase comprising a polymer or a prepolymer, and at least one lipophilic core; and (ii) a water phase comprising at least one stabilizer or emulsifier, (b) optionally adding at least one catalyst, at least diluent or at least one initiator to the oil phase, (c) optionally heating the oil-in-water emulsion with stirring to a temperature between 25° C. and 100° C.; (d) forming the polymeric microcapsule shell either by cooling or by an in-situ oil in water reaction of the polymer or prepolymer, and (e) obtaining the core encapsulated in a polymeric microcapsule shell; wherein, the formed polymer or prepolymer is an aliphatic polyester or a poly-β-amino ester or a poly-β-thio ester or their co-polymers or ter-polymers or combinations thereof.

Accordingly, the present application provides a method for preparing microcapsules, the method comprising: (a) preparing an oil-in-water emulsion of (i) an oil phase comprising monomeric reactants and at least one lipophilic core; and (ii) a water phase comprising at least one stabilizer or emulsifier, (b) optionally adding at least one catalyst, diluent or at least one initiator to the oil phase or water phase, (c) forming the polymeric microcapsule shell wall by an in-situ oil-in-water polymerization reaction of the monomeric reactants, and (d) obtaining the core encapsulated in a polymeric microcapsule shell.

Accordingly, the present application provides a method for preparing microcapsules, the method comprising: a) making an oil-in-water emulsion of an oil phase which comprises a difunctional or multifunctional-acid and a diol or multifunctional alcohol, a cargo, optionally with added diluent or solvent and/or aided by application of heat, and a water phase containing a stabilizer and/or other additives, b) adding a catalyst to one phase, c) forming the polymeric capsule shell wall by an in-situ oil-in-water polycondensation (esterification) polymerization reaction of the monomeric reactants or other precursors and d) obtaining the cargo encapsulated in a polymeric microcapsule shell.

Accordingly, the present application provides a method for preparing microcapsules consisting of β-thio ester and β-amino ester functionalities, the method comprising: (a) pre-reacting a difunctional or multifunctional amine with difunctional or multifunctional acrylate; (b) preparing an oil-in-water emulsion of (i) an oil phase comprising the resultant or product of (a) and any remaining acceptor, mixed with a difunctional or multi-functional thiol, and at least one lipophilic core, optionally with a diluent, and (ii) a water phase comprising at least one stabilizer or emulsifier; (c) optionally adding at least one catalyst to the oil phase or water phase, (d) forming the polymeric microcapsule shell wall by an in-situ oil-in-water Michael addition polymerization reaction of the donor and acceptor reactants, and (e) obtaining the lipophilic core encapsulated in a polymeric microcapsule shell.

### BRIEF DESCRIPTION OF THE DRAWINGS

Further embodiments of the present application can be understood with the appended figures.

FIG. 1 represents aliphatic polyester microcapsules (201-13-1 0.95 SA/0.05 IA/1.00 HD) of the invention with 25 wt % fragrance encapsulated: (i, left) pre-application of pressure or rubbing: under microscope slide (ii, right) post application of pressure or rubbing showing cargo release.

FIG. 2 represents dried aliphatic polyester microcapsules (201-13-1) of the invention with ˜25 wt. % fragrance loading: (i, left) dry, under a microscope slide; (ii, right) the same dried capsules re-dispersed in water and crushed to release cargo under a microscope slide.

FIG. 3 represents optical micrographs of aliphatic polyester microcapsules of the invention: capsules made with PLGA prepolymer/polymer—before and after crushing (Example 14).

FIG. 4 represents images from optical microscopy for microcapsules of Example 17 (210-26-1)—before and after crushing under a microscope cover-slip to show fragrance release (capsules made via in-situ Michael Addition Polymerization).

FIG. 5 represents images from optical microscopy for microcapsules of Example 18 (210-86-1)—before and after crushing under a microscope cover-slip to show fragrance release (capsules made via in-situ Michael Addition Polymerization).

FIG. 6 represents images from optical microscopy for microcapsules of Example 19 (210-91-1)—before and after crushing under a microscope cover-slip to show fragrance release (capsules made via in-situ Michael Addition Polymerization).

FIG. 7 represents images from optical microscopy for microcapsules of Example 20 (210-82-1) before and after crushing under a microscope cover-slip to show fragrance release (capsules made via in-situ Michael Addition Polymerization).

FIG. 8 represents images from optical microscopy for microcapsules of Example 22 (215-52-1) with isophorone diamine and hexathiol as donors with a tetra-acrylate acceptor before and after crushing under a microscope cover-slip to show fragrance release.

FIG. 9 represents images from optical microscopy for microcapsules of Example 22 (215-42-1) with hexamethylene diamine and hexathiol as donors with a tetra-acrylate acceptor before and after crushing under a microscope cover-slip to show fragrance release.

FIG. 10 represents images from optical microscopy for microcapsules of an example (215-55-1) with TMPP diamine and a trithiol (trimethylolpropane tris-(3-mercaptopropionate) as donors before and after crushing under a microscope cover-slip to show fragrance release.

FIG. 11 represents optical microscopy images of spray dried capsules of Example 25 (210-48-1) (capsules made by Michael Addition polymerization), before and after crushing to show fragrance release.

FIG. 12 represents optical microscopy images of microcapsules (215-42-1) prepared from Butanediol diacrylate, 4,4 Trimethylene dipiperidine and Pentaerythritol hexakis (3-mercaptopropionate) with fragrance Sunburst fresh R14-3913.

FIG. 13 represents sensory test results for fragrance release from polyester microcapsules of the invention referenced to a fragrance-only sample (R14-3913).

FIG. 14 represents sensory test for fragrance release from microcapsules of the invention (capsules made by Michael Addition polymerization)

FIG. 15 represents sensory test results for fragrance release from further microcapsules of the invention (capsules made by Michael Addition polymerization)

FIG. 16 represents sensory test results for fragrance release from microcapsules (made by Michael Addition polymerization), of the invention (referenced to fabric conditioner without fragrance (A) and with neat fragrance (B).

FIG. 17 represents controlling biodegradation of microcapsule shell materials via cross-linking/branching/chain extension of polyester prepolymers with reactive unsaturation with/without Vazo 67 (V67) as radical initiator

FIG. 18 represents biodegradation data for microcapsule shell materials made via an in-situ oil-in-water Michael Addition polymerization with DCM as cargo, subsequently evaporated. Data shows gradual, positive, ongoing biodegradation for various compositions of β-thio esters and β-amino-co-β thio esters to 40 days.

### DETAILED DESCRIPTION OF THE INVENTION

For physical-mechanical encapsulation methods, such as spray drying, particle size control is generally achieved through control of the physical conditions under which the involved processes are carried out. Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “core” and “cargo” as used throughout the specification are inclusive and refer to same ingredient forming part of the microcapsule encapsulation.

The term “each independently selected from the group consisting of” means when a group appears more than once in a structure, that group may be selected independently each time it appears.

The term “polymer” as used herein, refers to a compound comprising repeating structural units (monomers) connected by covalent chemical bonds. Polymers may be further derivatized, crosslinked, grafted, branched, or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “pre-polymer” as used herein, refers to any polymer or oligomer pre-made prior to the encapsulation process stage, and which can undergo some form of physical or chemical transformation during or after the process of encapsulation such as a reaction (chain extension, branching, molecular rearrangement, crosslinking, ionic or other linking or molecular association) or crystallization.

Monomeric reactants are defined as reactants which are small molecules, and which can react together to build up a polymeric structure. They may be difunctional or have higher functionality, or be multifunctional or poly-functional, or, if self-polymerizing, they may be mono-functional.

The release rate of the core material and the diffusion of the core material through the capsule wall can, in many cases, be controlled by varying the wall composition and/or the degree of crosslinking of the wall (shell) material. Also, the degree of crosslinking of the wall material directly impacts the strength and nature of the wall of the microcapsule. Furthermore, if a material is encapsulated, its useful life can be significantly extended. Also, if a material is toxic and/or difficult to handle, encapsulation of the material can reduce the threat of exposures and/or allow for easier handling.

Fragrances and oils and other lipophilic ingredients are widely used in personal and household care products such as detergents, fabric softeners, shampoos, and shower gels to enhance the product performance and attributes. Long lasting release of fragrances is a key performance parameter in many personal and household care products, yet many fragrances or oils are volatile, and their aroma effects are quickly lost on application. Encapsulation of fragrances inside a solid shell can protect fragrances and enable longer lasting release. Among the existing encapsulation systems, polymeric microcapsules made via interfacial polymerizations are widely used. These can be via oil-in-water (O/W) or water-in-oil (W/O) emulsions wherein typically, monomers react at the oil-water interface to form a polymeric shell. The majority of commercial fragrance microcapsules consists of poly(urea-formaldehyde), poly(melamine-formaldehyde), polyurethane, or polyurethane-urea shell materials or polyacrylates. Example References: U.S. Pat. 20080206291; WO2013092375; U.S. Pat. 20130337023; Chem. Eng. J. 2009, 149, 463; and WO 2017123965A1 are incorporated herein in its entirety.

These particular systems such as M-F systems, or U-F (urea-formaldehyde) or radically crosslinked acrylate or crosslinked urea or urethane systems, are chosen for their superior thermal and mechanical properties and are all rigid or highly crosslinked systems in order to retain volatile ingredients or ingredients that have a tendency to plasticize or dissolve away other shell walls or leach out through shell walls of other systems and also chosen for their stability in a wide range of end product formulations. They typically use low viscosity reactive monomers or reagents to enable interfacial or in-situ polymerization-encapsulation processes to proceed smoothly to form rigid, highly insoluble, and highly crosslinked polymer systems. They are designed for durability, including long lasting stability in various formulations over a range of pH's with salts or surfactants or solvents or other additives present which may compromise some other polymer shell walls, and also have the feature of being friable meaning they are able to be crumbled or broken by application of pressure or friction such as in service or when required for release. This, for example is an attractive attribute in fragrance encapsulations for formulations or applications where a fragrance bloom (instant but long lasting or repeatable over time, release of cargo) can occur when shell walls are ‘crumbled’ or broken in service for example by rubbing or friction. Such durable polymeric shell materials are not claimed to be, nor would they be anticipated to be, biodegradable in aquatic environments, or indeed other environments such as soil or compost. Typically, such highly crosslinked or rigid polymer particles or capsules would be expected to be persistent or very slowly degrading in the environment and/or often use environmentally toxic or unfriendly materials such as formaldehyde or isocyanates in their production. Other routes such as coacervation do not make as robust a capsule and/or may require the use added undesirable solvents or use undesirable animal derived ingredients. There is a need for a robust microcapsule that can encapsulate, and retain until a triggered release, volatile or plasticizing cargoes and be biodegradable in the environments in which they end up in, which in many cases will be aquatic systems such as rivers, oceans or water treatment plants.

We have surprisingly discovered that volatile or plasticizing hydrophobic or lipophilic ingredients such as fragrances, oils and other lipophilic cargoes can be encapsulated within robust storage stable polymer shells which are biodegradable and yet which can display a similar or acceptable degree of cargo retention and bloom (release) to some non-biodegradable robust polymer shell options, and can in some cases also show longer lasting release of cargoes such as fragrances or oils or other lipophilic cargoes. Such polymer shells can be made using linear polymers or branched polymers, optionally with high Tg or crystalline domains, or using lightly crosslinked or, highly crosslinked polymer systems incorporating ester and/or β-amino-ester and/or β-thio-ester moieties in their backbones and/or in their branches and/or in their crosslinks. Furthermore, in addition to successful encapsulation and retention of potentially plasticizing or solvating lipophilic cargoes we have discovered that by selection of the polymer shell precursors and polymer architectures, such as linear or branched or crystalline or crosslinked polymer shell systems, such polymer shell materials can meet important biodegradability criteria and in particular such criteria for biodegradability or non-persistence in ambient aquatic environments such as seawater, river/surface water, effluents, and other water treatment process streams (e.g. activated sludge). Furthermore we have found that in some such combinations or systems the biodegradable capsules of our invention are also able to be stored ‘as is’ (as-made) or in formulated end products of various types and over a range of pH's and with common formulating additives such as surfactants or solvents or salts etc. also present. Thus, the invention encompasses a suite of microcapsule compositions, based on polymeric shell walls with the esters, and/or β-amino esters and/or β-thio esters mentioned, all of which can be designed to be biodegradable according to criteria herein described, and all of which have encapsulated lipophilic cargoes and are able to be tailored to meet the difficult combination of biodegradability, storage stability in formulated products, and triggered performance release or bloom of cargo, which span a range of performance levels suited to different formulated end products or applications and/or different encapsulated cargoes for those end product formulations. In addition, the invention also encompasses a suite of processes for the production of said microcapsules.

The capsules of the invention are also able to be dried and stored dried and subsequently redispersed into formulations. They may also be formulated, directly as a slurry or after drying, into dry or ‘waterless’ formulated product forms such as tablets or soap bars or printed solid products other solid formats in various end applications, particularly, but not limited to, those used in personal and home care markets.

Biodegradation and non-persistence of materials which are in the environment are influenced by multiple factors. These include factors such as: (a) the environment in which the material finds itself either in use and/or after use, and the many factors therein such as, temperatures, humidity/water presence, pH, microbial populations, nutrients, etc., and (b) the timescale for monitoring or predicting biodegradation. Material composition, structure, morphology and physical size and form are also important factors, for a material of interest.

Evidence of biodegradation or non-persistence may be achieved via demonstrating a certain level of degradation within a time and/or degradation at a rate that is indicative that ultimately the material will degrade and be non-persistent after a certain time. There are many testing standards or methods which specify certain tests and associated timescales. In use there are some applications which may desire certain levels of biodegradation within certain timescales and environments to be met for evidence of biodegradation. Of course, for some applications a certain level or % of biodegradation may be desirable or even stipulated as evidence of more rapid biodegradation or of a certain minimum level of biodegradation. Others may specify evidence of non-persistence. As to what evidence of biodegradation and/or of non-persistence is required or desired depends on the details of the end-application, the expectations of the customers (often the final product manufacturers) or of the consumers (typically, the end-users) and can vary according to end use and end environments or regulatory body directives or guidelines, of which there may be many variations. OECD, ISO, ASTM, EN or other standards for testing are commonly used to measure biodegradation or compostability and can be used as evidence of non-persistence or, indeed, conversely, of any likely persistence in an environment. However, they are not the only methods available and many publications report on other methods or criteria, and which are peer reviewed or rational to those skilled in the art. Furthermore, different end use sectors (products) and different regions of the world have different specifications or guidelines such that there is no single universal definition.

In the case of polymers, biodegradation typically begins with breakdown of the polymer chains or backbone into smaller components which continues until they become small enough to be intracellularly metabolized by micro-organisms such as bacteria, yeast, or fungi. Often the first steps of initial breakdown of polymers proceeds via hydrolysis or oxidation of the polymer backbone chains to generate smaller molecules suitable for intracellular consumption. Hydrolysis is a particularly common first step and may be facilitated, for example, by secreted extracellular enzymes (enzymatic hydrolysis; secreted by microorganisms in the end- or test-environment) and/or by the certain ambient conditions (pH, temperature, etc.).

Many polymers are resistant to biodegradation and persist in the environment for years or decades, for example many plastics, which are often used in applications for their long lasting durability. Similarly, many particles or microplastics are known to persist in the environments they end up in. This includes the many of the microcapsules of the prior art such as those based on M-F, U-F, crosslinked urea or urethane, and crosslinked polyacrylates. This has become a concern for the global environment such that nations and organizations, such as ECHA, may implement bans or restrictions on the use of microplastics that persist in the environment in certain products. In some respects, depending on the materials used, and their characteristics, microcapsules may be considered as a form of microplastics. As noted above microcapsules are very convenient for protection of cargoes (entrapped actives or ingredients) and/or controlled release of cargoes. Thus, biodegradable microcapsules are sought after.

Biodegradable capsules are known and particularly in the fields of biomedical and pharma applications. Common polymers include polyesters among others. Biodegradation in such applications is in physiological human (or animal) body environments and typically are at 37° C. and often with extremes of pH and/or a high presence of enzymes or nutrients that specifically facilitate breakdown of such polymers. Typically, the cargoes are solids, or water-soluble actives, and/or do not have volatile or reactive components. Also routes to manufacture such capsules involve undesirable solvents (such as dichloromethane) and/or processes such as microfluidics or freeze drying or evaporative processes, or extrusion methods, which are all impractical technically and/or commercially for encapsulating volatile fragrances and similar lipophilic cargoes or for applications in cosmetics or the personal care and household sector. Further, biodegradability in such biomedical/pharma environments (with their higher temperatures, enzyme presence, and more aggressive (for degradation) conditions etc.) is not indicative of, or comparable to, biodegradability in ambient aquatic waterways or seawater for example and, also, not reflective of the needs of the personal care or household sector, and other sectors (e.g. drilling/energy), where many of the products used will end in aquatic environments such as rivers, seas, surface water, water treatment plants/effluents—which are essentially ambient temperature (20° C. or lower) waters, or in soils or sediments.

It is reported that some of the microcapsules or other ingredients which are used in the personal care and household sector today may potentially be considered persistent in the environment and that they may fall under the umbrella definition of microplastics, and as such are undesirable. All such products as may be classed as microplastics are likely to be restricted in their use in personal care and household, and other products at some stage in the future. ECHA has initiated proposed processes for that. Other bodies may develop similar or alternative guidelines or protocols. Thus, there is a need to develop polymer capsules that are biodegradable in environments where common personal care and household products may eventually end up in. Demonstrating reasonable biodegradability of an ingredient will likely such avoid restrictions assuming other factors are also favorable. OECD and ISO test methods are typically specified for biodegradation testing in some cases. Other test standards are also used and are likely to be relevant and including future new standards as may be developed or specified. Thus, there is a need to develop polymer capsules that are biodegradable in environments where common personal care and household products, and many other products, may eventually end up in, and which can be manufactured in commercially sensible processes for that sector (so not using solvents requiring evaporation or high temperature encapsulation processes, for example).

In some OECD biodegradation tests for aquatic media, which are typically in relatively short timescales such as 28 days, under certain test conditions, achieving 60% biodegradation within 28 days in certain OECD tests can lead to a classification of being readily biodegradable. Such a material would be considered as rapidly biodegradable. In some OECD tests achieving 20% biodegradation can indicate a classification of a material being inherently biodegradable or primary inherently biodegradable. This indicates the potential for a material to be biodegradable, which would be over longer timescales than for readily biodegradable materials. Although 28 day tests are the standardized duration in some OECD tests, for the inherent classifications, extended testing periods of up to 60 days and longer if biodegradation has started within 28 days and has not yet reached plateau (see for example Annex 1 of OECD document OECD Guideline for Testing of Chemicals: part 1 Principles and strategies related to the testing of degradation of organic chemicals available at https://www.oecd.org/chemicalsafety/testing/34898616.pdf, in particular paragraphs 21 and 36).

Thus for the purposes of this invention, evidence or data for biodegradability or evidence of non-persistence is tested in aquatic environments or media such as activated sludge, secondary effluent, river- or surface- or sea-water and the like according po OECD test standards but may be for longer than 28 days when biodegradation has started and not reach a plateau. Typically testing of biodegradation herein is according to methods of OECD or ISO test protocols, such methods and their variants as described for OECD 301, 302, 306, 310 or EN ISO 14852:2018 or EN IS014851:2004 or EN ISO 19679:2016 or EN ISO 18830:2006 or EN ISO 17556:2012) or analogous or other standards. If in using such tests, about 20% biodegradation has been attained within 28 days or, is attained within a longer time period if biodegradation has started within 28 days and not reached a plateau, then that is provided as evidence for being biodegradable or non-persistent. Such evidence for biodegradation can be demonstrated within 28 days or 40 days, or 45 days or 60 days or 90 days or 3 months, or within 6 months, or within 12 months, or longer when tested according to standards, if no plateau is evident. Preferably for the purposes of this document 20% biodegradation will have been obtained within 60 days of such a standard OECD aquatic media and not shown a plateau in the biodegradation vs time plot. Thus, for testing purposes here, evidence or data for being biodegradable means evidence for inherently biodegradable or inherently primary biodegradable as per the OECD test methods and descriptions, including within longer timescale where allowed for in order to achieve 20% biodegradation with no plateau. It should be noted that not achieving such levels is not indicative of persistence—other tests can be applied to demonstrate non persistence or biodegradability in aquatic or other media. In addition, such OECD aquatic tests are typically at ambient conditions (20-25° C. or lower) and it will be recognized that biodegradability in other media (compost, soil, and sediments) will also be likely attainable if biodegradation in aquatic media is demonstrated. Also, ready biodegradability is also covered should it be demonstrated. Furthermore, OECD methods are not the only relevant test methods, although in this document they have been used for test data. Other criteria can be accepted and are used by others and in certain regions or applications. Other standard test methods or justifiable variations can be used, and other data may be accepted by industry regulators or by experts or if showing a sensible or logical rationale and/or where other evidence of non-persistence may be presented and accepted by those skilled in the art. For example, molecular weight reductions or weight loss or other measurements as evidence of biodegradation or non-persistence particularly for more slowly degrading materials may be used. Degradation half-life determinations are also be used. All are potentially relevant depending on the circumstances. This document reports biodegradation data using OECD test methods, though it is recognized such other tests or criteria may also be applied in to show biodegradation or non-persistence. In particular for the many samples or material types which are insoluble in water, dispersions or films, other approaches are used to achieve reliable sample forms for biodegradation tests. It is recognized that today's test methods (OECD or other) for biodegradation of polymers in aquatic media are not necessarily representative, having not being designed or intended for testing such materials when originally conceived and especially for water-insoluble polymers and also that refined or improved or alternative test methods may in time be developed which are likely to be more relevant. (See for example: Kowalczyk, A. et al (2015) Refinement of biodegradation tests methodologies and the proposed utility of new microbial ecology techniques. Ecotoxicology and Environmental Safety, 111, 9-22. https://doi.org/10.1016/J.ECOENV.2014.09.021 and: Timothy J. Martin, et al (2017) Environmentally Relevant Inoculum Concentrations Improve the Reliability of Persistent Assessments in Biodegradation Screening Tests. Environ. Sci. Technol. 2017, 51, 3065-3073, DOI: 10.1021/acs.est.6b05717). It would be expected that if a material is showing evidence of biodegradation or potential non-persistence in the OECD or ISO tests reported in this document then it will also be biodegradable or non-persistent in future test specifications, likely more suited to polymers and the environments of today or the future. Also, where testing of polymers in seawater (marine), or surface/river water or activated sludge has shown some, even low levels of, ongoing biodegradability then such materials will likely show greater rates or degrees of biodegradation in more active media such as soil or compost, or other in media where enzymes or microorganisms are present in greater concentrations or diversity. It is reasonable to assume, and generally understood, that if biodegradability is shown in the usual aquatic media tests for a material, then the material would also be expected to be compostable according to the various standard tests for compostability. Furthermore, and similarly, it would also be reasonable to assume biodegradability in soil or similar media if shown to biodegradable in aquatic media. The reverse, however, is not able to be stated. Thus, if a material is confirmed as compostable, it is understood that it is not an indication that it will degrade in waterways or other ambient aquatic media. Polylactic acid is a well-known example of a polymer (polyester) that is compostable but will not biodegrade in aquatic media or soil. Thus, the testing in this invention is based on aquatic media on the basis that if a material is showing biodegradability in ambient aquatic media, it will also be compostable and degradable soil, according to typical standard test methods.

Thus in summary, it will be understood by those skilled in the art that for biodegradation testing the testing of biodegradability in aquatic media such as surface water or secondary effluent or activated sludge, as described by the aforementioned OECD tests, all carried out a temperatures around 20-25° C., is relatively mild and certainly less aggressive as a test for biodegradability compared to, for example, biodegradation testing in industrial composting facilities and via the test methods or standards developed for compostability testing such as EN13243 or ASTM D-6400 or ASTM D-6868, and others. In such compostability testing usual temperatures are much higher, for example around 58-60° C. It is understood that many polymers including polyesters such as polylactic acid which do show biodegradability in industrial composting tests are not able to show biodegradability in aquatic media tests (see for example: Bagheri, A. R., Laforsch, C., Greiner, A., Agarwal, S.: Global Challenges 2017, 1700048; DOI:10.1002/gch2.201700048). However, polyesters, or indeed other polymers, which do show evidence of biodegradation in such aquatic OECD tests would be confidently expected to be also compostable and able to pass tests for compostability.

A highly beneficial use, for example, of microcapsules is for the prolongation of fragrances or other ingredients which have been encapsulated inside a polymer shell. Typically, the technologies or materials used for encapsulation of fragrances or similar molecules (cargoes) have included melamine formaldehyde pool urea/urethane technologies or acrylate technologies. Most use crosslinked networks of these polymers for stability and durability in the formulations in which they are used (for example, laundry/washing products, household cleaning products, hair care products skin care products among others.)

Thus, making microcapsules able to contain hydrophobic or lipophilic groups which may, also, optionally, be volatile and/or plasticizing, requires some alternative approaches to what is known in the prior art for making microcapsules suitable to encapsulate lipophilic or hydrophobic cargoes and yet which can also be biodegradable and especially biodegradable in aquatic environments such as seawater, rivers, surface water or in water treatment effluents, processes, or activated sludges.

It is an aim of this invention to meet these criteria and so enable production of microcapsules that have a shell material that is biodegradable or non-persistent, particularly in aquatic media/waterways, and yet which can retain a hydrophobic or lipophilic cargo or a volatile or a plasticizing or oil solubilized cargo such as a fragrance, an essential oil or any other oil, and is stable on storage in a product form until use. Fragrances are of prime interest since they are used in many end products and yet they typically have some volatile or low boiling components which can evaporate quickly if not contained in some way and/or components which are plasticizing to many polymers.

In terms of prior art there are many patents and publications on microencapsulation of actives which are lipophilic. There are many examples of microencapsulations, of hydrophilic and lipophilic components for pharmaceutical or biomedical applications which describe biodegradable shells for controlled release. Biodegradation in such physiological environments are not representative of biodegradation requirements in aquatic waterways and the like. Physiological environments are typically warm at 37° C., have mixtures of specific degrading enzymes do not present in aquatic waterways for example, and/or have local pH extremes, and/or have salts and many other chemical entities also present. Overall, they are relatively aggressive media for degradation for controlled release. Furthermore, the shell wall materials, many of which are polyesters, and/or the processes typically used in drug or pharma active delivery are typically not suited to volatile or plasticizing cargoes. Many processes use extrusion (high temperatures), or solvents (requiring evaporation to very low residual limits) and when they do use undesirable components or reactants for shell walls (e.g. isocyanates for urethane shells) they will require significant cleaning or work-up to ensure removal of trace amounts of such components. Many pharma based encapsulations using polylactide or polyglycolide or poly(glycolide-co-lactide) (PLGA) polyesters as capsule shells use, for example, dichloromethane as an enabling solvent for encapsulations and it is necessarily subsequently removed by evaporation. All such aspects are not suited to volatile or plasticizing cargoes and/or are prohibitively expensive in their work up or other process stages for applications outside of pharma. For developments that are biodegradable shells of capsules, excepting those for pharmaceutical or biomedical applications, where, as just described, end environmental conditions (pH, temperature and/or presence of special enzymes etc.) are quite different from those in waterways and soil and where processes for manufacture are not well suited to those in personal or home markets, there are fewer in number and all of which have drawbacks inhibiting their widespread practical applicability. Our invention overcomes such drawbacks while also meeting the criteria described stated above.

In the existing prior art claiming polyesters for encapsulation, some are for water soluble actives and so not suitable for hydrophobic or lipophilic materials; few show or claim biodegradability, none show or claim biodegradability in ambient aquatic environments or in related OECD tests, many use organic solvents to enable encapsulation which are problematic in removal and for use with volatile cargoes, some make the polyester itself in-situ in the presence of the cargo via interfacial polymerizations where one reactive or catalytic ingredient is in the water phase and another in the oil phase and where, as such, they necessarily use undesirable acid chlorides to maintain low reaction temperatures and hence emulsion stability and comparatively short reaction times (higher temperatures, as required for diacid-diol condensation reactions would typically destabilize the emulsion and lead to unsuccessful encapsulations and lead to loss of components of fragrances or similar cargoes) and/or require the use of undesirable solvents and their evaporation. None claim or show a combination of such biodegradation properties with a successful encapsulation of a fragrance or similar volatile lipophilic cargo with the attributes of imparting a noticeable bloom or release of cargo when triggered (e.g. when rubbed or application of pressure). Furthermore, few if any show any ability to be stable on storage as made with fragrance or oils or other plasticizing cargoes inside, or show stability on storage in end product formulations as are used in home or personal care applications which may have pH extremes or surfactants or salts or solvents or other additives that may plasticize or attack the shell wall.

In one embodiment, the present application provides a microcapsule comprising: (i) a lipophilic core; and (ii) a polymeric microcapsule shell; wherein, the polymeric microcapsule shell comprises a polymer or a crosslinked polymer of an aliphatic polyester or a poly-β-amino-ester or a poly-β-thio-ester or their co-polymers or ter-polymers or mixtures thereof; wherein, the microcapsule is storage stable and its polymeric shell is biodegradable. The polyester based capsules of our invention show successful microencapsulation and subsequent triggered release of fragrance or other lipophilic cargoes with associated evidence for biodegradability or potential non-persistence, in aquatic media according to OECD test methods and made via convenient processes at low to moderate temperatures suited to volatile ingredient encapsulations, and not requiring subsequent volatile solvent removal or the use of undesirable isocyanate or acid chlorides or the use of high temperatures at the encapsulation stage.

Poly-β-amino and poly-β-thio ester homopolymer complexes, particles and capsules have also been described. The polymers are typically made by Michael Addition, or conjugate addition, reactions of a difunctional or multifunctional donor (e.g. an amine (primary or secondary), Aza-Michael) or a thiol (Thio-Michael)) with a difunctional or multifunctional acceptor (e.g. an activated (electron deficient) conjugated double bond as in an acrylate or related molecules, well known in the field). Solvent based methods are applied, e.g. with water as a solvent, typically making hydrogel based encapsulations from such precursors. Other solvent mediated processes, or classical interfacial polymerizations (oil-in-water wherein the donor is one phase and the acceptor and/or a catalyst is in the other so, requiring a water soluble reactant) have also been applied, though less frequently, to make capsules from these polymers and the precursor Michael Addition reagents. These typical methods have disadvantages when trying to encapsulate polar or volatile or plasticizing cargoes in a robust, highly crosslinked or rigid shell.

When a water-soluble donor is reacted with a water-soluble acceptor in water typically hydrogel matrix capsules result. These are suited to controlled release over time of drugs, for example. Hydrogel matrix capsules are generally are not as retentive or robust as core shell capsules and, as made, are not well suited to encapsulation of ingredients or actives (such as a fragrance for example) where a triggered more instant release of cargo is desired. Also, they would not be suitable for storage in aqueous based formulated products requiring long term retention before release of their cargo. When solvent mediated processes are used for encapsulations with such reagents the solvents will typically need to be removed (typically evaporation and/or other complicated double emulsion or other processes will be required), and, as described above, these are not well suited to volatile cargoes or to commercially viable processes for personal or home care applications. In addition, much of the prior art describes poly-β-amino esters for biomedical/pharma environments and as useful in these media for their very rapid degradation—good for biodegradability profiles but not well suited to storage (until required use) in aqueous formulations as used in personal or home care applications.

Similar comments apply to poly β-thio esters: hydrogels can be made but have similar disadvantages as just described. Also, again, publications are related to pharma or biomedical applications. One example outside of that field does report on a classical interfacial polymerization for synthesis of fragrance capsules. (See: Liao et al, Fragrance-containing microcapsules based on interfacial thiol-ene polymerization. J. Appl. Polym. Sci. 2016, doi: 10.1002/App.43905). The publication does not suggest or measure any biodegradation performance nor design the capsules for any biodegradability response. Furthermore, in such classical interfacial oil-in-water polymerizations (and encapsulations) typically an oil soluble acceptor (e.g. a difunctional acrylate if an acceptor) or donor is reacted, with a water-soluble donor (e.g., a difunctional amine or thiol if a donor) or acceptor. Interfacial polymerizations with such systems proceed typically with the cargo and one monomer (acceptor here) in an oil phase and emulsified with a water-surfactant mixture to make a pre-emulsion. Subsequently, a second monomer (thiol donor here), which is necessarily water soluble is mixed with the pre-made emulsion and the interfacial polymerization reaction proceeds forming a shell around the cargo at the interface. Such interfacial polymerisations have disadvantages for such encapsulations of some cargoes, including those that are more polar or plasticising or volatile molecules, and including many natural or essential oils or fragrances or other hydrophobic or lipophilic cargoes, for several reasons; first, an excess of donor (e.g. amine or thiol) tends to be required, leading to, for such interfacial polymerizations, the presence of residual (unreacted) monomers and so requiring more rigorous washing or clean-up processes at the end of reaction. This is highly inconvenient, wasteful and expensive for commercial processes since very low residual amounts of such reactants for personal or home care products will typically be required.

Furthermore, in the case of excess thiols being used as descried here it can also lead to unpleasant odour concerns due to residual unreacted thiol—as the case in this referenced document, where a 50% mole excess of a such water soluble thiol (1.5 SH to 1 acrylate bonds) was stated as being required so leading to high levels of residual thiol and an associated odour. Second, donors such as water soluble amines or water soluble dithiols are relatively hydrophilic, and therefore the resulting polymers are more swellable or softer in water or polar solvents and so potentially more leaky when stored in aqueous media (as made (slurries) for example) or when formulated in aqueous media, as is common in many applications in laundry or home care or personal care for example. This can negatively impact cargo retention (e.g. fragrance or oil or other hydrophobic cargo) and/or storage stability across a wide range of pH's retention especially where release of cargo is not sought to be gradual over time. Thirdly, the use of polar, water-soluble/hydrophilic polyamines and similar hydrophilic donors can make such capsules too hydrolytically unstable, leading to premature degradation in aqueous media (e.g. as made and/or when formulated)—and so again are less storage stable as made in aqueous media, and/or at pH's away from neutral such as acidic pH3. Whilst, as noted above, this may be advantageous for biodegradation performance in some circumstances, this is a major disadvantage when longer term storage of capsules is as an aqueous dispersion or slurry (the form they are typically made in), or in formulated products (e.g. liquid detergents, fabric conditioners), and which are aqueous and may have pH extremes. Fourthly, if very high crosslink densities such as those with both acceptor and donor having high multifunctionality (e.g. tri-functional or higher for each reactant) were to be required for capsule shells, as is the case for some applications described in our invention herein where particularly robust capsules are needed, the classical interfacial polymerization route can be very limiting since as the two high functionality donor and acceptor molecules co-react and polymerize at the interface they will quickly form highly a crosslinked shell structure relatively early on in the reaction zone and this prevents migration or diffusion of further donor (e.g. amine or thiol) from the water phase into the polymerizing zone—so limiting donor (e.g. amine or thiol) and/or acceptor (e.g. acrylate) conversion and limiting the attainment of higher crosslinked structures for the shell, which are required for more robust (retentive until broken and/or stable on storage) capsules. This latter occurrence is one reason why it is usually required to use excess donor (e.g. amine or thiol) in such a classical interfacial polymerization, which as mentioned above has such significant drawbacks. Indeed, in the publication cited it is a likely reason why only a difunctional thiol was able to be used—and necessarily therein at 50% excess of a 1:1 equivalent group stoichiometry. This diffusion issue would have been a much greater an issue with a higher functionality thiol than a dithiol.

For the prior art relating to microcapsules from poly-β-amino esters or poly-β-thio-esters all such prior art where classical oil in water polymerizations are described for microcapsule formation follow classical interfacial polymerization processes which have limitations in achieving the higher crosslink density requirements for the most demanding of end product applications and need to use excess addition levels of one of the reactive reagents. None of the prior art in making capsules via such methods reports on multifunctional (higher than difunctional) for both donor and acceptor reactants. Furthermore, in such prior art, there is a need for one of the monomer reactants to be water soluble, which can be limiting in designing the final composition of the wall polymer structure especially for demanding home or personal care applications. Also none of the prior art on such amino- or thio-ester microcapsules claim or show a combination of such storage stabilities, with relevant biodegradation properties and with a successful encapsulation of a cargo with the attributes of imparting a noticeable bloom or release of cargo when triggered (e.g. when rubbed or application of pressure) after storage or after incorporation into a formulation. Furthermore none describe a combination of an amino- or thio-ester in the same microcapsule shell wall material and none describe an in-situ oil-in-water polymerization process (where all reactants are in the oil phase) for their production and its associated advantages for achieving the desirable balance of selected reactants and desired crosslink densities for the most demanding of applications.

The poly β amino-ester or poly-β-thio-ester or the hybrid poly-β amino-ester co-β-thio-ester, or the polyester/β-amino-ester or polyester/poly-β-thio-ester hybrid capsules of our invention show successful encapsulation and subsequent triggered release of fragrance with associated evidence for biodegradability or non-persistence over time in aquatic media according to OECD test methods and are made via a convenient in-situ process not requiring substantial excess amounts of reactants at low to moderate temperatures suited to volatile ingredient encapsulations in an oil-in-water process, and not requiring subsequent volatile solvent removal and not using undesirable isocyanate or acid chlorides nor requiring high temperatures at the encapsulation stage. Furthermore, they show a combination of fragrance encapsulation, biodegradability and storage stability in various formulated products or pH ranges.

Present invention relates to biodegradable microcapsules, particularly, microcapsules that: (a) can encapsulate and retain cargoes, which can subsequently be released by a trigger and/or released gradually, and particularly where such cargoes are, or contain, lipophilic or hydrophobic core materials such as fragrances, butters or essential oil or other oils or oil solubilized cargoes; and, (b) whose shell material(s) show evidence of biodegradation or non-persistence in the environment and in particular in environments that are aquatic based (waterways, rivers, surface waters, seawater, sludge, treated waters, etc.) and/or soil or compost based and (c) which are storage stable as made or in one or more end-product formulations.

Present invention further describes a route to make micron sized (and above) capsules (microcapsules) and can be used for encapsulating sensitive or plasticizing or volatile lipophilic or other hydrophobic ingredients or actives or such as oils, or fragrances or butters or oil solubilized ingredients. Said biodegradable microcapsule polymeric shell compositions can effectively be used in various applications including, but not limited to personal care products, home care products, etc.

Our approaches have surprisingly found that polymeric shell capsules can be made to encapsulate fragrances, oils etc. and other cargoes which exhibit lipophilic tendencies, compatibilities or behaviors and which are stable on storage in aqueous media such as ‘as-made’, or in aqueous formulations of various pH's and optionally containing surfactants or other additives, and yet which are able to biodegrade in common, ambient, water based environments after use. Insoluble materials can be encapsulated by dissolution or partial dissolution, or via dispersion or emulsification, in a lipophilic carrier or diluent additive.

Although polyesters are one polymer matrix that can be used it is not the only one and, not all polyesters or co-polyesters will be suitable. Those with certain degree of resistance to degradation, or which are so slow to degrade as to not be able to be considered as non-persistent are not suitable, since ultimately it cannot be predicted or expected that they will ultimately degrade in the environment. A common example of a polyester that would not be considered as biodegradable according to criteria herein described which could be considered as persistent in the environment if unmodified, is polyethylene terephthalate (PET)—known to be a more durable polyester and used in many applications as such. Other polyesters based on significant terephthalate or other high aromatic contents are also not biodegradable and this would be expected to be the case with those polyesters in the above prior art patents using polyesters made from terephthaloyl chloride. Typically, polyesters with high contents of terephthaloyl groups would not be biodegradable in aquatic or indeed other common end environments. The same applies to other polymers with significant contents of aromatic chains in the polymer backbone.

In addition, polyesters or other polymer types, which, after formation of the capsule shell are soft solids or viscous liquids, or which are readily plasticisable or solubilized in the liquid lipophilic cargo (after completion of the formation of the capsules), are also likely to be unsuitable for impractical for direct encapsulation of certain lipophilic or volatile cargos, though in some cases they may be suited to other cargoes which do not so plasticize or solubilize the shell walls. Also, very highly crosslinked polyesters linked by high contents of carbon-carbon crosslinks (bonds) or by non-biodegradable or non-hydrolysable links or chains (e.g. polystyrene chains as in the known crosslinked unsaturated polyesters), are also not biodegradable.

We have discovered that certain polyester or co-polyester structures can achieve a unique balance of being sufficiently hydrophobic or lipophilic to facilitate some compatibility with the lipophilic cargoes while enabling some biodegradation to either a certain level within a defined time and/or at a rate that would be indicative that ultimately biodegrade and not be persistent in the environment, including aquatic environments.

The key aspects of the process and its variants of one embodiment of the invention can be described as: A prepolymer, which is biodegradable in the chosen medium (such as seawater, river water, activated sludge, etc. or soil or compost) is synthesized, optionally, though not essentially, with particular reactive groups either in-chain or at chain end(s). Here the term prepolymer is used to describe any polymer or oligomer pre-made prior to the encapsulation process stage during which it is transformed to form a microcapsule shell, and which is biodegradable or hydrolysable and which is also initially compatible with the heated cargo (or cargo diluent mixture) as described below. The prepolymer will have a certain level or arrangement of hetero atoms such as O, or N, or S, or P in the backbone to facilitate hydrolysis or biodegradation at a later stage, typically after use. A prepolymer containing ester and/or β-amino-ester and/or β-thio-ester bonds, optionally with amide and/or ether and/or thioether and/or carbonate and/or urethane bonds, is preferred, though ensuring its structure and composition is necessarily biodegradable according to criteria herein described.

In another embodiment, the present invention provides a microcapsule shell comprising a branched or crosslinked polymer derived from an aliphatic polyester prepolymer selected from aliphatic polyester comprising at least one reactive unsaturation functionality present either at a chain end or distributed along the chain. The aliphatic polyester comprises a crystalline structure and is derived from at least one diacid, diester, diacyl chloride, or anhydride comprising C₂-C₂₀ aliphatic chain or branched C₂-C₂₀ aliphatic chain or combinations thereof and at least one diol comprising C₂-C₂₀ aliphatic chain or branched C₂-C₂₀ aliphatic chain or combinations thereof.

Accordingly, the aliphatic polyester is derived from at least one diacid or multifunctional acid, preferably selected from the group consisting of succinic acid, malic propanedioic acid, butanedioic acid, hexanedioic acid, octanedioic acid, decanedioic acid, sebacic acid, dodecanedioic acid, octenyl succinic acid, itaconic acid, maleic acid and dodecenylsuccinic acid, or at least one anhydride selected from the group consisting of succinic anhydride, dodecenylsuccinic anhydride and octenyl succinic anhydride; and at least one diol selected from the group consisting of ethylene glycol, propylene glycol, butanediol, pentanediol, hexanediol, octane diol, decanediol, cyclohexane dimethanol, isosorbide, neopentyl glycol, ethyl hexane diol and dodecanediol.

The prepolymer is melted or dissolved (with warming if needed) into the cargo (optionally with added diluent or carrier) and so is also necessarily designed to be compatible with the cargo or a mixture of the cargo and a diluent, when heated. Optionally co-reactive reagents (that may react with the particular reactive groups, in-chain or at chain ends and/or aid solubilization) or crosslinking initiators (free radical for example) and/or other catalysts or accelerators may also be incorporated and/or additives that may aid crystallization for example, or that enable formation of complexes, salts or other forms of interactions with the prepolymer to transform it during the capsule shell formation process. An inert biodegradable polymer additive may also be incorporated as an option, so making a polymer shell wall with a blended mixture of polymers.

The prepolymer may contain reactive groups, such as unsaturated groups, at chain ends or distributed along the chain, which can be used for the transformation of the prepolymer during shell wall formation. Accordingly, the reactive unsaturation functionality is selected from the group consisting of acrylate, methacrylate, itaconate, citraconate, maleate, fumarate, crotonate and combinations thereof.

The polymer-cargo mixture (oil phase, with optional diluent) is mixed with an aqueous phase which can be solely water or water with added stabilizers or other additives. Optionally co-reactive reagents (that may react with the particular reactive groups, in-chain or at chain ends) or crosslinking initiators (free radical) are also incorporated and/or additives that may aid crystallization for example or that enable formation of complexes, salts or other forms of interactions with the prepolymer. The mixture is homogenized or stirred vigorously to form an emulsion while warm or heated.

The capsules may be formed during the stirring or homogenization and/or on cooling down to ambient temperatures or below. An insoluble polymer (insoluble in the cargo and insoluble in water) shell wall is formed for example either via crystallization or solidification or precipitation of the polymer or prepolymer on cooling and/or via crosslinking or chain extension or branching reactions between prepolymer molecules and/or between prepolymers and added co-reactive reagents, and/or via molecular rearrangements, and/or via complexation or formation of ionic salt bonds or interactions that link prepolymer molecules together and/or prepolymer molecules to added reagents.

Variation in orders of addition are also encompassed. For example, the prepolymer with stabilizer (polymer such as polyvinylpyrrolidone, PVP) and/or particle (such as a silica) are added and then a water—polyvinyl alcohol (Poval)—fragrance mixture is added and the whole homogenized. This can be undertaken at ambient temperatures or with heating.

Mixtures of stabilizers may be used (and incorporated at various points) and mixtures of stabilizers with added polymers to complement them and/or aid control viscosity or stability. For example, stabilizers used alone or as part of a mixture, may include polyvinyl alcohols, polyvinylpyrrolidones, hydroxethyl celluloses, hydroxypropyl celluloses and other cellulosic derivatives, guar, guar derivatives including cationic guars, gums including xanthan gum and the like, starches and starch derivatives, and/or any known emulsifier or dispersing aid and including particles such as silicas. Particle stabilized (Pickering emulsion) approaches are also able to be used. Defoamers are also used which may include liquid hydrocarbons, oils, hydrophobic silicas, fatty acids, alkoxylated compounds, polyethers, polyalklylene glycols, and nonionic emulsifiers.

This process outline and its incorporated variations can be applied to produce a microcapsule which has a shell wall which is biodegradable in the chosen medium and yet can encapsulate and retain a lipophilic cargo.

The microcapsule according to the above descriptions is formed wherein a polymer shell is formed around the cargo by a process of solubilization of a biodegradable pre-polymer or pre-oligomer, containing ester and/or β-amino-ester and/or β-thio-ester bonds, optionally with amide and/or ether and/or thioether and/or carbonate and/or urethane bonds, in a lipophilic cargo, optionally with added diluent or other reagents and/or heating, then emulsifying with water, and effecting a transformation of the pre-polymer or pre-oligomer such that it becomes insoluble in the lipophilic cargo, such a transformation being effected either via reactions, molecular rearrangements or interactions, and/or phase or solubility transitions of the pre-polymer or pre-oligomer within or from its mixture with the fragrance and diluent, if present and yet wherein such transformed polymer (capsule shell wall) encapsulates the cargo and still remains biodegradable or non-persistent in the environment. The diluent or solvent is selected from the group consisting of hydrocarbon oil, alkanes, an ester oils, a fatty acid esters, an aliphatic esters, and alkylene carbonates. A microcapsule with a biodegradable shell wall is thus produced according to the above wherein such a polymer shell is formed around the cargo.

In some cases, the products may be particles with entrained or absorbed or adsorbed cargo rather than fully formed capsules or may be capsules which function in both aspects. Entrained or absorbed cargoes are still retained though typically for shorter times compared to fully encapsulated cargoes in shell walls. A combination of entrained, absorbed or adsorbed cargo together with encapsulated cargo is also able to make in some cases. Also, the capsules or particles may form films on drying or casting or other processing which also contain and retain the cargo for certain times, all still being biodegradable.

In one embodiment, microcapsules that are formed in a slurry (typical initial reaction product mixture) with encapsulated cargo which are able to dry as capsules and then, if desired, be re-dispersed in water or aqueous media or formulations—and retained as capsules which are biodegradable.

In some embodiments, present application provides a prepolymer which is subsequently used in an in-situ oil-in-water microencapsulation process. Various reaction schemes for synthesis of biodegradable prepolymer or polymer compositions used are shown, including, but not limited to, Scheme-I to Scheme-V below:

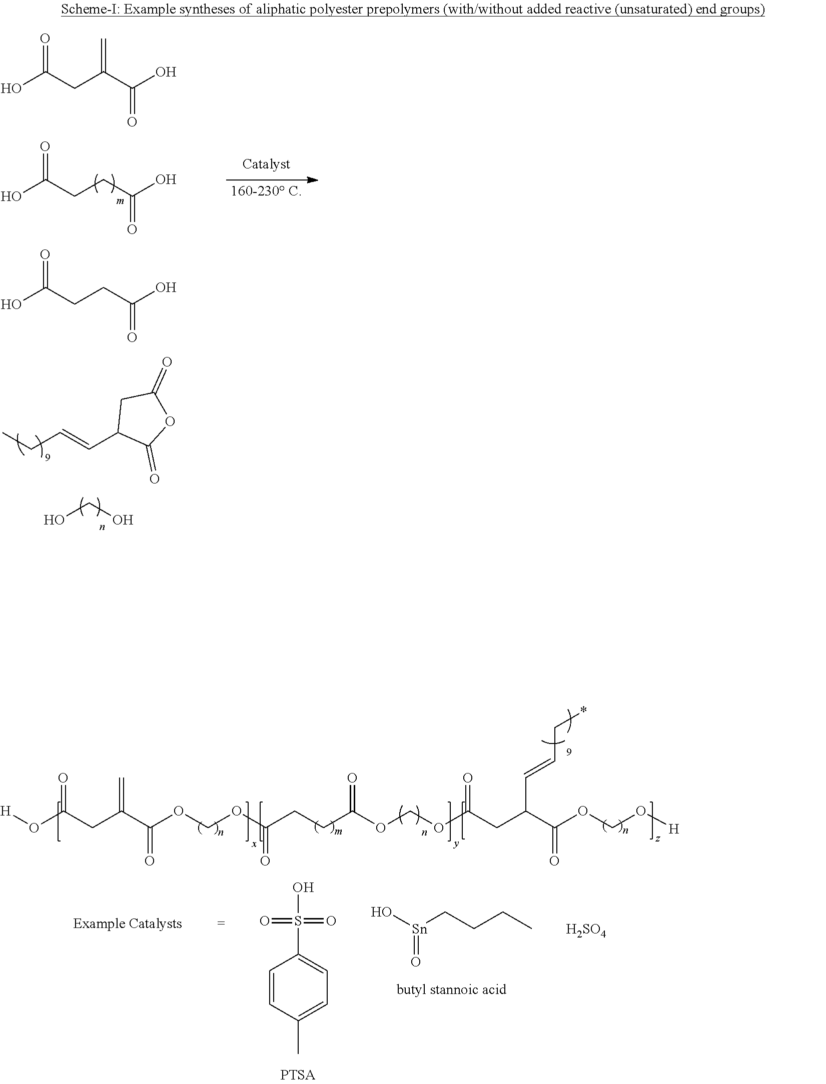

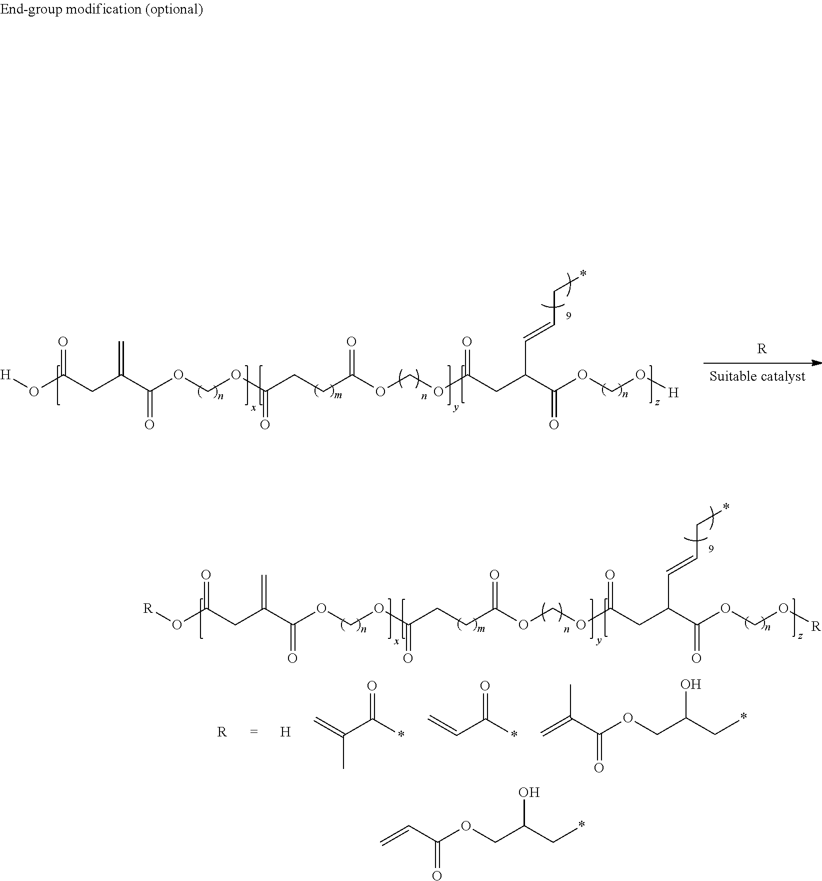

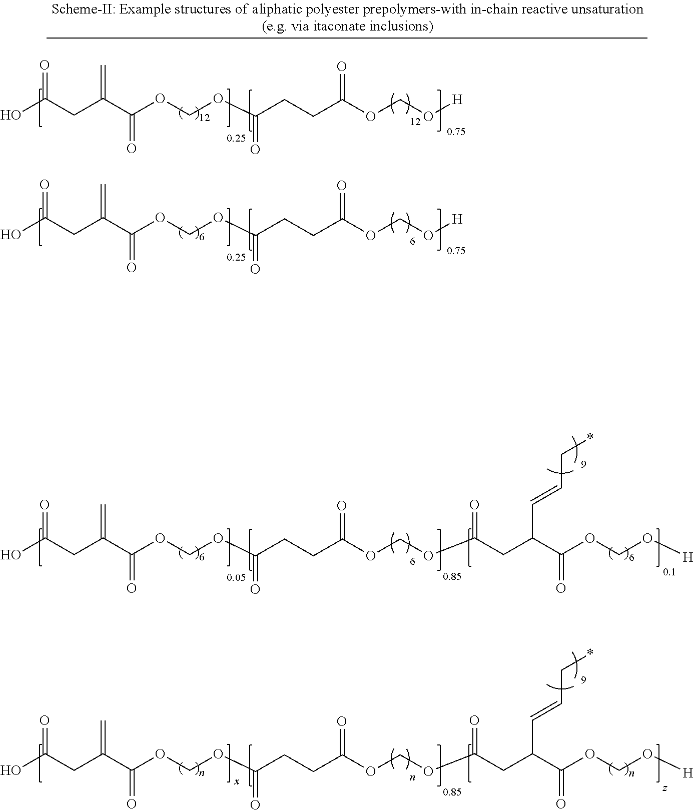

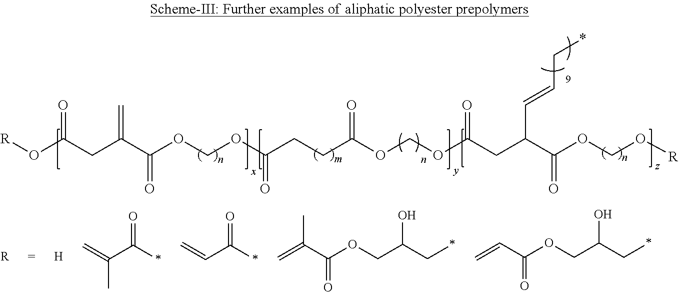

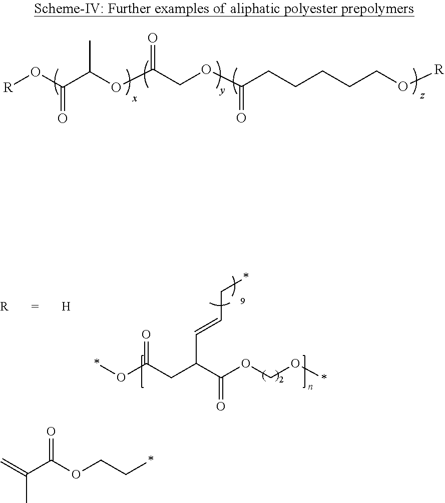

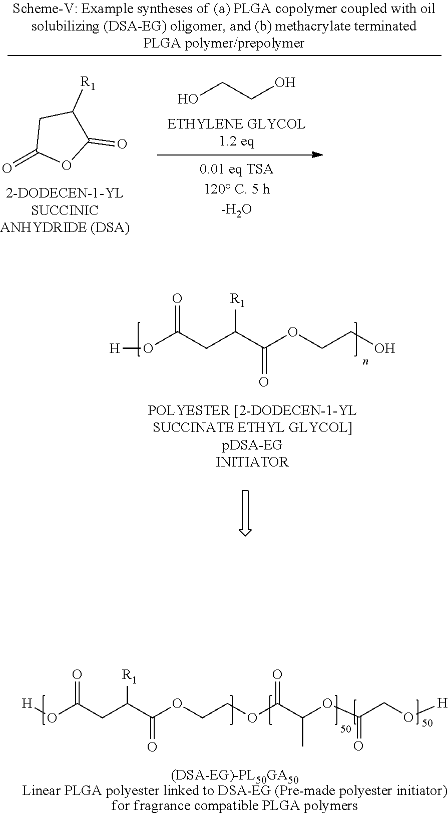

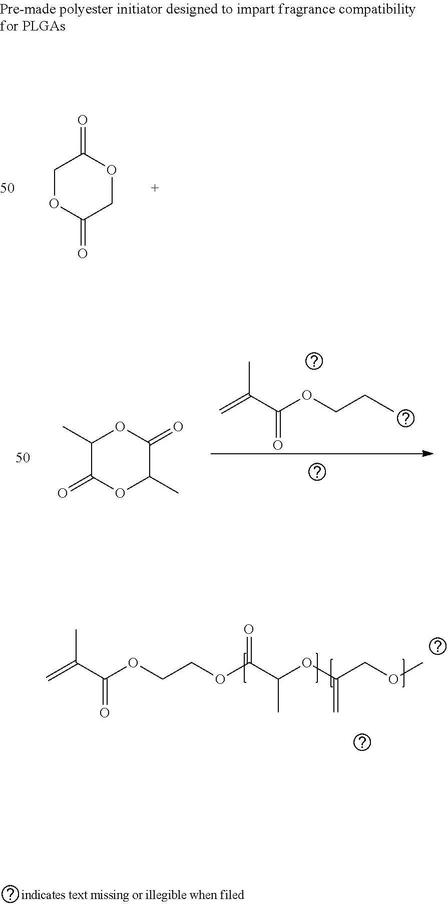

In other embodiments the polymer shells are built up from in-situ oil-in water-reactions of monomeric reactants. Schemes VI-IX below show some examples.

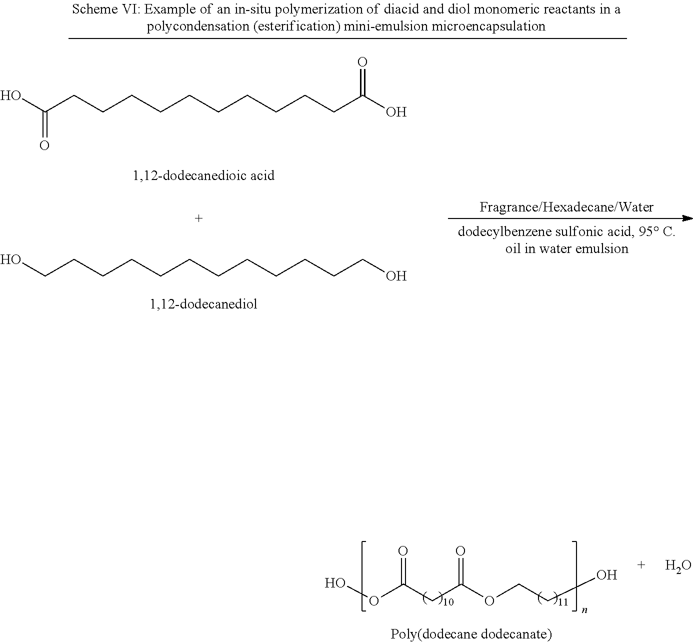

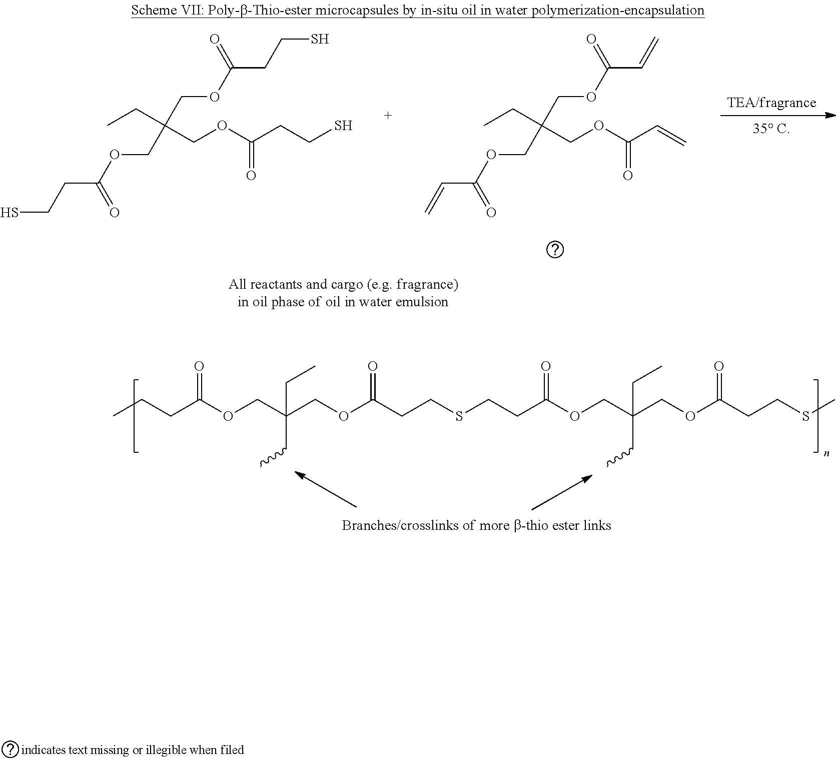

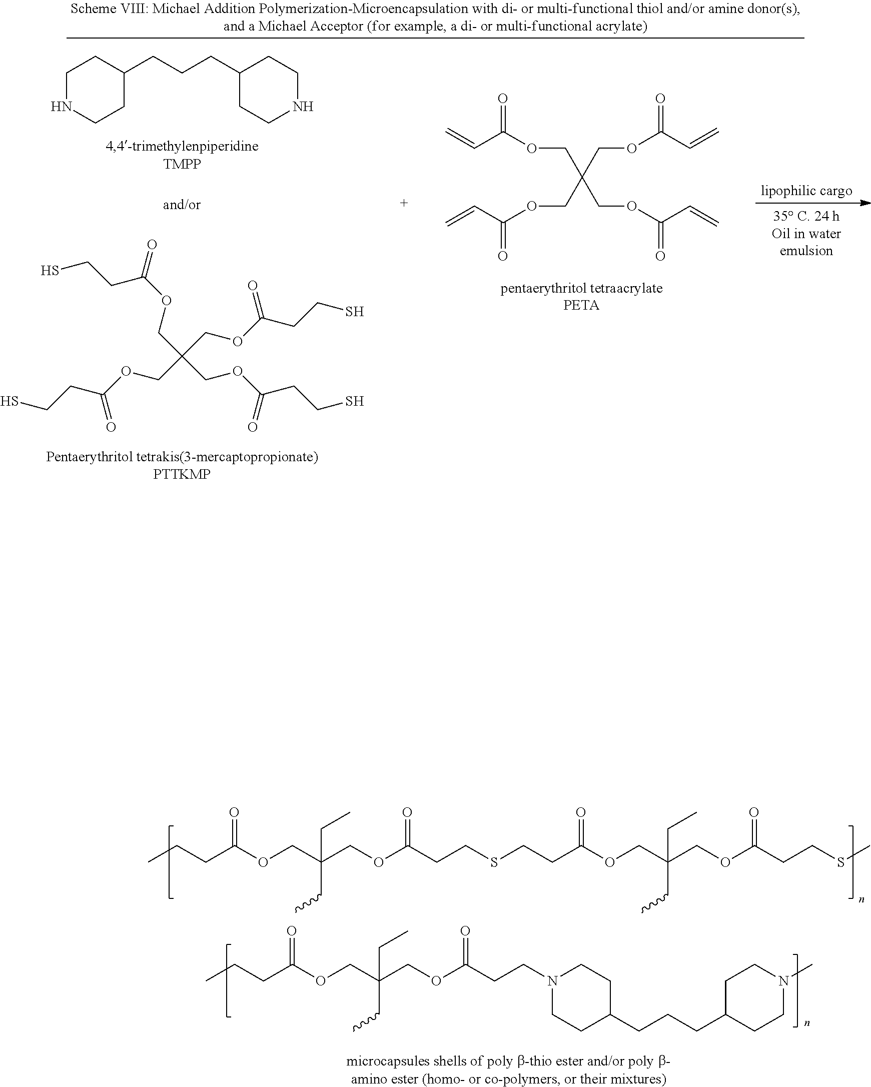

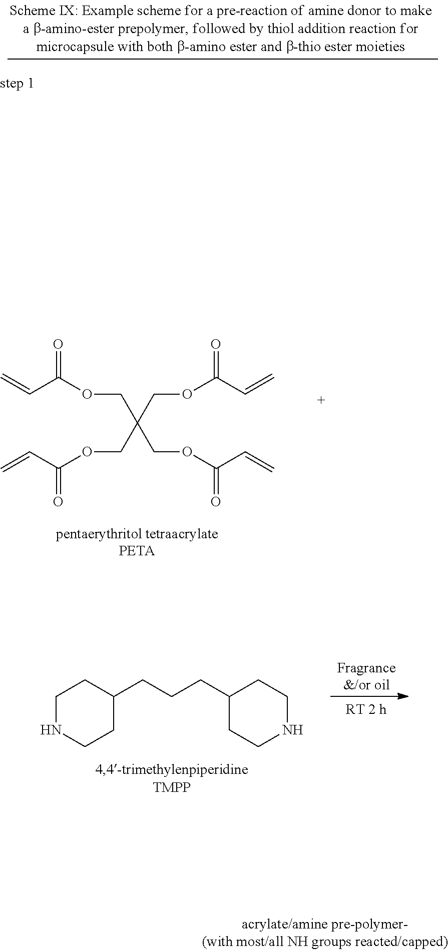 [SMILES: C=CC(=O)OCC(COC(=O)C=C)(COC(=O)C=C)COC(=O)C=C ; C(CC1CCNCC1)CC1CCNCC1]

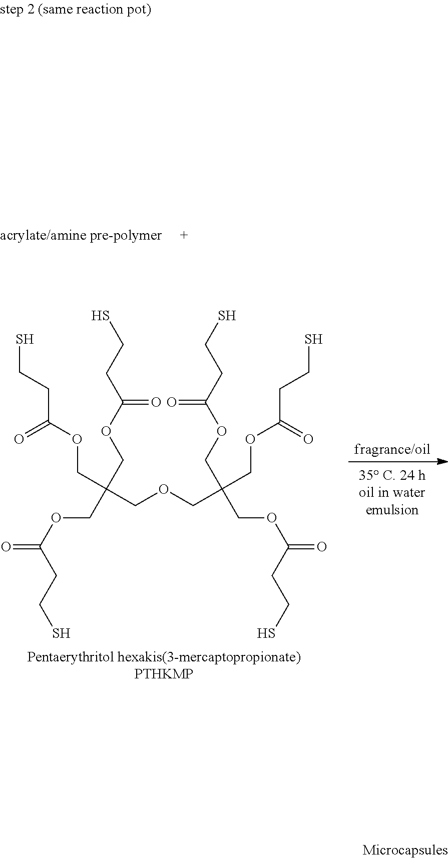 [SMILES: O=C(CCS)OCC(COCC(COC(=O)CCS)(COC(=O)CCS)COC(=O)CCS)(COC(=O)CCS)COC(=O)CCS]

Some prior art for encapsulating ingredients (typically non-volatile and/or thermally stable, typically pharmaceutical ingredients) using pre-made polymers, as starting materials for encapsulation, including some biodegradable polymers (usually biodegradable in bodily fluids/biomedical environments) typically use melt extrusion (which requires exposure to relatively high temperatures, so not suited to fragrances which will have volatile components) or use a solvent based process (which requires subsequent evaporation of the solvent, so not convenient for fragrances which will have volatile components)—when starting with polymers (larger molecules).

We have thus surprisingly found that certain polyester or co-polyester polymers which are pre-made, can be used in a subsequent (sequential one-pot or separate process) emulsion or dispersion encapsulation process, without the need to remove solvents or a need to heat to high temperatures (above 100° C.), and so can be transformed to form capsules via an in-situ oil-in-water emulsion process which can encapsulate volatile, hydrophobic or lipophilic or oil soluble ingredients and which furthermore which can be designed to biodegrade in aquatic or other environments over time according to criteria described herein, and concomitantly retain or contain or entrain fragrances or oils or related oil soluble or oil solubilized or other lipophilic cargoes.

In another embodiment, the present application provides a pre-made polylactide-co glycolide (PLGA) polymer, copolymer or terpolymer and the like, can be used in an oil-in-water emulsion or dispersion encapsulation process to make a biodegradable shell, according to criteria described herein, without the use of undesirable volatile solvents requiring subsequent removal and/or without the need for high temperature extrusion or related processes, by using an added benign (non-volatile but accepted for some end applications) diluent as carrier for the fragrance or other lipophilic or hydrophobic or oil soluble or oil solubilized cargo, and concomitantly retain or contain or such cargoes.

In one embodiment, the pre-made aliphatic polyester is a polymer derived from at least one lactide and at least one glycolide. The aliphatic polyester may be coupled with an attached oil solubilizing oligo ester or polyester chain. The oil solubilizing oligo ester or polyester chain is polyester comprising an alkyl side chain of C₂-C₂₀ aliphatic chain or branched C₂-C₂₀ aliphatic chain or combinations thereof or is an oligo- or poly-caprolactone.

In another embodiment, the aliphatic polyester is a polymer derived by ring opening polymerization of a lactide or a glycolide or a combination of the two, coupled with an attached oil solubilizing or solvent solubilizing oligo ester or polyester chain used as a co-initiator or linked through copolymerization or a reactive coupling.

Retention times of a fragrance or other cargo in such a capsule after application to a fabric or surface can be varied—shorter times are typically attained with linear polymers (that is typically without crosslinking after end-capping), though with some exceptions, where a more robust shell is formed after the encapsulation stage either via the inherent chain rigidity/crystallinity or via chain extension to build molecular weight in the encapsulation stage in some examples so resulting in a longer lasting fragrance effect after application. With crosslinking a more robust shell is typically formed and retention is more long-lasting. Similar affects are also observable with ionic salt based interactions within or between capsule shell components, and/or where crystalline domains are formed on cooling capsules once formed. Biodegradation times are typically longer in such cases, though over longer time periods evidence of non-persistence (for example in some circumstances showing ongoing biodegradation and attaining, after a time, a level of >20%) is able to be shown.

Examples of other cargoes that can be encapsulated through any of the embodiments, in addition to fragrances, perfumes, essential or natural oils and the like, including oil (ester or hydrocarbon) solubilized ingredients, liquids or low melting solids which are lipophilic esters, chlorinated solvents, hydrocarbons, insect repellants, and pigments, colorants, dyes, vitamins, antioxidants, lipophilic natural extracts or other actives which are oily or oil (ester or hydrocarbon) soluble.

Various other routes are also able to be applied to prepare capsules (microcapsules) of the invention, which can contain, retain or entrain a hydrophobic or lipophilic cargo, such as a fragrance or oil, and which can also be biodegradable in aquatic or other environments. As described above, the prior art also describes either use of small molecule monomers or precursors reacting in-situ, in an emulsion or dispersion process, to form a polymer or crosslinked polymer network in-situ from small molecules (monomers) such as acrylate monomers, or melamine-formaldehyde (M-F), or isocyanates with diols or diamines (for polyurethanes or polyureas) for encapsulating oils or fragrances with good retention. Here prepolymers are not necessarily made or required. We have discovered that biodegradable polymer shells for microcapsules encapsulating lipophilic cargoes such as fragrances oils and the like and which polymer shell walls comprise ester and/or β-amino-ester and/or β-thio-ester bonds, optionally with amide and/or ether and/or thioether and/or carbonate and/or urethane bonds present, can also be made through small molecule precursor (monomers) routes and not necessarily require a prepolymer to be made, though prepolymers may also be present in such approaches.

In such methods we have surprisingly found that certain polyester shells, or polyester compositions useful as microcapsule shells, that can be designed to be biodegradable according to criteria described herein, and which can contain, retain, or entrain fragrances or other lipophilic cargoes or oil solubilized cargoes, can be made by an in-situ polymerization-encapsulation emulsion (oil in water) polycondensation process starting from monomeric precursors such as diols and diacids, without the use of acid chlorides or isocyanates and/or without the need for long reaction times at higher temperatures, in the presence of fragrances and other lipophilic cargoes (oil phase), preferentially with all monomeric reactants in the oil phase from the outset.

In another embodiment, we have also discovered that, surprisingly, that certain highly branched or crosslinked polymeric shells that comprise β-amino-ester and/or β-thio-ester moieties and which contain, retain, or entrain fragrances or other lipophilic cargoes or oil solubilized cargoes, can be designed to be biodegradable according to criteria described herein, and can also be made by an in-situ polymerization-encapsulation emulsion (oil in water) process, preferentially with all monomeric reactants in the oil phase from the outset, without the need for water soluble precursors and/or without the need for large excesses of reactive monomers and/or without the need for long reaction times at higher temperatures.

In some embodiments, the polymer or crosslinked polymer is a poly-β-amino-ester or a poly-β-thio-ester or any combination thereof, derived from a Michael or conjugate addition reaction of a donor and acceptor, wherein the donor or acceptor has a reactive functionality of at least two or at least three.

In a non-limiting embodiment, polymeric microcapsule shell is derived from a donor-acceptor combination selected from the group containing: (i) a trifunctional, tetrafunctional, pentafunctional or hexafunctional thiol; and (ii) a trifunctional, tetrafunctional, pentafunctional or hexafunctional acrylate.

Another embodiment discloses that the crosslinked polymer is a poly-β-amino-ester or a poly-β-thio-ester or any combination thereof and is derived from a Michael or conjugate addition reaction of: (i) at least one multifunctional donor having a reactive functionality of at least three; and (ii) at least one multifunctional acceptor having a reactive functionality of at least three.

In one embodiment, the multifunctional donor and multifunctional acceptor each comprise at least one tri-functional, tetra-functional, penta-functional or hexa functional reactive groups.

In another embodiment, the donor is an amine or a thiol or mixture of the two. The donor is a mixture of at least one difunctional thiol or multifunctional thiol and at least one difunctional amine or multifunctional amine. The amine is a difunctional primary amine, a multifunctional primary amine, a difunctional secondary amine or a multifunctional secondary amine. The amine comprises a C₂-C₂₀ aliphatic chain, a C₄-C₇ cyclic ring or a C₄-C₇ heterocyclic ring.

According to another embodiment, the crosslinked polymer comprises poly β-amino ester, poly-β thio ester or copolymers thereof.

Non-limiting examples of the difunctional amine or multi-functional amine include 4,4′trimethylenepiperidine (TMPP), isophorone diamine, bis-(aminomethyl)cyclohexane, cyclohexane diamine, piperazine, aminoethylpiperazine, bis-amino-norbornane, diethylene triamine, diethylene diamine, tetraethylene pentaamine, hexamethylene diamine, diamino propane, diamino butane, decane diamine, dodecane diamine, and polyethyleneimine.

Another embodiment discloses that the donor is a mixture of one or more thiol and one or more amine, and the amine functional group (NH) is present in an amount of about ≤50 or ≤25 or ≤20% of total mole equivalent of thiol and amine functional groups (SH and NH).

In a different embodiment, the acceptor is selected from acrylate, methacrylate, maleate, fumarate, itaconate, malonate, crotonate, citraconate, maleimide or mixtures thereof. Preferably the acceptor is an acrylate. The acceptor can be an (i) acrylate, diacrylate, or multifunctional acrylate of an epoxide; an (ii) acrylate, diacrylate, or multifunctional acrylate of a urethane; or an (iii) acrylate, diacrylate, or multifunctional acrylate of a polyether; or combinations thereof.

Non-limiting examples of acceptor functionality is selected from the group consisting of trimethylol propane triacrylate, pentaerythritol triacrylate, pentaerythritol tetra acrylate, dipentaerythritol penta acrylate, dipentaerythritol hexa acrylate, or is an acrylate, diacrylate, or multifunctional acrylate of a polyester. The acceptor can also comprise difunctional acrylate.

Yet another embodiment discloses that the donor-acceptor combination further comprises difunctional amine, trifunctional amine, tetrafunctional amine, pentafunctional amine or hexafunctional amine. These may be secondary or primary amines.

One another embodiment discloses that the crosslinked polymer comprises combination of β-amino ester and β-thio ester, wherein the β-amino ester is present in an amount of about ≤50 or ≤25 or ≤20 mol equivalent % of total mole equivalent of thio-ester and amino-ester.

Thus, there are described below four non-limiting generic routes or embodiments for the practical application and implementation of the polymeric shell microcapsules of the invention which are able to encapsulate and retain lipophilic cargoes including aggressive cargo examples such as fragrances or volatile oils, while concomitantly such polymeric shells also being biodegradable according to the criteria described herein, and which are made via processes which, at the encapsulation stage avoid the need for high temperatures, and/or avoid the need to use or remove volatile or otherwise undesirable solvents or reagents. These four processes are:
(i) Prepolymer Route: Polyester or co-polyester or poly β-amino ester or poly β-thio ester prepolymer route with or without in-situ-crosslinking, branching or chain extension during the oil in water microencapsulation stage.
(ii) PLGA Prepolymer Route: Benign solvent mediated prepolymer route with PLGA polymers with/without in-situ crosslinking, branching or chain extension during the oil in water microencapsulation stage.
(iii) In-situ Emulsion Polycondensation Route: In-situ emulsion polycondensation of monomeric reactants or precursors (diols/diacids) route to form a polyester or co-polyester shell wall around the cargo. Accordingly, the in-situ emulsion polymerization includes polycondensation or esterification reaction of monomeric reactants to form a polymeric shell comprising an aliphatic polyester. The monomeric reactants are (a) at least one difunctional or multifunctional acid, acyl chloride, ester or an anhydride; and (b) at least one difunctional or multifunctional alcohol or a polyol. The in-situ polycondensation reaction of the monomeric reactants is carried out at a temperature at or ≤100° C. or ≤95° C. or ≤80° C. to form the aliphatic polyester polymeric shell. The catalyst is a sulfonic acid, phosphoric acid, or other acid, tin octanoate, tin hexanoate, stannic acid or a stannic acid derivative, tin oxide, or tin based compound or is a lipase or other enzyme.
(iv) Poly β-amino-ester and/or poly β-thio-ester route by in-situ oil-in-water addition polymerization: In-situ oil in water addition polymerization of monomeric reactants or precursors (donors/acceptors) to form a poly β-amino-ester and/or poly β-thio-ester shell around the cargo. The monomeric reactants comprise (i) at least one difunctional thiol, multifunctional thiol, difunctional amine or multifunctional amine donor and (ii) at least one difunctional or multifunctional Michael acceptor. For the more demanding of applications or cargoes, the in-situ reaction is between (i) a tri thiol, a tetra thiol, a penta thiol, or a hexa thiol; and (ii) a tri acrylate, a tetra acrylate, a penta acrylate, or a hexa acrylate. Accordingly, the method can additionally employ a radical initiator system added to water phase, oil phase or both phases at the start or part way through or near completion of the in-situ reaction. In this method, polymer is added as powder or solution to either water or oil phase and wherein, the polymer is selected from a group consisting of an aliphatic polyester, chitosan, cellulose, cellulose based compound and a protein.

Thus, in the range of process and/or compositional variations, embodiments, descriptions and examples of the microcapsules of the invention it will be understood that they are able to be used for many types of lipophilic cargoes and in many media or applications (formulated end products, including waterless or solid format products or solvent based products or formulations or in neutral or near neutral pH aqueous formulation media) and do perform in delivering some fragrances and/or other cargoes more readily encapsulated or retained and/or stored, while also showing biodegradability or non-persistence. However, in some cases such biodegradable microcapsules may not meet the most demanding of requirements, while retaining their biodegradability, for some fragrances or strongly solvating or plasticising cargoes retention in formulated liquid product media at pH's significantly away from neutral such as pH3 and/or in the presence of some aggressive surfactants or other ingredients, as might be experienced, for example, in liquid fabric conditioners, or in some solvent based formulated end products. Many of the uncrosslinked biodegradable polymeric shell examples may not meet the most demanding combinations of requirements, Indeed some of the more lightly crosslinked, branched or chain extended polyesters which do show evidence of biodegradability or non-persistence and which do make capsules that show some stability in some end formulations and that show performance on release of some cargoes—also may not do so in the most demanding combinations of media for storage or delivery (aggressive from outside) and/or with a strongly plasticising fragrance or cargo inside (aggressive from inside). In such more demanding circumstances, a high crosslink density capsule shell is likely required to retain a cargo, stably, on storage in such situations.

In another embodiment, the lipophilic core is selected from the group comprising agrochemicals, aliphatic esters, anti-microbial agents, anti-fungal, anti-fouling agents, antioxidants, anti-viral agents, biocides, catalysts, cosmetic actives, dyes, colorants, detergents, edible oils, emollient oils, essential oils, fats, fatty acids, fatty acid esters, food additives, flavors, fragrances, hair care actives, halogenated compounds, hydrocarbons, insecticides, insect repellants, lipids, lipophilic scale inhibitors, mineral oil, oral care actives, organic solvents, organic esters, chlorinated solvents, pesticides, perfumes, preservatives, skin care actives, UV absorbers, vegetable oils and combinations thereof or where any active, lipophilic or not, is solubilized in or miscibilized with one or more of these lipophilic cores listed herein, to form the core. The lipophilic core or cargo is preferably fragrance, perfume or an essential oil.

When designing capsule shells for the most demanding of applications, for example encapsulation of fragrances for liquid fabric conditioner products, a higher crosslink density is typically required and/or some other form of rigidity and ‘solvent/chemical resistance’ or resistance to the more extreme pH's, in the shell polymer structure. This typically translates to an ability to achieve a noticeable fragrance boost (bloom) or release upon physical crushing or via other triggers, considered highly advantageous for such products. Furthermore, such capsules are of course also required to remain ‘intact’ as capsules with fragrance inside (note fragrance is an ‘aggressive solvating or plasticising cargo’ compared many others) and retained inside for relatively long time periods, until such a crushing or other triggered release in use (by consumers) occurs. More particularly they are also often required to be stable (‘intact’) when stored before ultimate consumer end-use in formulated products which might be of extreme pH's such as pH3 and/or long time periods and/or contain solvents or ingredients that might compromise the polymer shell wall. As described above on the prior art examples of capsule technologies reported to meet such demanding needs are melamine-formaldehydes (M-Fs) and crosslinked acrylates.

These form durable rigid capsule shells with long term storage stability in formulated aqueous media such as pH3 often also containing surfactants, as is the case for some liquid fabric conditioner products, and all in the presence of the ‘aggressive’ cargo (fragrance). However, as also described above in the prior art, these M-F or acrylate or related capsules are not biodegradable in aquatic media such as seawater, river/surface water or activated sludge, nor are they compostable according to recognized international standards (such as EN/ISO, ASTM, OECD etc). Furthermore, such highly crosslinked capsule shells are not readily made to be biodegradable while retaining performance (fragrance boosts) or storage stability. Our invention has discovered routes to make stable capsules (e.g. stable on storage until use)—so resisting the solvating or plasticising/softening effect from the inside (fragrance cargo) and resisting the effects of the formulation components which may be at an aggressive pH3 and/or contain a surfactant mix (from the ‘outside’ formulation medium), but which will also show biodegradation or evidence of non-persistence in water based media (aquatic systems) and still perform, for example as a fragrance booster (when fragrance is the cargo) when triggered.

In another embodiment, microcapsules are used in in home care (laundry products, cleaning products), personal care (hair, skin, oral products) and industrial sectors (such as coatings, adhesives, agricultural products, energy markets) and others. As such many different formulations or use environments are encountered.

In another embodiment, it is disclosed that the microcapsule is stable as a core shell capsule in an aqueous slurry, in a water-based formulation or in a solvent-based formulation. The microcapsule is storage stable as a core shell capsule in solid formulated or printed product. The water or solvent based formulation can be in the pH range of about 3 to 11, 3 to 6, 6 to 8, or 8 to 11.

In another embodiment, the microcapsules of the present invention is formulated into a laundry detergent, fabric softener, fabric conditioner, shampoo, hair conditioner, liquid soap, solid soap, skin deodorant, skin moisturizer, skin conditioner, hair or skin protectant, cleanser, sanitizer, cleaning fluid, dishwashing washing fluid or tablet, washing powder or tablet or liquid, and a cosmetic formulation.

In a specific embodiment, the microcapsule is used in a fabric conditioner composition or a laundry detergent composition.

The capsules of this invention which are biodegradable or non-persistent in aquatic tests and which show encapsulation are able to perform and be stable in many formulations, including water based formulations or solutions at various pH's and with various additives present including surfactants or salts, and also in solvent based formulations or products and also in dry or waterless or low water content products (tablets, larger capsules, powders or powder blends, gels). In making or formulating such products, the capsules of the invention can be directly incorporated as a slurry as is produced by the process of production or may be added as a dried product (e.g. the capsules may be spray dried or freeze dried or fluid bed dried or dried by any other drying process, to make dried capsules). Examples are given below of spray drying for example to make a free-flowing powder or to make an over-coated capsule.

For addressing the most demanding of requirements in terms of stability in some formulations, or in solvents, or in water based formulations away from neutral pH, one possible route, as described above, would be to make a higher crosslink density polymers but using polyesters (or other biodegradable polymer chains) as a major component of the polymer shell, and in particular polyester chains that are potentially biodegradable or non-persistent. However, it is well known by those skilled in the art that conventional crosslinking will typically slow down or inhibit biodegradation processes.

We have surprisingly found that some particular highly crosslinked structures (as capsule shells) which have achieved a higher cross link density can show a combination of being robust capsules (‘bloom’ performance and storage stability in aqueous formulations) can still be hydrolysable or biodegradable or show evidence of non-persistence over time when tested for biodegradability in aquatic media. The use of hydrolysable or cleavable crosslinks, in some cases, can surprisingly lead to achieving a combination of robust capsules, stable on storage in water-based formulations, yet which can show biodegradability in aquatic environments or test media.

Such materials showing evidence of biodegradability in aquatic environments will also be compostable.

For fragrance encapsulations via the in-situ polymerization route, it is not convenient to make crosslinked networks via reactions that need high temperatures (e.g. condensation reactions between acids and alcohols)—this is undesirable if encapsulating a volatile or reactive cargo such as a fragrance, in-situ. Michael addition reactions are another known route to achieve crosslinking at moderate temperatures. Typically, an amine (primary or secondary) or a thiol donor reacts, under mild conditions, with an acceptor, typically a molecule with a conjugated double bond such as an acrylate, methacrylate, itaconate, maleate, fumarate or maleimide, among others. Polyamino-esters (Aza Michael reaction) or poly thio-esters (Thio-Michael reaction) are typically formed.

However, we have further discovered that a polymer shell or a hybrid or copolymeric polymer shell structure which contains both amino-ester and thio-ester moieties, in a crosslinked network, can be designed to have enhanced stability in pH's away from neutral compared to an analogous polyamino-ester polymer per se, and through selection of the suitable multifunctional combinations robust capsules can be made for encapsulating fragrance which are also, at the same time, storage stable, and also able to be biodegradable or show evidence of non-persistence in aquatic or other environments or media.

Furthermore, we have discovered that such an approach can surprisingly achieve high crosslink densities in making capsules [which are required for a boost or bloom performance in some applications, and also for storage stability in aqueous formulations that might be considered aggressive (destabilizing) in their pH and/or surfactant use] and yet still be biodegradable or non-persistent in aquatic media after use, as evidenced by OECD or other tests for example. Such stable (on storage) crosslinked capsule structures for the most demanding of encapsulations are able to be formed by the use of tri- or multi-functional reactants in the in-situ Michael reaction—and these so formed capsule shells are still biodegradable or non-persistent in aquatic media.

In terms of achieving sufficiently crosslinked materials for some applications, for a combination of storage stability in relatively aggressive (e.g. away for neutral pH optionally with surfactants and/or salts present) water based media, and a fragrance bloom in terms of performance as well as maintaining biodegradability after use or non-persistence over longer time periods, the required crosslinking can be achieved by for example using two or more functional reactants with a functionality of 3 or more, so designated as A₃+B₃ or A₃+B₄ etc. Other (lower functionality) reactants may also be present. For applications requiring the more robust shells preferably at least one of the multifunctional reactants will have reactive functionality of three or four or more. More preferably both of the multifunctional reactants will have a reactive functionality of three or four or more—in order to achieve capsules which can produce a fragrance bloom, and which are stable in fabric conditioner and similar low pH media.

We have thus discovered that these drawbacks associated with some uncrosslinked or lower crosslink density capsule shells such as sensitivity to, or storage sensitivity to, a more strongly plasticizing cargo and/or to an aggressive formulated end product, while still retaining biodegradability or potential for non-persistence according to criteria described herein, can be overcome by the alternative approach described—in-situ Michael Addition oil in water polymerization using high functionality reactants [for example with A₃ and B₃ monomers, i.e. trifunctional or more in each of at least two of the reactants. Herein described as one embodiment of our invention is an in-situ oil in water encapsulation via a Michael addition polymerisation in the oil phase with a multifunctional donor and multifunctional acceptor, with other (lower e.g. di) functionalities optionally also present. Such an approach has the major advantage of allowing for the selection of more lipophilic donor and acceptor monomers and including monomers (acceptors and donors] with higher functionalities, which in turn reduces the risk of excess residual monomer and ensures that high crosslink densities and suitably retentive and stable capsules are obtained or can be more readily tailored. Furthermore, early gelation is limited due to dilution (solvation) in the cargo (oils, fragrances or other hydrophobic cargoes).

In another embodiment, the present polymeric microcapsule shell is biodegradable in an aquatic medium or solid medium or is compostable. The aquatic or solid medium is selected from group consisting of activated sludge, secondary effluent, river water, surface water, fresh water, sea water, soil and compost.

Another embodiment of the present application discloses that the inventive polymeric microcapsule shell material shows a biodegradation rate of at least 20% in an aquatic medium when measured by an OECD Test method 301, 302 or 306. The polymeric microcapsule shell material shows evidence of biodegradation within 120 days or within 60 days or within 40 days or within 28 days.

For the most demanding aqueous media for storage or delivery of the capsules such as pH 2 or 3 or pH 11 or 12 higher crosslink densities are preferred and yet surprisingly the microcapsules made can be biodegradable or non-persistent according to OECD or other standard tests. Preferably more than 50 mole % of the composition will be trifunctional or more, more preferably more than 60 mole %, and more preferably more than 70, 80 or 90 mole %.

For many monomer combinations, an in-situ oil in water polymerization can lead to a core-shell morphology, and if sufficiently highly crosslinked (higher functionality donors and acceptors), lead to robust capsules for good fragrance retention, and/or good long-term stability and/or bloom (burst) of fragrance release following deposition on to fabric, including cotton swatches for tests, and breakage through friction, for example.

For the in-situ polymerization process thiols and/or amines (and other donors, and also acceptors) which are of a lower water solubility, compared to for example common multifunctional aliphatic amines or the more highly water soluble thiols, are among the donors that are preferentially used in some embodiments. They tend not to partition into the water phase during the reasonably rapid polymerization (reaction) stage. More choice exists in polyfunctional thiol candidates for this but some polyfunctional amines are also suitable where they have relatively lower water solubility or hydrophilicity and are more significantly present in the oil phase (where the cargo and other reactant are also present). Tetramethylpiperidine (TMPP) is one example of lower water soluble multifunctional amine but any others which do not substantially partition into the water phase can be used.

One potential drawback of in-situ polymerizations compared to the interfacial route is the limitations on the use of relatively highly water-soluble reactants (monomers). While one such monomer is required (for one of the monomers) for interfacial polymerizations, when a highly water soluble monomer (donor or acceptor) is used in an in-situ oil in water polymerization, where all reactants are in the oil phase, there is potential for it to partition out into the water phase and so not fully participate in the in-situ polymerization (which takes place in the oil phase). In some cases that may limit the network structure and limit formation of a more robust shell. This can lead to non-formation of core shell capsules or of soft capsules, or a partial collapse of the capsule structures or it may lead to a more open (loose) structure or a matrix type of capsule structure, typically less well performing in terms of robust cargo retention or of storage stability.

We have discovered a route to overcome this in some systems where in-situ oil in water polymerization is used, and where a more water soluble (partially partitioning) donor (or acceptor) molecule is desirable to use, for example to control a biodegradation profile, and so incorporate it into the capsule shell structure. Thus, in another embodiment any amine, including hydrophilic amines, can be incorporated into the capsule shell polymer by an approach whereby the amine is pre-reacted, in bulk or with an oil carrier present, via a Michael Addition with a polyfunctional acceptor (e.g. multifunctional acrylate or itaconate) such that the NH bonds are largely or completely capped via the pre-reaction with the acceptor molecules. This so forms, essentially, a new acceptor in-situ which contains the amine derived moiety within it and which can be further reacted in a subsequent Michael Addition reaction (the in-situ polymerization stage, where cargo is present along with the remaining reactive monomer(s)). This can all be done sequentially in the same reaction vessel if desired. The in-situ polymerization stage is then with another donor which is typically a less hydrophilic option such as a polyfunctional thiol, for example, and wherein preferentially the overall stoichiometry is largely matched so that the double bond acceptor groups now hanging off the amine from the first reaction step will be reacted with all of the added new donor groups e.g. thiols, added at a ratio that largely maintains an overall stoichiometry of about 1:1 of donor to acceptor molecules in the entire structure.

In this way, by doing such a pre-reaction, a hybrid Michael Addition polymer of a poly-amino ester co-thio ester (or copolymer of β-amino- and β-thio-esters) is able to be produced via in-situ oil in water polymerization with all reactants in the oil phase, with any amine, even water soluble ones, and this can be beneficial to tune biodegradability performance and capsule performance in terms of fragrance release bloom testing and fragrance storage stability even in pH's away from neutral. A pre reaction of a water-soluble amine can be incorporated as a first step [separate or integrated (one-pot)] in the process as a precursor step in the overall process. In some cases a similar effect can also be achieved, for amine donors, by making an oligomeric amide with amine end groups (made via excess amine functionality in a reaction with a difunctional or multifunctional acid or acid derivative)—so making a precursor adduct wherein the amine is slightly chain extended via amide formations or other reactions to make a less water soluble molecule containing the amine moiety but in this case, with amide bonds present and amine (NH) and groups retained (for subsequent reactions in the subsequent in-situ polymerization phase).

Without being bound by theory it is suggested that a more biodegradable shell made from β-thio esters, can be produced via incorporation of a polyamine donor (forming β-amino ester moieties in hybrid or copolymer structure with β-thio esters) and/or via selecting a more hydrophilic or labile polyfunctional amine for that step.

Similarly, a lower overall functionality (as determined by the overall total multifunctionality of the system), from all donors and all acceptors in the polymerization, resulting in relatively lower crosslink densities would likely result in a more biodegradable shell other things being equal.

In most cases overall stoichiometry is preferably largely maintained at or around 1:1 in terms of overall donor and overall acceptor reactive (functional) group equivalents. Variations can be accommodated, small or large, though smaller variations are preferred and for example within about 20 or 10 mol equivalent % or within 5 mol equivalent % or within 1 mol equivalent %.

In the use of the Michael addition reaction approach, a mixture of thiol(s) and amine(s) as donors can be used advantageously to tailor a balance between biodegradability and encapsulation performance or stability on storage, including in pH's at or away from neutral such as 3 or 11, and including in formulated products such as liquid fabric conditioners/softeners, shampoos, soaps, deodorants, skin creams, insect repellent delivery, cleaning fluids, sanitizers, agricultural active delivery, among others.

In another embodiment, the present application provides a method for preparing microcapsules comprising polymeric microcapsule shell prepared from β-thio ester and β-amino ester functionalities, the method comprising: a) pre-reacting a difunctional or multifunctional amine with difunctional or multifunctional acrylate, b) preparing an oil-in-water emulsion of (i) an oil phase comprising the product of (a) and any remaining acceptor mixed with a difunctional or multi-functional thiol, and at least one lipophilic core, optionally with a diluent; and (ii) a water phase comprising at least one stabilizer or emulsifier, optionally adding at least one catalyst to the oil phase or water phase, c) forming the polymeric microcapsule shell wall by an in-situ oil-in-water Michael addition polymerization reaction of the donor and acceptor reactants, and d) obtaining the core encapsulated in a polymeric microcapsule shell.

It will also be understood that the capsules of the invention, and through any of the embodiments or variations, can be dried or made into coated or double layered capsules. This can enhance storage stability further and/or performance further. The double layered, multilayered or over coated microcapsule comprises a hydrogel or a crosslinked alginate. Examples of such concepts are described further below. Microcapsules of the present application have an average diameter of about 100 nm to 100 μm though distributions can span outside of this range and capsules can be made larger if desired. More typically average particle size ranges from about 1 μm to 100 μm. By varying reaction conditions and relative concentrations, particle sizes can be varied. All examples below fall within these ranges.

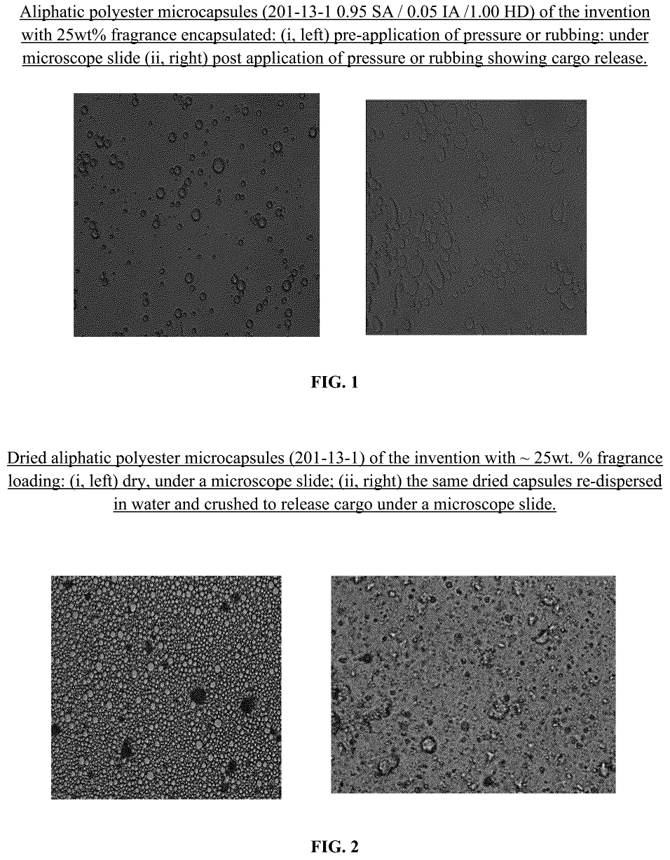

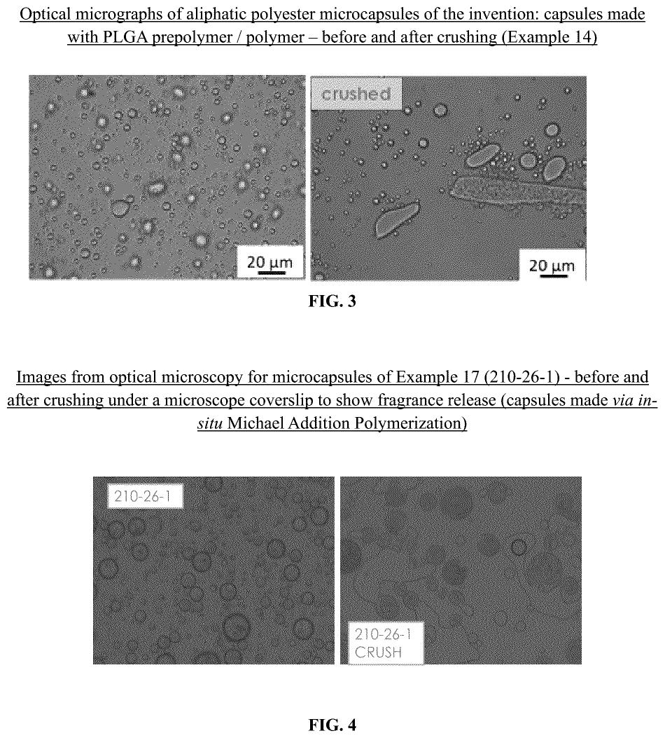

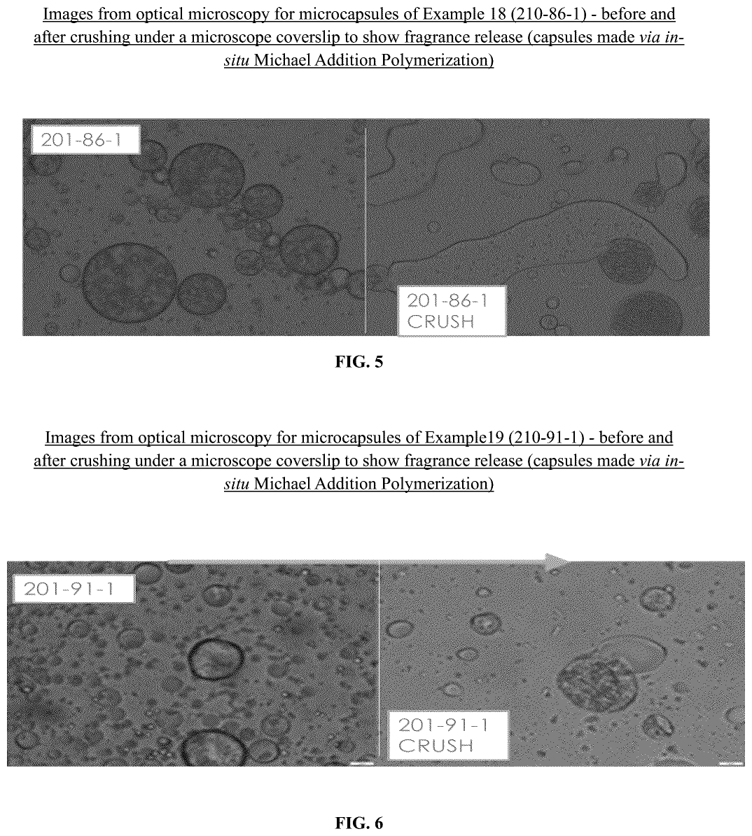

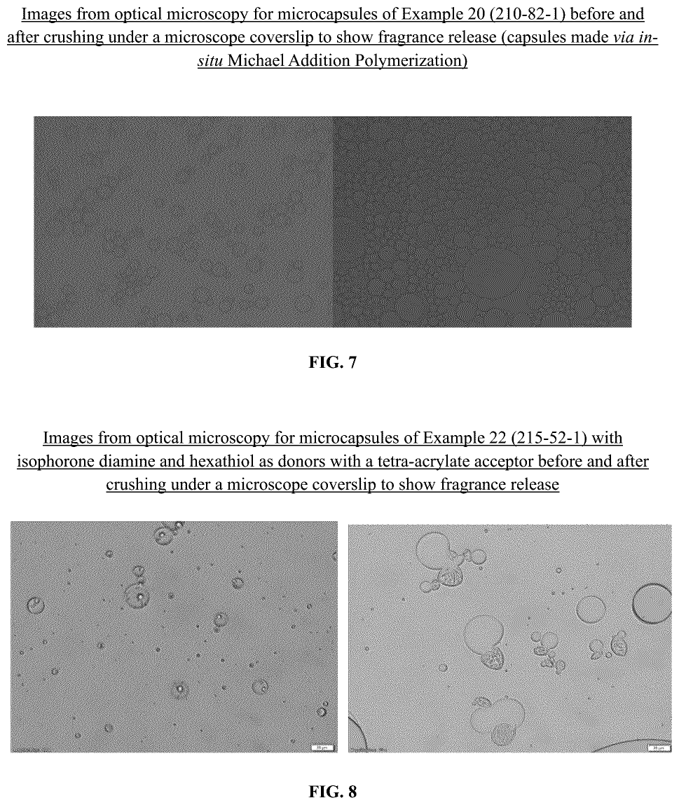

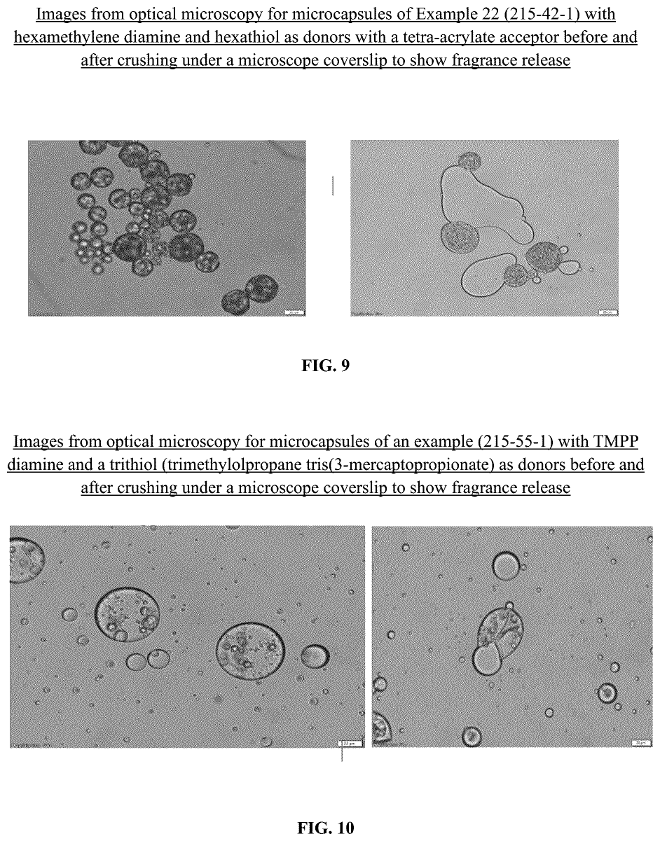

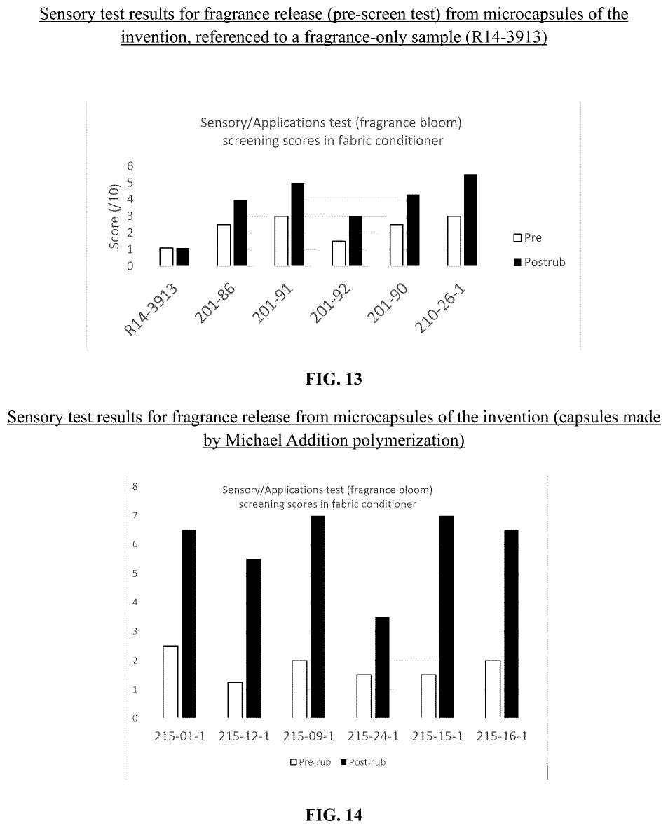

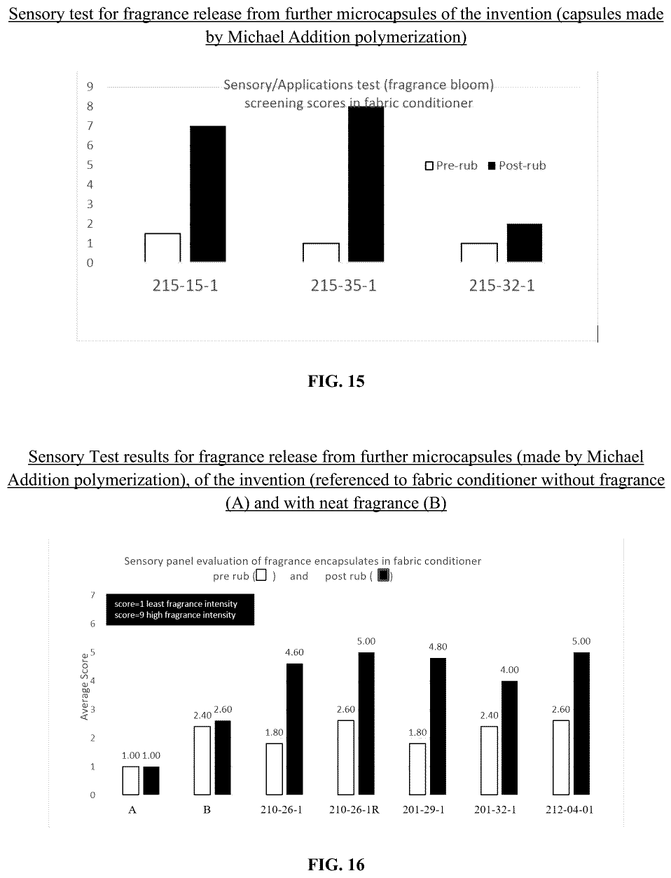

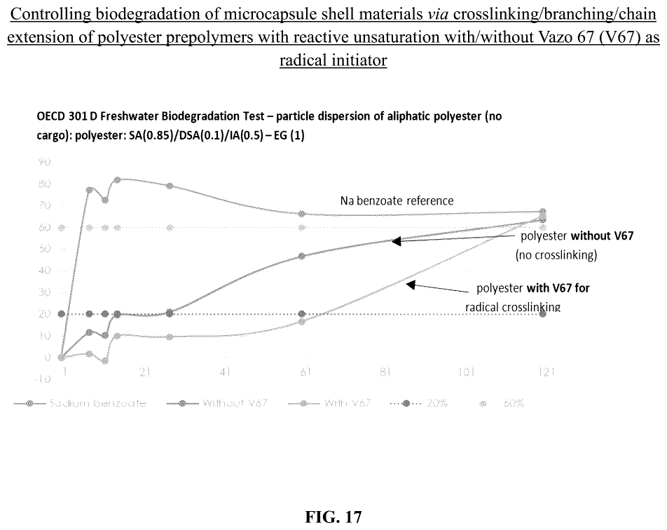

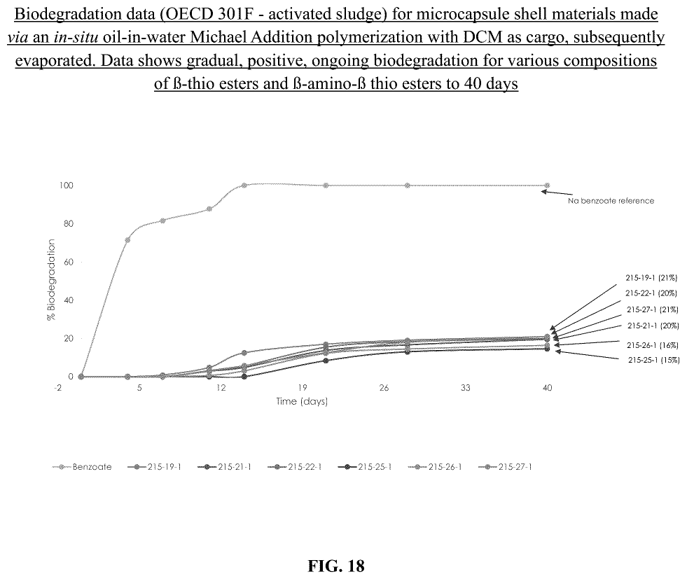

Further, certain aspects of the present application are illustrated in detail by way of the following examples. The examples are given herein for illustration of the application and are not intended to be limiting thereof FIGS. 1-12 show optical microscopy images of examples of microcapsules made using various polymers and via various processes described. FIGS. 13-16 show sensory test results for fragrance release from microcapsules prepared via the various processes described. FIGS. 17 and 18 show biodegradation data of microcapsule shell materials prepared by various processes described.

1. Prepolymer Route

In these routes, firstly, a prepolymer is synthesized or sourced which optionally has reactive functional groups either as end groups and/or distributed along the chain. This polymer is then used in a subsequent microencapsulation process, which can be a sequential process in the same vessel as the prepolymer synthesis or performed later in a new vessel. In these cases microencapsulation proceeds via initial dissolution of the prepolymer in the cargo, optionally with added diluent, (altogether forming an oil or organic phase), typically with warming, followed by emulsification of that oil phase with an aqueous phase, followed by either cooling or a reaction of the functional groups that may be present in the prepolymer either via self-reactions (e.g. free radical polymerization or crosslinking) or reactions with added co-reactive reagents which form chain extensions, and/or branches and/or crosslinks during the encapsulation. Any aliphatic polyester which is soluble or solubilized in the oil phase (with or without added diluent) of the encapsulation (oil in water emulsion) stage and which will be biodegradable once formed into the shell is able to be used though some such as those with reactive functionality for crosslinking or chain extension or with high (above 25 or 30° C. or higher) Tg or which have crystalline domains formed on cooling are preferred for the more robust capsules. Typical reactive functional group are vinylic (e.g. in acrylate/methacrylate, acrylamide methacrylamide, etc.) or epoxy-acid (e.g. acid or anhydride terminated prepolymer (e.g. difunctional in acid end groups) reacted with a diepoxide for in-situ linear chain extension, or a multifunctional epoxide for in-situ crosslinking via acid-epoxy reactions. Other reactive group combinations are also feasible, for example hydroxyl (diol)—isocyanate (di- or multifunctional) and all related variations. As another embodiment a reversible or non-covalent form of crosslinking or pseudo-chain extension can be performed via use of added di- or multi-valent bases such as the oxides of magnesium, calcium, aluminium, barium and the like which form complexes or ionic/salt interactions with acid end groups. A key aspect in such approaches which, during the encapsulation stage, build molecular weight or introduce intermolecular interactions or which add crosslinks or branching during encapsulation is that the chain-extended or linked or crosslinked prepolymer and/or its combinations with added reagents for in-situ chain extension (e.g. difunctional epoxide with polyester-diacid) or crosslinking (multifunctional epoxide with di- or multi-functional acid) during encapsulation, is that the polymeric shells remain biodegradable after reactions or interactions are completed that form the capsule shell.

In another embodiment, the method for preparing a microcapsule of claim 1, the method comprising: a) preparing an oil-in-water emulsion of (i) an oil phase comprising a polymer or a prepolymer, and at least one lipophilic core; and (ii) a water phase comprising at least one stabilizer or emulsifier, b) optionally adding at least one catalyst or at least one initiator to the oil phase, c) optionally heating the oil-in-water emulsion with stirring to a temperature between 25° C. and 100° C.; d) forming the polymeric microcapsule shell either by cooling or by an in-situ oil in water reaction of the polymer or prepolymer, and e) obtaining the core encapsulated in a polymeric microcapsule shell; wherein, the formed polymer or prepolymer is an aliphatic polyester or a poly-β-amino ester or a poly-β-thio ester or their co-polymers or ter-polymers or combinations thereof.

The prepolymer of the present application may contain unsaturated groups at a chain end or distributed along the chain and the in-situ reaction to form the polymeric shell includes reaction of the prepolymer containing unsaturated groups via (i) a chain extension, (ii) branching or (iii) crosslinking reaction.

The prepolymer contains conjugated unsaturated groups at a chain end or distributed along the chain and the in-situ reaction to form the polymeric shell includes Michael Addition reaction of the prepolymer containing conjugated unsaturation with a difunctional or multifunctional amine or a difunctional or multifunctional thiol via (i) a chain extension, (ii) branching or (iii) crosslinking.

The prepolymer contains reactive acid or anhydride groups at a chain end or distributed along the chain and the in-situ reaction includes reaction of at least one acid or anhydride group of the prepolymer with at least one difunctional or multifunctional epoxide or difunctional or multifunctional amine via (i) a chain extension, (ii) branching or (iii) crosslinking.

### Examples of the Invention Via the Prepolymer Route

Examples of the invention are described where microcapsules with a lipophilic core and a biodegradable polymeric shell are made by:—making an oil-in-water emulsion of an oil phase comprising an aliphatic polyester polymer or prepolymer, for example pre-made by a polycondensation reaction, and a lipophilic cargo, optionally with added diluent or solvent and/or aided by application of heat, optionally adding a catalyst or initiator to one phase, forming the capsule shell wall either by cooling or by an in-situ reaction of the prepolymer or polymer, and obtaining the cargo encapsulated microcapsule.

The diluent will preferably be a liquid at room temperature or readily meltable at moderate temperatures such as below 90° C. or less than 50° C. and may be a hydrocarbon oil, an alkane, a melted wax, an ester oil, a fatty acid ester, an aliphatic ester, or an alkylene carbonate. Some specific examples include mineral oil, long chain alkanes such as hexadecane and the like, aliphatic esters such as esters of long chain acids such as caprylates, myristates, oleates, cocoates, palmitates, or stearates including isopropyl myristate as one example, or long chain esters of shorter chain acids or other monohydric or polyhydric esters.

Where the prepolymer contains unsaturated reactive groups, the initiator is preferably a radical initiator which may be a peroxide or an azo based radical initiator or a redox system such as a persulfate based system or maybe a photoinitiator for UV induced radical reactions.

### Example 1: Synthesis of Polyester Prepolymers (Bulk Polycondensation)

Polycondensation polymerizations were typically conducted in bulk at temperatures between 130-230° C. under vacuum over several hours or days to achieve molecular weight build up, typically above 2,000 g mol⁻¹. Typical polyester syntheses procedures are described below for polyesters with all aliphatic backbones from diacids and diols. An overall 1:1 molar ratio of diacid to diol was used in some examples, though variations in this overall stoichiometry (diacid:diol) can be also be designed and accommodated to introduce acid- or hydroxyl-rich end groups through variations of the feed monomer stoichiometry (excess acid leading acid rich end groups; excess diol leading to hydroxyl rich end groups). Other end-capping procedures are also described below for such polymers, where required. Various times and/or temperatures also used in the examples. Diesters, or diacyl chlorides, or anhydrides may also be used in place of acids, though often acyl chlorides are preferably avoided for many applications they can be used in principle.

There are many diols or diacids, or acid derivatives which can be used to make polyester prepolymers and in combinations which combine an initial compatibility with the fragrance or oil cargo, features (self- or co-(with added reagents) reactivity, or other functionality, for chain extension, branching, crosslinking or other linking, and/or crystallisability that allow formation of a capsule shell with integrity to encapsulate such cargo and an ability to be biodegradable after capsule shell formation, especially in aquatic environments, (in some cases meeting OECD test criteria), This allows tailoring of cargo compatibility (required initially prior to capsule shell formation), and of stability of a dispersion or emulsion in water, and of (after capsule shell formation) biodegradability, and of retention of cargo for a period of time. If required, water dispersibility and/or biodegradability can be further improved by inclusion of citric acid, glycerol, oligomeric ethylene glycols, or other polyester precursors which have relatively greater hydrophilic properties.

Microcapsules (see further below—Example 5) were then (in the same pot sequentially or as separate step) prepared from such pre-made polyesters (polyester prepolymers) either directly, via an oil-in-water in-situ encapsulation process or after additional end-capping with reactive end- or in chain-groups then again directly via an oil in water in-situ encapsulation processor optionally with concomitant chain extension or branching or crosslinking during the in-situ oil-in-water encapsulation stage.

A polyester was synthesized with a mixed aliphatic backbone made using succinic acid (SA), dodecanedioic acid (DA), dodecenylsuccinic anhydride (DSA) and ethylene glycol (EG) as SA(0.5)-DA(0.25)-DSA(0.25)-EG(1) where the brackets indicate the molar ratios. Other variations are indicated below and/or in tables.

Succinic acid (SA; 0.5 eq, 48.3 mmol, 5.708 g), dodecanedioic acid (DA; 0.25 eq, 24.16 mmol 5.565 g), dodecenylsuccinic anhydride (DSA; 0.25 eq, 24.16 mmol 6.437 g), ethylene glycol (EG; 1 eq, 96.64 mmol, 6.000 g) and, as catalyst, para-toluene sulfonic acid (p-TSA; 0.01 eq, 0.96 mmol, 0.166 g) were added to a 250 mL round bottom flask and melted with stirring at 145° C. attached to a condenser and vacuum pump. Once melted, the vacuum was increased slowly over several hours to draw off the water produced by the step polycondensation polymerization (a minimum of 100 mbar). The reaction was continued for a few hours to 3 days depending on targeted molecular weight (MW). Analysis of the resultant polyester was via SEC in THF (tetrahydrofuran size exclusion chromatography) and by an acid number titration, which were carried out on small samples of the reaction product. For this example (˜24 hrs. reaction time; ref ENC 2188) the polyester prepolymer MW (THF SEC) data was: Mw of 5 200 g mol⁻¹, and Mn of 2 800 g mol⁻¹ and an Mw/Mn of 1.84.

Microcapsules (Example 5) were then prepared from this polyester prepolymer (SA (0.5)-DA (0.25)-DSA (0.25)-EG (1)) after end-capping with glycidyl methacrylate (GlyMA) to introduce a degree of cross-linking for the capsules. (See below). The synthesis of this polymer was repeated on a larger scale (˜100 g; ref ENC 2207) with similar product properties and was used in different end-capping approaches were used which each then were used to make capsules subsequently.

Tables or examples further below show a range of other prepolymers as examples [some subsequently end-capped (see below) and then used in microencapsulation]. Variations in reaction times and temperature and catalyst selection are shown. For example, FASTCAT 4100 (butyl stannoic acid) was used as a catalyst in a number of examples. Other catalysts can be used such as other tin or organometallic catalysts, sulfonic acids, or phosphoric acids.

### Example 2: End-Capping of Polycondensation Prepolymers

Selective end-capping of the polyester prepolymer with acid or hydroxyl groups can be designed by altering the reaction stoichiometry of diacid-diol in reactions above. Excess diacid will lead to diacid rich end caps in the prepolymer. Diacid end caps can then react, for example, with epoxy functional reagents, or other reagents with functional groups that will co-react with a carboxylic acid under moderate temperature conditions, for chain extension or crosslinking during the oil in water in-situ encapsulation stages. Diol end caps can react, for example, with isocyanate or anhydride functional reagents, or other reagents with functional groups that will co-react with a hydroxyl group under moderate temperature conditions, for chain extension or crosslinking during the oil in water in-situ encapsulation stages.

Diacid end cap groups can be introduced to prepolymer while maintaining, or slightly increasing, prepolymer MWs, by reaction of a 1:1 (diacid/diol) prepolymer with acid anhydrides (e.g. succinic, dodecenyl succinic, octenyl succinic, maleic anhydrides etc.) at the end of the initial (1:1) polycondensation. This endcaps hydroxyl end groups with acids and avoids or reduces some side reactions that can occur to reduce MW, if not desired, when using an excess of diol or diacid from the start of a polycondensation.

Other reactive end caps (or in chain reactive groups-see later) can be introduced on the polyester prepolymers, for example via introduction of reactive double bonds, for example as in acrylate or methacrylate or itaconate or citraconate or other reactive vinylic groups. Other reactive groups (or combinations e.g. acid endcaps for reactions with epoxy functional reagents) may also be used for crosslinking and/or chain extension reactions, and which help form capsules in-situ, in the presence of the cargo.

The double bonds may be introduced via reactions of the polyester end groups with acrylic or methacrylic acid, or other acids with vinylic bonds, glycidyl methacrylate or hydroxyethyl, hydroxymethyl, hydroxypropyl, or hydroxybutyl acrylate, methacrylate, acrylamide, methacrylamide, or other functional (hydroxyl, amine, isocyanato, epoxy, acid, ester, acid chloride) acrylate, methacrylate, acrylamide or methacrylamide or other double bond containing reactive molecules.

With either acid and/or hydroxyl end groups present end-capping polyester chains with double bonds can be achieved via example reactions with glycidyl methacrylate (GlyMA). The epoxide ring (glycidyl group) can react with acid end-groups and, potentially, also with hydroxyl groups, so introducing a vinyl group to the prepolymer chain-ends. The vinyl bonds on the prepolymer can react with other similarly vinyl terminated chains in the same product mixture, and/or with differently vinyl functionalized polymers (self-crosslinking) to allow lightly cross-linking in the production of capsules—in the presence of fragrance or other cargoes. These double bonds can also react in addition reactions for example with thiols (thio-Michael reaction) or amines (aza-Michael reaction) for chain extension and/or crosslinking (see later examples). This can introduce hydrolysable or biodegradable crosslinks or branches off or between the biodegradable polyester prepolymers. Examples of typical procedures for forming capsules from prepolymers with vinyl end groups are given further below.

Thus, after end-capping—for example with varying wt. % of added glycidyl methacrylate (GlyMA)—encapsulation is progressed with in-situ cross-linking or chain extension of the polyester in the presence of cargo (e.g. fragrances or oils or waxes or butters or other lipophilic cargoes etc.) capsules.

### Example 3: End-Capped Polyester Prepolymer with Glycidyl Methacrylate (GlyMA)

Below is an example for end-capping of a polyester with glycidyl methacrylate (GlyMA) via reaction with acid end-groups (primarily, though also possibly with hydroxyl groups). The progress of the carboxylic acid—epoxy end-capping reaction can be followed by titration for acid number (AN)—a reduction showing evidence of such end-capping. This, together with analyses of residual GlyMA and also the presence of glycerol monomethacrylate (GMA a side product), both quantified by liquid chromatography (LC), enables monitoring of the end-capping reaction. Acid numbers should decrease as acid groups are increasingly end-capped with reaction with GlyMA.

GMA is the ring opened counterpart of GlyMA and is formed when unreacted GlyMA is ring opened/hydrolyzed (its level is an indicator together with polymer acid number for example, of the consumption of GMA in the end-capping reaction.)

A polyester prepolymer (see above) (SA (0.5 eq)-DA (0.25 eq)-DSA (0.25 eq)-EG (1)) was made via a bulk polycondensation, following the procedure above, from succinic acid (SA), dodecanoic acid (DA), dodecenylsuccinic acid (DSA) and ethylene glycol (EG). The polyester prepolymer had an acid number of 20 (=20 mg KOH for 1 g of polyester prepolymer product; this equates to 0.00036 moles acid per 1 g polyester; 6.000 g=0.0021 moles acid in 6 g of the prepolymer product. Then, GlyMA (0.300 g (=0.0021 mol), triethyl amine (0.027 g, 0.01 eq of polyester) and hydroquinone (0.030 g, 0.01 eq of polyester) were weighed into a 50 mL round bottom flask and placed into an oil bath attached to a condenser and vacuum with temperature at 120° C.). Once melted the reaction was maintained for 3 hrs. with rapid stirring and high vacuum. The end-capped polyester prepolymer was analyzed by liquid chromatography (LC) to determine the residual GlyMA and GMA monomer remaining. Acid number was also determined by titration. This, and other end-capped products, can then be used directly for microcapsules formation (same pot reaction) or stored to do that later if required. Crosslinking or chain extension reactions can be undertaken with the vinyl end groups now in place (see further below)—either via direct heating or with added radical initiator (oil or water soluble), during an in-situ oil in water encapsulation stage.

### Example 4: End-Capping Polyester Prepolymers with Methacrylic Acid (MAA)

The polyester prepolymer described above (194-03-1; (SA (0.5 eq)-DA (0.25 eq)-DSA (0.25 eq)-EG (1)) was also end-capped with 0.25 molar equivalents of methacrylic acid (MAA). 0.28 g of methacrylic acid was added to 3 g of the SA-DSA-DA-EG polymer previously described. To this, 14 mg (0.01 molar equivalents) of hydroquinone was added as an inhibitor. The molten polymer and methacrylic acid mixture were stirred at 120° C. for two hours to effect end-capping and so introducing a methacrylate end group.

Two other polyesters (188-34 and 188-35) containing SA at (0.95 or 0.9), DSA (0.05 or 0.1) and ethylene glycol (1.2) (thus SA-DSA-EG (0.95/0.05/1.2; and 0.9/0.1/1.2)) were similarly synthesized as previously described above. Molecular weights for the initial uncapped polyester prepolymers were 2700 gmol⁻¹ 1 and 2000 gmol⁻¹ respectively and these polymers were targeted to have—OH rich end groups (excess diol) for subsequent end capping, as described above, with methacrylate functional acid (MAA). To each prepolymer 0.1 molar equivalents of methacrylic acid was then added (50 mg to 2.4 g). 10 mg of hydroquinone was added to the reaction mixture to inhibit polymerization. The reaction mixture was heated to 120° C. and the molten polymer-MAA mixture was stirred for 3 hours. Residual MAA levels were determined by GC, respectively as: 610 ppm and 1155 ppm. Mw's increased slightly, likely due to further condensation (chain extension) at those temperatures, to 3600 gmol⁻¹ and 3000 gmol⁻¹ respectively.

Other polyester prepolymers, selected for biodegradability potential, were made via variations on these procedures in terms of diacid-diol combinations and relative ratios, reaction times and temperatures and catalyst types (all for polycondensation) and with different end-capping approaches, as shown in the examples and associated tables herein. Commercially available polyester acrylate prepolymers (or oligomers) may also be used such as those sold by Sartomer/Arkema or others.

### Example 5: Microencapsulation with Polycondensation Prepolymers—with or without Radical Polymerization Off Unsaturated End Groups

Examples of procedures for microencapsulation of a cargo such as a fragrance or an oil, using a biodegradable polyester prepolymer, optionally with reactive endcaps or in chain reactive groups, or with no reactive group, are described below. Although the steps of (a) prepolymer formation, (b) optional end-capping and (c) microencapsulation are described herein it will be evident that this sequence can be carried out in a continuous process (using the same reaction vessel throughout if desired), or as separated steps.

These first examples illustrate a process of preparing microcapsules by encapsulating a fragrance material from a prepolymer with vinyl (unsaturated) end groups (such as acrylate or methacrylate as an example) and free radical reactions of the vinyl group such as those prepared in the Examples above. Other approaches to encapsulation such as with different reactive end groups or combinations, including Michael addition reactions of the unsaturated groups (see examples later), in-chain reactive groups, or with no reactive groups (no crosslinking or chain extension), are also described further below.

### Examples 5A-5E: Microcapsule Production Examples Via Radical Reactions of Unsaturated (Vinyl) Groups, Using a Vinyl Terminated Polyester

Example 5A: One example of a microencapsulation procedure for a slurry is described for the formation of microcapsules in the presence of 30% w/w fragrance.

The polyester prepolymer (SA (0.5)-DA (0.25)-DSA (0.25)-EG (1)) with methacrylate end group (1 g), described above and fragrance (3 g; Robertet R14-3913) were homogenized in a 50 mL plastic beaker to give a clear brown (organic phase) liquid (and warmed from room temperature up to 70° C.). In a separate beaker 1.34 g of Poval (8.89 wt. %) and 4.26 g of water are stirred in presence of 0.03 g of ammonium persulfate (APS) for 10-15 min. The aqueous phase is added to the organic phase and stirred for 10-15 mins. The slurry (10 wt. % prepolymer; 30 wt. % fragrance) is then homogenized using a Silverson mixer (for small quantity) at 4200 rpm for 2 minutes. The reaction vessel is then placed in an oil bath and heated at 70-80° C. for 3 hours with stirring. In some cases, a creaming is observed. In such cases addition of 0.0075 g of Xanthan gum is added while the slurry is warm to prevent the formation of agglomerates. Microcapsules with fragrance inside were formed.

Example 5B: In another similar experiment, using the same polymer and oil phase as in Example 5A into a 50 mL plastic beaker was placed Poval (0.759 g of 11.8 wt. % solution to give a final wt. % of 1.5, water (5.211 g to give 10 g total slurry) and a radical initiator, ammonium persulfate (APS; 0.03 g, 3 wt. %). All were weighed out and then homogenized with a Silverson mixer at 4200 rpm for 30 s. The organic phase (same as above in 5A; (warmed polyester plus-fragrance) was then added to this mixture and homogenized for a further for using the same settings, prior to adding it to a 30 mL glass vial (reaction vessel) with a stirrer bar. The reaction vessel was placed into an 80° C. oil bath for 3 hours with stirring, during which capsule formation occurs with radical crosslinking of the vinyl end groups, or a portion of them.

Capsules were again readily formed and could be seen under an optical microscope. Liquid cargo was also visibly expelled upon crushing the capsules and fragrance release detected.

Example 5C: In another example using SA (0.5)-DA (0.25)-DSA (0.25)-EG (1) ENC 2188 sample) 0.25 eq GlyMA (relative to total acid in prepolymer synthesis) was used to endcap the polyester at 120° C. after 3 hours reaction under vacuum. The microcapsules then formed by the same process as just described in 5A and 5B (radical polymerization in-situ with fragrance present) could be seen under an optical microscope and were shown to contain a fragrance load of 27 wt. %. Other persulfates (potassium persulfate, KPS for example) may be used or other water soluble or redox initiators for the radical polymerization (encapsulation) stage.

Example 5D (Microcapsules ENC2223): In another set of experiments the SA (0.5)-DA (0.25)-DSA (0.25)-EG (1) (ENC 2207) polymer (ENC2207), once formed, was reacted further with added anhydride (dodecenylsuccinic, DSA, 0.1 eq; 3 hours reaction in same vessel at 120° C., with vacuum) to give acid end groups in the polyester prepolymer (AN—acid number—increases, also some polycondensation also occurs to advance MW a little) and then this prepolymer (ENC 2216) was end-capped with GlyMA (further 3 hours at 120° C. with vacuum) to give a vinyl (methacrylate) terminated polyester (ENC2211; AN decreases showing end capping with methacrylate), which was then used to make microcapsules with 30 wt. % fragrance added in to the reaction vessel. The microcapsules (ENC 2223) were formed contained 22 wt. % fragrance loading. Furthermore, within the slurry product very low levels of residual GlyMA (11 ppm) and GMA (81 ppm) were demonstrated by LC (Liquid Chromatography) analytical methods. See the Table 1 below for a summary of the steps.

TABLE 1
Examples of polyester compositions for the
microcapsules of Example 5 (MW data by GPC)

| | | | Acid Number |
|---|---|---|---|
| SAMPLE ID/DESCRIPTION | Mw/g mol⁻¹ | Mw/Mn | (AN) |
| ENC 2207 (first prepolymer): SA (0.5)-DA (0.25)-DSA | 6,400 | 2.3 | 25 |
| (0.25)-EG (1) | | | |
| ENC 2216 (modified end group of prepolymer): ENC | 9,700 | 3.4 | 44 |
| 2207 reacted with added DSA (anhydride) to add further | | | |
| acid end groups | | | |
| ENC 2211(end capped polymer): ENC2216 end-capped | 14,400 | 4.5 | 11 |
| with GlyMA (0.25 eq) - reacting acid end groups to | | | |
| introduce unsaturated end group (methacrylate) | | | |
| ENC 2223: Microcapsules made with ENC 2221 and | — | — | — |
| 30 wt. % fragrance, and with radical initiator (APS) for | | | |
| crosslinking of end groups during encapsulation | | | |

Example 5E: In another set of experiments a SA (0.7)-DA (0.3)-EG (1) prepolymer (ENC 2139), was made following a similar procedure to that described above, and was reacted with 2 wt. % GlyMA to then make another methacrylate end-capped polymer which was then used in the process (as described with APS initiator) to make microcapsules in the presence of fragrance in an oil in water process. Fragrance encapsulation was achieved at 24-32 wt. % for variously end-capped samples of this polymer (Sample IDs (respectively) ENC 2198, 2199 and 2200). Unreacted GlyMA was measured at 158 ppm and a side product, GMA, at 132 ppm—for ENC 2200 with 32 wt. % encapsulated fragrance measured inside or released from the capsules in the slurry.

Utilizing LC in the above examples for detection of GlyMA and GMA, it can be concluded that typically >99% conversion was achieved, based on residual GlyMA and GMA (glycerol monomethacrylate, the hydrolysis product of GlyMA) contents, following microcapsule preparation. These numbers are even lower when less GlyMA is used.

Further examples of other microcapsules that were similarly formed from polyester prepolymers, GlyMA end-group modified prepolymers, then crosslinked during in-situ encapsulation of fragrance via an oil in water process, included (all such prepolymers subsequently end-capped with GlyMA).

### Examples 5F: Microencapsulation with Polycondensation Prepolymers—without Radical Polymerization Off Unsaturated End Groups

Capsules were also surprisingly successfully made in some cases with prepolymers made via the same process as described above and then progressed into am oil-in-water encapsulation process but with no initiator induced crosslinking (no radical initiator used and no necessary end group modifications, and with a similar process for emulsion-encapsulation as above, after making the prepolymer) with compositions below:
Ref 2324: SA (0.5)-DA (0.4)-DSA (0.1)-cylcohexanedimethanol 1:1;
Ref 2323: SA (0.7)-Sebacic acid (0.2)-DSA (0.1)-EG (1); and
Ref 2329: SA (0.5)-DA (0.40)-DSA (0.10)-EG (1).

In some cases fragrance (as an example of an aggressive, plasticising cargo) retaining capsules can surprisingly be made without crosslinking where the pre-made polymer used for the capsule shell exhibits a melting transition (Tm— of the prepolymer), and so is semi-crystalline or has crystalline domains, and where such Tm is above ambient temperatures (>15° C. or >20° C.) and preferably above (>) 30° C., more preferably greater than 35° C. or 40° C. or 45° C. or 50° C. or 55° C. or 60° C. or greater than 60° C. or 65° C. or 70° C. or 75° C. or 80° C. or 85° C. or 90° C. or 95° C. or 100° C., as long as the polymer can still be solubilized or melt solubilized or partially solubilized in the cargo, optionally with added diluent or added compatibilising additive or polymer. In such situations, after melting the polyester and/or other polymer with the fragrance to make a melt-solution, an emulsion is then made with added aqueous phase, using processes described, and then gradually allowed to cool, so allowing some crystallization of the polymer shell around fragrance so forming a capsule shell around the fragrance without crosslinking and also still showing biodegradation or evidence of non-persistence. Crosslinking and/or chain extension and/or branching can also be applied if so desired but in some cases, it is not necessary in order to form a shell that can retain fragrance. Melting points or transitions and associated crystallinity or crystalline domains can be tailored by the conditions or rates used for cooling, the presence of additives, and/or via compositional variations in the prepolymer structure, including for example incorporation of amide or urethane bonds in the backbone of a polyester prepolymer which can aid attaining higher melting points where required or thought beneficial. Such groups can be incorporated up to a point where hot-solubilization processes for the cargo can still be completed.

The capsules once made upon cooling and/or with optional crosslinking and/or chain extensions in a slurry or dispersion may then be dried and this may aid further crystallinity development and/or create pseudo-crosslink points via hydrogen bonding. Similarly, deliberate annealing can be applied in some cases for example with controlled heating and/or cooling to aid crystalline domain formation in the capsule shell, in some cases—and they may then be re-dispersed in water as necessary. Drying may be accomplished by simple air drying, controlled air-heating, drying under vacuum, or via spray drying or other known drying processes for particles capsules. Such capsules may be also coated with an outer layer (same or different material to the shell material) during, or after, such drying.

The ability to encapsulate a variety of lipophilic cargoes (not just fragrances, which are known to be among more plasticizing or aggressive of cargoes) in uncrosslinked and in crosslinked polyesters allows a tailoring of biodegradability for different cargoes and/or for different end use formulations (also some less aggressive than others in terms of their propensity to attack polyester shell walls). For the more demanding of end use formulations and/or cargoes some crosslinking and/or some crystalline domains in the polyester shell may be required for suitability in end uses, while still retaining some biodegradability properties.

It is recognized that fragrances, natural or essential oils and other lipophilic cargoes have different propensities to solvating or attacking polymer shell walls and also that many are typically mixtures of various chemical components and these components and their ratios in products differ from grade to grade or from product to product. Consequently, the design of the polyester prepolymer composition, its crystallinity (where present), and/or its degree of crosslinking may be required to be adjusted for a particular fragrance or other lipophilic or oil/diluent solubilized cargo, in some cases, so as to allow solubility or miscibility or compatibility of the polyester prepolymer with the cargo when hot or warmed—but being transformed into insoluble solid capsule shell walls when cooled (ambient) or when, otherwise, the encapsulation transformation process has been completed. The attainment of insolubility or capsule wall formation/solidification may thus be via formation of crystalline or other insoluble solid domains, and/or crosslinking and/or pseudo crosslinks such as via hydrogen bonding (through amides or urethanes which can be co-incorporated for example) and/or via addition of di- or multi-valent bases such as calcium or magnesium oxide that complex with acid groups of the polyester, or via in-situ chain extension that builds up molecular weight or links polymers or oligomers.

Microcapsules were typically imaged by optical microscopy before and after crushing a dilute dispersion with a glass slide. Images before/after crushing look significantly different; uncrushed are circular; crushed show complete deformation from circles and indicate the release of fragrance/oil cargo. Thus, optical microscopy showed the presence of capsules that could be crushed to release the fragrance from these polyester capsules.

### Example 6: Polyester Prepolymers with In-Chain Reactive Groups (Example: Itaconate Polymers)

As an alternative to end-capped prepolymers, polyesters were made with unsaturated diacids or diols and so can be used directly (no end-capping) for making crosslinked capsules. Examples of unsaturated diacids useful in such approaches are maleic, fumaric, itaconic (IA), citraconic, and others.

For sample referenced as RD 201-16: Succinic acid (SA; 0.425 mol eq, 12.74 g), itaconic acid (IA; 0.075 mol eq, 2.48 g), 1,6 hexanediol (HD; 0.5 mol. eq, 15.000 g), hydroquinone (50 mg) and butyl stannoic acid (Fastcat 4100; 0.003 mol. eq, 70 mg) were added to a 250 mL round bottom flask and melted with stirring at 160° C. attached to a condenser and vacuum pump. Once melted, the vacuum was increased slowly over several hours to draw off the water produced by the step polycondensation polymerization (a minimum of 100 psi). The reaction was continued for 6-24 hours depending on targeted molecular weight (MW). MWD was determined by SEC (THF mobile phase, relative to polystyrene standards) yielding an Mw of 54.7 Kgmol⁻¹ and D=3.2 after 24 hours, Tg and Tm were determined by DSC (2 heating/cooling cycles, −80° C. to 100° C., 10° C./min), yielding values of −47.8° C. and 37.5° C. for Tg and Tm respectively (for RD201-16; SA (0.85)-IA (0.15)/1,6 HD (1.0)). For a similar polymer prepared with a composition of: SA (0.95)-IA (0.05)/1,6 HD (1.0) (ref RD201-13) a Mw of 54.4 Kgmol⁻¹ was achieved after 24 hours at 160° C. and Tm was determined by DSC, as 44.7° C. A range of variations with different itaconate contents and also with different diols and/or other diacids (or their derivatives) can be made to tailor double bond contents and/or the balance of hydrophobicity-hydrophilicity of the chain structure. Examples of some further polymers synthesized include (all catalyzed by butyl stannoic acid and 160° C. (polycondensation) either for 24 or 8 hours) are given further below.

Example 7: Radically Polymerized Prepolymers with in chain unsaturation for Microencapsulations—with Itaconate functional Polyesters (similar concepts will also apply for maleate, fumarate, citraconate (and pendant in-chain acrylate or methacrylate and the like). Capsule formation with free radical crosslinking and/or chain extension with itaconate polyester with an oil soluble initiator is described below as one example of such an approach:

1 g of polyester prepolymer (201-16-1: SA (0.85)-IA (0.15)/1,6 HD 1/1—prepared as above in Example 6) was melted into 4 g fragrance at 60° C., agitating to mix. 50 mg Vazo 67 (an oil soluble radical initiator) was added and allowed to dissolve. To this oil or organic phase an aqueous phase was added: 1 g of 10% Poval solution in water/5% HMHEC (Natrosol 330 plus CS)/10% PVP K120 was added and the mixture was homogenized at 16,000 s−1 with an IKA Ultraturrax for 30 seconds. During homogenization, 7.3 g of 1% Poval 40-88, heated to 50° C. was slowly added. The resulting emulsion was homogenized for 120 seconds. The emulsion was subsequently heated at 80° C. for 2 hours, with stirring and allowed to cool slowly. Microcapsules with fragrance as a core were formed. A hexane wash removed either free or weakly adsorbed oil/cargo for determination of encapsulated and free fragrance amounts.

A similar procedure was used with other polyesters, for example:
Ref 201-13: 0.95SA-0.05 IA/1.00 HDD;
Ref 201-10: 0.85 SA-0.1-DSA-0.05 IA/1.00 HDD, and
Ref 201-22: 0.75 SA-0.25 IA/1.00 HDD, and others.

### Example 8: Polyester Microcapsule Shells with Unsaturation (Double Bonds Such as Methacrylate Acrylate or Itaconate Etc) Made without Added Free Radical Initiator or Co-Reactant Crosslinking

1 g of polyester 201-16-1 (SA (0.85)-IA (0.15)/1,6 HD (overall diacid:diol=1:1) was melted into 4 g fragrance at 60° C., agitating to mix. To this, 1 g of 10% Poval solution in water/5% HMHEC (Natrosol™ 330 plus CS)/10% polyvinylpyrrolidone (PVP K120) was added as stabilizer and the mixture was homogenized at 16,000 s−1 with an IKA Ultraturrax™ for 30 seconds. During homogenization, 7.3 g of 1% Poval 40-88, heated to 50° C. was slowly added to the fragrance-polymer melted mix (at ˜60° C.). The resulting emulsion was homogenized for 120 seconds. The emulsion was allowed to cool slowly with stirring. Microcapsules with fragrance as a core were formed.

Note: no radical initiator is used in this example and capsules were still formed readily via this straightforward process and which retained fragrance as is. A similar procedure was used with other polyesters, for example, 201-13 (0.95 SA/0.05 IA/1.00 HD) and 201-10 (0.85 SA/0.1 DSA/0.05 IA/1.00 HD). While not been constrained by theory it is possible some self-reaction of unsaturated groups, in-chain and/or as end groups, occurs to progress some chain extension or branching or light crosslinking and aid encapsulation during that stage of the process, and may aid capsule robustness for some cargoes in such cases. Crystallinity may also be present and be facilitating this effect.

Variations include for example the use of added hydrophobically modified silica (HM silica) which is added with PVP K 120 and the polyester to which is then added a water-POVAL solution together with fragrance with homogenization of the whole mixture—again with no initiator induced crosslinking step. This was used for a polymer, 201-13-1, with a composition of: 0.95 SA/0.05 IA/1.00 HD.

Other microcapsules were similarly prepared from polyester prepolymers with unsaturated groups, with and without added radical initiators present, but with some variations and comments noted below. Examples are:
201-30-3: SA(0.75)-IA (0.25)-1,12 DDO (1.0), prepolymer was prepared as described and then Poval was added after prepolymer is made, stirred 80° C./2 hrs (without initiator).
201-30-4: as above with 201-30-3 but with added Vazo 67 radical initiator (2 hrs/80° C.). Molecular weight was noticeably increased (GPC in THF of dissolved capsule shells) yet crosslinking or extension reactions were not of a high level since all material was soluble in THF for GPC analyses. Fragrance retention was superior for the 201-30-4 capsules (which has added radical initiator) when compared to the analogous capsules (201-30-3) above made without initiator—though biodegradation was slightly lower in comparison, though still evident.
196-52-1, 196-53-1, 196-54-1: capsules were made following the same procedure starting with polyester the prepolymer 201-17-1 (SA (0.75)-IA (0.25)-1,8 Octanediol (1.0)) with added PVP (K120 as supplied by Ashland LLC) and POVAL and Sipernat 50S Stabilizer (40-88)—capsules were made with R14-3913 fragrance cargo).

Further examples of polymer compositions used to successfully make microcapsules (ENC) with fragrance as a cargo, are tabulated below.

TABLE 2
Examples of polyester prepolymer compositions used in various micro
encapsulations in the examples described or in the table, and/or in other tables further below

| | | | | | | | Polymer | |
|---|---|---|---|---|---|---|---|---|
| | | | | Polycondensation | | | Molecular | |
| | | | | (prepolymer) | | | Weights (Mw | Tm (° C.) |
| | | | ratio | Reaction Conditions | | | (g/mol))- before | (DSC)- |
| Sample References | | | [diacid]/ | Temperature | | | capsule | where |
| RD/ENC | | Polyester composition | [diol] (eq) | (° C.)/Time Hrs | | Catalyst | formation | measured |
| 201-01 | 2289 | SA(0.9)-DSA(0.1)/EG | 1.1:1 | 190 | 24 | BSA | 47,400 | NM |
| 201-02 | 2290 | SA(0.9)-DSA(0.1)/1,4 | 1.1:1 | 170 | 24 | BSA | 16,200 | 104.4 |
| | | BDO | | | | | | |
| 201-03 | 2310 | SA(0.9)-DSA(0.1)/1,4 | 1.1:1 | 190 | 24 | BSA | 24,200 | 104.4 |
| | | BDO | | | | | | |
| 201-04 | 2311 | SA(0.9)-DSA(0.1)/1,6 | 1.1:1 | 190 | 24 | BSA | 66,500 | 42.8 |
| | | HD | | | | | | |
| 201-05 | 2312 | SA(0.9)-DSA(0.1)/1,12 | 1.1:1 | 190 | 24 | BSA | 192,000 | 67.2 |
| | | DDD | | | | | | |
| 201-07 | 2325 | SA(0.9)-DSA(0.1)/1,6 | 1:1 | 190 | 24 | BSA | 120,000 | 42.0 |
| | | HD | | | | | | |
| 201-08 | 2333 | SA(0.8)-DSA(0.15)-IA | 1:1 | 160 | 24 | BSA | 25,100 | NM |
| | | (0.05)/EG | | | | | | |
| 201-09 | 2334 | SA(0.8)-DSA(0.15)-IA | 1:1 | 160 | 24 | BSA | 65,000 | 32.9 |
| | | (0.05)/1,6 HD | | | | | | |
| 201-10 | 2336 | SA(0.85)-DSA(0.1)-IA | 1:1 | 160 | 24 | BSA | 49,000 | 40.6 |
| | | (0.05)/1,6 HD | | | | | | |
| 201-11 | 2337 | SA(0.85)-DSA(0.1)-IA | 1:1 | 160 | 24 | BSA | 10,900 | 95.6 |
| | | (0.05)/1,4 BDO | | | | | | |
| 201-12 | 2338 | SA(0.85)-DSA(0.1)-IA | 1:1 | 160 | 24 | BSA | 22,000 | NM |
| | | (0.05)/1,4 BDO-1,6 HD | | | | | | |
| | | (0.5-0.5) | | | | | | |
| 201-13 | 2341 | SA(0.95)-IA (0.05)/1,6 | 1:1 | 160 | 24 | BSA | 54,400 | 44.7 |
| | | HD | | | | | | |
| 201-16 | 2345 | SA(0.85)-IA (0.15)/1,6 | 1:1 | 160 | 24 | BSA | 54,700 | 37.5 |
| | | HD | | | | | | |
| 201-17 | 2346 | SA(0.85)-IA (0.15)/1,8 | 1:1 | 160 | 24 | BSA | 79,100 | 52.6 |
| | | octanediol | | | | | | |
| 201-18 | 2347 | SA(0.85)-IA (0.15)/1,5 | 1:1 | 160 | 24 | BSA | 69,600 | NM |
| | | pentanediol | | | | | | |
| 201-21 | 2356 | SA(0.5)-IA (0.5)/1,6 HD | 1:1 | 160 | 24 | BSA | 15,700 | NM- |
| 201-22 | 2357 | SA(0.75)-IA (0.25)/1,6 | 1:1 | 160 | 24 | BSA | 145,000 | 30.3 |
| | | HD | | | | | | |
| 201-27-1 | 2370 | SA(0.75)-IA (0.25)/1,6 | 1:1 | 160 | 8 | BSA | 33,400 | 37.8 |
| | | HD | | | | | | |
| 201-28-1 | 2370 | SA(0.75)-IA (0.25)/1,12 | 1:1 | 160 | 8 | BSA | 24,200 | 65.0 |
| | | DDO | | | | | | |

TABLE 3
Examples of microencapsulation experiments with parent polyester prepolymer
compositions with or without reactive end group functionality

| | | | | | | | % |
|---|---|---|---|---|---|---|---|
| | | Parent | | | | Fragrance | Encapsulation |
| | | polymer | | | | load % | efficiency (%) |
| | | REF | | | Added | weight for | of cargo |
| SAMPLE REF | | (Modified, | Parent polymer | End group | radical | encapsulation | retained/ |
| ENC/RD | | unmodified) | chemistry | modification | initiator | stage | encapsulated) |
| ENC | 192-33-1 | 192-28-1 | SA(0.5)-DA(0.4)- | None | None | 31 | 87.9 |
| 2295 | | | DSA(0.1)- EG 1:1 | | | | |
| ENC | 192-35-1 | 192-34-1, | SA(0.6)-Sebacic acid | GlyMA | APS | 31 | 83.9 |
| 2303 | | 192-23-1 | (0.2)-DSA (0.3)-EG 1.1:1 | | | | |
| ENC | 192-35-2 | 192-34-2, | SA(0.6)-Sebacic acid | GlyMA | APS | 31 | 77.8 |
| 2304 | | 192-25-1 | (0.4)-DSA (0.1)-EG 1.1:1 | | | | |
| ENC | 192-35-3 | 192-34-3, | SA(0.5)-DA (0.4)-DSA | GlyMA | APS | 31 | 69.5 |
| 2305 | | 192-28-1 | (0.1)-EG 1:1 | | | | |
| ENC | 192-40-1 | 192-37-1 | SA(0.5)-DA (0.4)-DSA | None | None | 31 | 55.1 |
| 2324 | | | (0.1)- | | | | |
| | | | cylcohexanedimethanol | | | | |
| | | | 1:1 | | | | |
| | 188-73-1 | 188-39-1 | SA-BDDA (1:1) | GlyMA | APS | 29 | 86 |
| | 188-93-1 | 188-83-1 | SA(0.5)-DSA(0.5)- | MAA | APS | 22 | 87 |
| | | | EG(1.2) | | | | |
| | 194-08-2 | 194-03-1 | SA(0.5)-DA(0.25)- | GlyMA | APS | 27 | 83 |
| | | | DSA(0.25)-EG 1:1 | | | | |

TABLE 4
Examples of microencapsulation experiments with polyester compositions with
in- chain reactive end group functionality using parent prepolymers, some from Table 2,
some with repeats or variations of conditions or additives

| | | | | | | % fragrance | |
|---|---|---|---|---|---|---|---|
| | Parent | | | Added | | used in | |
| Encapsulation | Polymer | Polymer | End group | radical | Other | encapsulation | % efficiency |
| ID | ID | Composition | modification | initiator | comments | step | encapsulation |
| 201-32-1 | 201-09-1 | SA(0.8)-DSA(0.15)- | None | None | PVA (44:88) | 31 | 55 |
| | | IA(0.05)/1,6 HD 1:1 | | | stabilizer | | |
| 201-32-2 | 201-28-1 | SA(0.75)-IA (0.25)- | None | None | PVA (44:88) | 31 | 55 |
| | | 1,12 Dodecanediol | | | stabilizer | | |
| | | 1:1 | | | | | |
| 201-32-3 | 201-28-1 | SA(0.75)-IA (0.25)- | None | V67 | PVA (44:88) | 31 | 60 |
| | | 1,12 Dodecanediol | | | stabilizer | | |
| | | 1:1 | | | | | |
| 196-56-1 | 201-17-1 | SA(0.85)-IA (0.15)- | None | None | PVP K- | 23 | 78 |
| | | 1,8 Octanediol 1:1 | | | 120/PVA | | |
| | | | | | (44:88) with | | |
| | | | | | Sipernat S50 | | |
| | | | | | dispersing aid | | |
| 196-56-2 | 201-17-1 | SA(0.85)-IA (0.15)- | None | None | PVP K- | 22 | 57 |
| | | 1,8 Octanediol 1:1 | | | 120/PVA | | |
| | | | | | (44:88) with | | |
| | | | | | Sipernat S50 | | |
| | | | | | dispersing aid | | |
| 196-42-1 | 201-13-1 | SA (0.95)-IA (0.05)- | None | None | PVP K- | 25 | 51 |
| | | 1,6 Hexanediol (1:1) | | | 120/PVA | | |
| | | | | | (44:88) with | | |
| | | | | | Sipernat S50 | | |
| | | | | | dispersing aid | | |
| 196-42-2 | 201-13-1 | SA (0.95)-IA (0.05)- | None | None | PVP K- | 33 | NM |
| | | 1,6 Hexanediol (1:1 | | | 120/PVA | | |
| | | | | | (44:88) with | | |
| | | | | | Sipernat S50 | | |
| | | | | | dispersing aid | | |
| 201-20-1 | 201-16-1 | SA(0.85)-IA (0.15)- | None | V67 | PVP K120 PE | 31 | NM |
| (ENC 2349) | | 1,6 HD 1:1 | | | stabilizer | | |
| 201-23-1 | 201-22-1 | SA(0.75)-IA (0.25)- | None | V67 | PVA (44:88) | 31 | 59 |
| (ENC 2358) | | 1,6 HD 1:1 | | | stabilizer | | |
| 201-26-1 | 201-22-1 | SA(0.75)-IA (0.25)- | None | V67 | PVA (44:88) | 31 | 63 |
| (ENC 2366) | | 1,6 HD 1:1 | | | stabilizer, scale- | | |
| | | | | | up of 201-23-1 | | |
| 201-26-2 | 201-22-1 | SA(0.75)-IA (0.25)- | None | None | PVA (44:88) | 31 | 55 |
| (ENC 2367) | | 1,6 HD 1:1 | | | stabilizer, scale- | | |
| | | | | | up of 201-23-3 | | |

Notes
DA or DDA—dodecanedioic acid, DSA—dodecenyl succinic anhydride, OSA—octenyl succinic anhydride, SA—succinic acid, LGA—D,L-lactide -Glycolide, EG—ethylene glycol, p-TSA—para-toluene sulfonic acid, HD—1,6 hexanediol, APS—ammonium persulphate (water soluble radical initiator, V 67—azo 67 (oil soluble radical initiator), BDO—1,4-butanediol, IA—itaconic acid, DDD—dodecanol, GlyMa—glycidyl methacrylate, GMA—glyceryl methacrylate, PVA—polyvinyl alcohol, N330—Natrosol ™M 330 + HMHEC (hydrophobically modified hydroxyethyl cellulose) BSA—Butyl stannoic acid, NM—not measured.

### Example 9: Capsule Formation with Crosslinking and/or Chain Extension with Itaconate Polyester (Prepolymer) Via Addition Reactions During In-Situ Polymerization-Encapsulation (Alternative to Radical Crosslinking, Grafting or Branching, or for Chain Extensions)

In addition to free radical crosslinking of the itaconate groups, or other double bonds such as acrylate or methacrylate end groups, as described above, during the emulsion-encapsulation stage, the itaconate (or other double or triple bond containing polyesters) can be crosslinked, or branched, or chain extended, and/or side-chain (pendant) functionalized, via radical methods, or via addition reactions depending on the double bond contents and stoichiometries with added co-reagents. Such addition reactions can proceed with added nucleophiles (also known as donors) such as thiols, or amines. For example, a monofunctional thiol (or a secondary amine) will add to produce a pendant side chain off the double bonds. This can be advantageous to further tune hydrophobicity—hydrophilicity and/or to aid deposition of capsules onto (or affinity with) surfaces or materials such as clothing materials (laundry applications), surfaces to be cleaned with household cleaners, or hair or skin, among others. A di- or multi-functional thiol (or a primary amine or a difunctional secondary amine or a multifunctional primary or secondary amine) will form crosslinks or branches off the double bonds, and the relative degrees of linear chain extended polymer or branched polymer or crosslinked polymer domains will be determined by the number of itaconate (or other in/off chain) double bonds per polyester and the functionality of the donor (amine or thiol). Design of such ratios can lead to in-situ (in presence of the cargo for encapsulation stage) chain extension as the prevalent resulting change to the prepolymer, and this can make higher molecular weight linear or largely linear or grafted polymer shells off the double bonds if they are at, or near, the polyester chain ends and/or grafted polymer shells if not just at chain ends. Higher functional thiols or amines can be used to introduce branching and/or crosslinking. This approach can lead to relatively either low or relatively highly crosslinked capsule shell wall structures to ensure capture and/or retention of plasticizing of volatile cargoes and still surprisingly retain evidence of biodegradability.

The levels of crosslinking at which this occurs will vary according to the backbone chain structure, crosslinking approach/mechanism (including ant other reagents added for enhancing or limiting crosslinking), and the nature of the fragrance and other components potentially present.

Various mixed approaches of these examples are of course feasible. In particular, mixtures or blends (made deliberately in-situ, and/or via post-synthesis blending or mixing) of linear and crosslinked chains may be formed in the capsule shell making process so enabling further tailoring of biodegradability. Or, use of monofunctional (pendant functionality) with multifunctional added reagents (for controlled crosslinking) to tailor hydrophobic balance and/or deposition performance.

The use of addition reactions, for example Michael additions, on double (or triple) bonds (whether (meth)acrylate, itaconate, citraconate, maleate, fumarate, maleimide, or others) in a prepolymer such as a polyester or oligo-ester, with added thiol or amine reagents, can produce lightly or more highly crosslinked systems to enable biodegradability to be retained in the crosslinked system. For example, thiols or amines, which may be hydrophobic or hydrophilic may be used to crosslink polyesters with suitable double or triple bond functionality. These unsaturated bonds when reacted in such addition reactions, for example with thiols (thio-Michael reaction) or amines (aza-Michael reaction) for chain extension, and/or branching and/or crosslinking can introduce hydrolysable or biodegradable crosslinks or branches off or between the biodegradable polyester prepolymers functionalized with in chain and/or chain end unsaturation. The use of hydrophilic reagents (e.g. with PEG (polyethylene glycol) or other hydrophilic chains) to crosslink the double bonds in the polyesters so produces a hydrophilic environment around the crosslink bonds, further facilitating their hydrolysis or biodegradation. Other chains which are readily degradable or hydrolysable with thiol or amine groups can be used as crosslinkers of the polyester or other prepolymers (melted in, or dissolved in, the fragrance; then emulsified or dispersed, and encapsulated).

1 g of the polyester prepolymer, 201-27-1 (SA (0.75)-IA(0.25)/1,6 HD (overall diacid: diol=1:1) was melted into 4 g fragrance at 60° C., agitating to mix. To this, 1 g of 10% Poval solution in water/5% HMHEC (Natrosol™ 330 plus CS)/10% polyvinylpyrrolidone (PVP K120) was added and the mixture was homogenized at 16,000 s⁻¹ with an IKA Ultraturrax™ for 30 seconds. During homogenization, 7.3 g of 1% Poval 40-88, heated to 50° C. was slowly added to the fragrance-polymer melted mix (at ˜60° C.). This was held at 60° C. with stirring and the pH of the continuous phase was adjusted to 9. To this, 90 mg of 2,2′-(ethylenedioxy) diethanethiol (0.5 molar eq. on itaconate) was added and the mixture was allowed to stir at 60° C. for 2 hours, followed by slow cooling. Microcapsules in a slurry which contained fragrance were formed.

Other thiols can be used. Amines or diamines may also be used in place of thiols in these Michael Addition reactions for crosslinking, branching and/or chain extensions. Other conjugated or activated carbon double bonds can also be used in place of itaconate. When this approach is used for crosslinking it creates ‘spacers’ between crosslinks and, also, creates hydrolysable bonds at the crosslink points—an approach which helps to control the effects of crosslinking on biodegradation, yet enabling retention of the cargo in the capsule shell. Other approaches to introducing hydrolysable crosslinking points or chain extensions or branching are described further below (for example via epoxy-acid or anhydride reactions).

### Example 10: Encapsulation Using Other Chain Extension Reactions on a Polyester

A 250 mL flask equipped with stirrer and condenser with vacuum connection and collecting vessel was charged with 10 g (0.0846 moles) of succinic acid (˜20% excess in moles compared to the diol), 4.376 g (0.0705 moles) of ethylene glycol, 0.02 g of sulfuric acid as catalyst, and the mixture was heated gradually to an internal temperature of 120° C. while stirring under reduced pressure of 10 mbar and kept it reacting for 120 minutes (Ref ENC-1673).

10.163 g of the reaction mixture (as is) of poly(succinate) ethylene glycol (PSA-EG. MW 2800 g/mol), was transferred in a 100 mL glass vial equipped with magnetic stirrer bar and then was charged with 13 g of triacetin, 6.5 g of Waglinol oil and 6.5 g of spring garden fragrance. Diglycidyl glycerol (DGE) ether was added afterwards at room temperature. The addition of DGE was to perform in-situ chain extension (likely also with branching and/or light crosslinking) via formation of further ester (hydroxy-ester) groups from the acid chain ends of the PSA-EG backbone by reacting carboxylic acid end groups with the epoxide groups of DGE, in the process of forming the capsule shell wall (encapsulation). 78 mL of water were introduced into the flask and the slurry was homogenized by an IKA homogenizer at 4000 rpm. The reaction mixture was heated gradually to an internal temperature of 80° C. and triethylamine (TEA, a drop) catalyst was added with magnetic stirring at 80° C. for 2.5 hours. The slurry product (ENC 1707) was centrifuged and left to air dry overnight in an operating fume cupboard. Capsules of 5-10 microns were clearly formed and when pressed under a microscope slide clear release of a cargo was seen which was determined as attaining a 50-60% retention of the input of fragrance-Waglinol mixture. Other difunctional epoxides or multifunctional epoxides, including the epoxides of vegetable oils, (such as epoxidized soya bean oil which can also introduce branching or crosslinking to tailor biodegradation—encapsulation efficiency and retention), may be similarly used in reactions with acid or anhydride functional polyester prepolymers. Chain extensions or branching or crosslinking is affected by the epoxy—acid (or anhydride) reaction and creates a hydroxy-ester link as a hydrolysable (degradable) branch point or crosslink, off or with the biodegradable polyester prepolymer. A higher proportion of acid end groups can be introduced via reaction of the polyester prepolymer with an acid or anhydride (for example succinyl or maleic anhydrides, or acids) at the end of the initial polyester formation reaction (or via a slight excess of diacid in the stoichiometry of that initial polyester formation reaction). Similarly, an anhydride or acid functional vegetable oil or another precursor can be reacted with an epoxy-, or hydroxyl-, functional polyester prepolymer. Further routes to hydrolysable (degradable) crosslinks for biodegradable polyester prepolymers, also compatible with the cargo are described above, for example via Michael additions.

### Example 11: Analogous Fragrance-Free Capsules (for Biodegradation Tests)

For biodegradability tests, analogous fragrance-free polyester particles were synthesized to eliminate any influence on the final biodegradability result by the fragrance oil, components of which can be classed as readily biodegradable. As one example, 1 g of polymer was melted at 60° C. Vazo™ 67 was added to the molten polymer at 60° C. alongside 1 g of Poval/HMHEC mixture was homogenized at 16,000 s⁻¹ with an IKA Ultraturrax™ for 30 seconds. During homogenization, 7.3 g of 1% Poval 40-88, heated to 50° C. was slowly added. The resulting emulsion was homogenized for 120 seconds. The emulsion was subsequently heated at 80° C. for 2 hours, with stirring and allowed to cool slowly. All biodegradation tests are conducted via OECD, or ASTM, or ISO, or EN or related methods, sometimes running for longer time periods, and always with a reference compound (as stipulated in the test method used) to ensure conformance of the test conditions.

Biodegradation tests can also be undertaken on capsules with a known inert (silicone oil for example) reference cargo or the actual fragrance cargo but also running a blank with that cargo at the measured encapsulated level (wherein the two BOD results subtracted accordingly, and the concentration adjusted to that of the capsule shell).

### Example 12: Biodegradation Testing

This was carried usually out according to OECD methods. For example, methods such as OECD 301D, 301F, 302B, 306, were variously used, some over extended timelines. Samples that are insoluble in aqueous media often require development for a suitable dispersion or form for the test. In some cases, the EN 14852:2018 or EN ISO 14851:2004 test can be used, which runs for time period of 6 months in aquatic media (and is also cited, along with others such as those above, in ECHA draft protocols for avoidance of microplastics concerns). Innocula and suitable water (secondary effluent surface water, seawater or activated sludge) were used as supplied from a local sources such as a wastewater treatment plant. A mineral medium specified by the OECD 301D method and the inoculum were added to deionized water which was subsequently aerated for 20 minutes prior to addition of the sample polymer sample at a concentration of 4-10 mg/ml depending on predicted biodegradability.

In some examples, biodegradation was monitored from measurements of dissolved oxygen content. In some examples this test is done in fresh water using inoculum supplied by a local water treatment plant. This test is used to mimic the environment these polymers will be in after going through a freshwater waste treatment plant. This test uses a readily biodegradable sodium benzoate reference as a positive control. All samples are run in duplicate. Measurements were taken approximately at 7 day intervals to at least 28 days and in many cases beyond. Example data is given in the table below. For example, the polyester (SA (0.5)-DA (0.25)-DSDA (0.25)-EG (1.0); ENC 2188) was measured to be 16% biodegradable after 28 days but is clearly increasing over time to double that by day 120 indicating reasonable evidence for non-persistence in the environment. The table below provides a summary of some example polyesters tested for biodegradability in aquatic environments (OECD 301D, surface water/secondary effluent). Such polymers can be used for encapsulation of fragrances and the like, according to one or more of the processes described herein. Biodegradation levels in activated sludge for example in OECD 301F testing, would be expected to be greater and/or more rapid in all cases.

TABLE 5
Biodegradability measurements of Example capsule shells (301D surface water)

| | | Biodegradation | | |
|---|---|---|---|---|
| ID/REF | Description | Day 28 | Day 60 | Day 120 |
| ENC 2198 | SA(0.5)-DA(0.25)-DSA(0.25)-EG(1). | 16 | 20 | 27 |
| 188-83-1 | SA(0.5)-DSA (0.5)- EG (1.2) | 16 | 21 | 33 |
| 188-86-1 | DSA (1)-EG (1.2) | 0 | 2 | 10 |
| 183-83-1 | SA:DSA:EG 0.5:0.5:1.2 | 18 | 21 | 33 |
| ENC 2096 | SA (0.6) - DSA (0.4) - EG (1.2) | 17 | 21 | 29 |
| 192-32-4/ENC | SA(0.6)- Sebacic acid (0.2)-DSA (0.3)/EG (1) | 22 | 24 | — |
| 2294 | | | | |
| 201-15-3 | SA (0.85)- DSA (0.1)- IA- (0.05)/1,6 Hexanediol (1), with | 9 | 16 | — |
| | initiator (Vazo ™ 67) - crosslinked | | | |
| 201-15-2 | Polyester SA (0.85)- DSA (0.1)- IA- (0.05)/1,6 Hexanediol | 21 | 47 | — |
| | (1), no initiator (no crosslinking) | | | |

Note 188-86-1 is included here as an example of a polyester which would not meet criteria of inherent or primary inherent biodegradability and so not showing sufficient evidence in this test for being biodegradable, since its biodegradation had not started by day 28. Nevertheless, it can be seen that it does biodegrade thereafter and so may reasonably be expected to be non-persistent, although more testing would typically be required to support that. Testing for longer times, and/or in activated sludge or soil or compost could be applied. While not being bound by theory that polyester is a particularly highly hydrophobic polyester with its high alky side chain (DSA) content.

Biodegradability can also be tailored, as can fragrance compatibility, by adjusting the hydrophilic-hydrophobic balance among other things (including crosslinking levels). If a very highly hydrophobic polyester is tested (for example, a polymer made from solely DSA-EG (1:1.2, 188-86-1, ENC 2110) and which has a relatively high overall content of a hydrophobic group such as a long alkyl pendant chain (for example from DSA, dodecenylsuccinic acid)) on every ester repeat unit) a low biodegradation rate (2% after 60 days OECD 301D, surface water) is shown—but biodegradation is still increasing (10% at 120 days and increasing) and so in surface water is more slowly biodegrading but may still be able to be described as non-persistent over or after a longer time and/or in different test media e.g. compost or activate sludge. This allows control of biodegradation rates. Whereas substituting some of the DSA, a highly hydrophobic diacid, with a short chain diacid such as succinic (SA) with no pendant alkyl groups (significantly less hydrophobic) results in a higher degradation rate in this aquatic media—freshwater—likely much higher in activated sludge or compost. A polyester with a composition of DSA(0.5)-SA(0.5)-EG (1.2) for example showed a biodegradation level of 21% at 60 days; 33% at 120 days; 188-83-1, ENC 2105 Intermediate levels of biodegradation are able to be tailored by varying DSA (or OSA octenyl succinic acid/anhydride, and the like)—SA ratios and/or via use of longer chain or shorter or medium chain diacids and/or diols. Similarly, fragrance or other cargo compatibility can be tailored (more of the hydrophobic alkyl side chain resulting in greater compatibility with fragrance).

It is also worth noting that a small or controlled degree of crosslinking will have an effect to reduce or slow down on biodegradation, though still showing biodegradability or evidence of non-persistence over time, while adding robustness to the capsule which may be helpful for some cargo encapsulations. 201-15-3 (SA (0.85)-DSA(0.1)-IA-(0.05), a particle dispersion from a polymer containing itaconic acid (IA) which was subsequently heated to 80° C. in the presence of an oil soluble radical initiator Vazo™ 67 (which crosslinks or chain extends the itaconate unsaturated groups) shows less biodegradation when compared to the analogue (201-15-2, same polymer) wherein the particles were prepared in the same way but in the absence of a radical initiator. Nevertheless, the crosslinked sample surprisingly still shows biodegradability over time in such mild aquatic conditions. FIG. 17 illustrates the relative biodegradation plots.

Thus, there is balance and for some applications where a low, but perceptibly ongoing, biodegradation rate can be accepted, and where combined with for example a higher fragrance or other lipophilic cargo compatibility and/or higher water resistance, can still make a desirable end product which will be non-persistent.

2. Prepolymer Route (Polyester Made by Ring Opening Polymerization for Example PLGA Polymers)

Further examples of the invention are described where microcapsules with a lipophilic core and a biodegradable polymeric shell are made by: —making an oil-in-water emulsion of an oil phase comprising an aliphatic polyester polymer or prepolymer comprising lactide and/or glycolide units such as are made by ring opening polymerization, and a lipophilic cargo, optionally with added diluent or solvent and/or aided by application of heat, —optionally adding a catalyst or initiator to one phase, —forming the capsule shell wall either by cooling or by an in-situ reaction of the prepolymer or polymer, and obtaining the cargo encapsulated microcapsule.

The diluent will preferably be a solvent for the PLGA or related polymer when warmed at moderate temperatures such as below 100° C., 90° C. or less than 50° C. or at room temperature, and will preferably have some miscibility with water, and will be, for example, an alkylene carbonate such as ethyl carbonate or propylene carbonate, acetone, dimethyl sulfoxide, a hydroxy acid or hydroxy ester such as methyl or ethyl or butyl lactate or an ester with some water miscibility such as triacetin.

Where the PLGA polymer or prepolymer contains unsaturated reactive groups the initiator is preferably a radical initiator which may be a peroxide or an azo based radical initiator or a redox system such as a persulfate based system or maybe a photoinitiator for UV induced radical reactions.

### Example 13: Polyester Polymers/Prepolymers Made Via Ring Opening Polymerization (ROP) for Subsequent Microencapsulations (Encapsulation Stage Described in Example 13)

Ring opening polymerization is another route making polyesters. Although more limited (compared to polycondensation) in the choice of starting reagents (monomers) it can build molecular weight more readily and with shorter reaction times to do so.

### Example 13A: EG Initiated ROP

5 g (34.7 mmol) D,L-Lactide, 4.03 g (34.7 mmol) glycolide, 0.028 g (0.07 mmol) stannous octoate and 0.0011 g (0.017 mmol) ethylene glycol (EG) were charged to a dry flask with an added condenser. The mixture was held under nitrogen and gradually heated to 150° C. The reaction mixture was held at 150° C. for two hours to produce a viscous amber liquid. This was diluted in THF, precipitated in methanol and dried under vacuum to isolate a PLGA polymer with 50/50 mole ratio composition (ref 192-13-1).

### Example 13B: HEMA Initiated ROP of PLGA (HEMA Functionalized PLGA)

5 g (34.7 mmol) D,L-Lactide, 4.03 g (34.7 mmol) glycolide, 0.028 g (0.07 mmol) Stannous octanoate and 0.0022 g (0.017 mmol) hydroxyethyl methacrylate (HEMA) were charged to a dry flask with an added condenser. The mixture was held under nitrogen and gradually heated to 150° C. The reaction mixture was held at 150° C. for two hours to produce a viscous amber liquid. This was diluted in THF, precipitated in methanol and dried under vacuum to isolate a PLGA polymer with 50/50 mole ratio composition, with methacrylate double bond end-group functionality (ref 174-63-1; 174 57-1).

### Example 13 C: EG-DSA-Initiated ROP of PLGA

PLGA copolymers such as those above and others can have poor compatibility or solubility in some oils or fragrances and so may benefit from the use of added diluents to facilitate the compatibilization of fragrance and polymer and the ensuing encapsulation. In addition to or in place of solubilizing diluent, which are small molecules, compatibilising polymers may be added (blended with), or incorporated into, the ring opening polymerized polymers. Since ring opening polymerization has a much more restricted choice of monomers to make a polymer there is limited opportunity to tailor the PLGA composition to be initially oil or fragrance compatible, PLGA's and similar polymers have poor compatibility or solubilization behaviors as a result of a limited choice of monomers for ROP. Thus, added diluents are added to aid initial solubilization or compatibility with fragrance. Adding high loadings of compatibilising diluents is not advised since they will dilute the cargo and/or potentially solubilize or plasticize the shell polymer if not removed, and evaporative removal processes are undesirable. However, we have discovered that by suitably designing modified polymers of PLGA a lower or even zero amount of diluent can be added. An example of this is the use of the hydroxyl groups on an oil- or fragrance-soluble prepolymer as initiator points for PLGA polymerization. An example is described below using a polyester based on dodecenylsuccinic acid or anhydride (DSA), and EG. The presence of the DSA based polyester, and similar molecules soluble in the chosen cargo, aids fragrance solubilization in the final polymer, even at its low levels as an initiator. Such a polymer, and other fragrance solubilizing polymers can also be blended with PLGAs, or other polymers that are not so soluble or compatible with fragrances or oils. This can help to reduce or remove the need for added, small molecule, diluents, the presence of high loadings of which may in some cases plasticize or weaken the shell wall formed. (Note reference to initiator or co-initiator in these aspects is referring to the ring opening polymerization applied to make the prepolymer or polymer—not to be confused with a radical initiator which may be used, as an option, in the oil-in-water encapsulation (shell formation from the prepolymer/polymer).

A DSA-EG (1:1 molar equivalent) prepolymer was synthesized: 2.8 g (45.1 mmol) EG was mixed with 10.02 g (37.5 mmol) DSA and charged to a Dean-Stark apparatus with 0.16 g pTSA (0.9 mmol) as catalyst. The mixture was heated to 190° C. under incrementally increasing vacuum for 24 hours to produce a polymer with an Mw of 3,640 gmol⁻¹. This polymer (ref 188-59), with its hydroxyl end groups was then used as initiator for ROP, as described immediately below.

DSA-EG(1 g, 0.000277 moles), Sn(Oct)2 (0.01 eq, 2.77×10−6 moles), glycolide (100 eq, 0.0277 moles), D,L-lactide (100 eq, 0.0277) were placed in a clean and dry round bottom flask equipped with a stirrer bar and then placed on the oil bath at 160° C. (external temperature) and stirred for 3 hours. A hard, white solid is formed. The resulting polymer molecular weight was Mw=24600 of gmol⁻¹ with a dispersity of 2.8 This polymer (ref 188-96-2/188-64) was soluble in warm (60-70° C.) fragrance, whereas the PLGA itself (ethylene glycol as initiator instead of DSA-EG) was not.

### Example 13D: ROP-PLGA with PCL Groups

5 g (34.7 mmol) D,L-Lactide, 4.03 g (34.7 mmol) glycolide, 7.91 g (69.3 mmol) caprolactone (CL), 0.0014 g (0.04 mmol) Stannous octanoate and 0.0011 g (0.017 mmol) hydroxyethyl methacrylate were charged to a dry flask with an added condenser. The mixture was held under nitrogen and gradually heated to 130° C. The reaction mixture was held at 130° C. for three hours to produce a viscous amber oil, with an Mw of 24,200 gmol⁻¹ and an Mw/Mn of 2.1 (ref 192-14-1).

### Example 14: Microencapsulation Using Prepolymers/Polymers Made by Ring Opened Polymerized (ROP) Polymers of Example 12

In these examples added diluents are used to facilitate encapsulation with PLGA and related polymers made by ring opening polymerization. While the prior also uses added solvents or diluent with PLGA and related polymers as described in the prior art, they use volatile organic solvents (e.g. DCM) which need to be removed by evaporative processes, unsuitable for many cargoes with volatile components). In our invention for this embodiment examples of added diluents are propylene carbonate, dimethyl carbonate, other alkylene carbonates triacetin, or oils such as Waglinol and various other esters, which can aid compatibilization of the fragrance with the polymer but which are not required to be removed to very low levels in many formulated end products—so can be tolerated or left in place. Other such diluents can be used including certain other esters, other carbonates, ethers, ketones, hydrocarbon oils, among others. Lower diluent addition levels could be accommodated with the use of DSA-EG PLGA and PLGA with PCL groups which aid solvation and can proceed with lower added solvent levels.

### Example 14A: ROP Capsules Made with EG-PLGA Functionalization

ENC-2026: EG-PLGA (4 g, 2.7 wt. %), propylene carbonate (16 g, 8.6 wt. %), Roc Green woody fragrance (30 g, 16.3 wt. %), Poval (12.79 wt. %) (6.6 g, wt. %), water (100 g, 54.2 wt. %), APS (ammonium persulfate) 0.32 g, wt. %). The EG-PLGA (4 g) polymer was solubilized in propylene carbonate (16 g) with stirring at 70-80° C. The polymeric solution becomes viscous and the fragrance (Roc Green woody fragrance (30 g)), was added to the flask and stirred for 30 min until complete solubilization (organic phase). An aqueous phase was prepared in a separate beaker in which water (100 g), and Poval (12.79 g) were stirred for 15 min. The organic phase is then poured into the aqueous phase and the mix stirred for 20 min. The latex is homogenized using IKA at 4200 rpm and placed in oil bath at 80° C. for 3 hours. Fragrance loading was found to be 6.4% in the slurry and 33.2% in the dried capsules (ref 188-56-1).

### Example 14B: ROP Capsules Made with HEMA Functionalization

ENC-2008: HEMA-PLGA (5 g, 2.7 wt. %), propylene carbonate (16 g, 8.6 wt. %), Roc Green woody fragrance (6 g, 10.6 wt. %), Poval (12.79 wt. %) (6.6 g, 1.56 wt. %), water (40 g, 71 wt. %), APS (ammonium persulfate) 0.32 g, wt. %).

HEMA-PLGA was solubilized in propylene carbonate at 70-80° C. The polymeric solution became viscous and the fragrance was added to the flask and stirred for 30 min until complete solubilization (organic phase). An aqueous phase was prepared in a separate beaker in which water, Poval and APS are stirred for 15 min. The organic phase is then poured in aqueous phase and stirred for 20 min. The latex is homogenized using IKA at 4200 rpm and placed in an oil bath at 80° C. for 3 hours, during which APS initiates reaction between methacrylate end-groups of the HEMA-PLA. Fragrance loading in the final reaction mixture was found to be 10% in the slurry and 29% on drying (ref 188-51-1).

### Example 14C: ROP Capsules Made with DSA-Polyester (Functionalization of PLGA with DSA-EG)—DSA-EG-PLGA Microcapsules

ENC-2034 premade polyester (DSA-EG; 3.3 g) was dissolved in 10.56 g propylene carbonate and stirred for 30 min at 80° C. Then, 19.8 g of ROC green fragrance woody is added, and the mixture is stirred for 15 min (organic phase). An aqueous phase composed by 52.1 g of water and 22.3 g of Poval at 11.8 wt. % in water was made up and stirred and added to the organic phase.

The emulsion is homogenized using an IKA mixer at 4200 rpm. The latex solution is place in an oil bath at 80° C. for 3 hours. As this is a linear polymer and no crosslinking this step is not compulsory and can be omitted or shortened, though it can be useful to ensure the fragrance is well dispersed and xanthan gum can be added if required. Fragrance loading in the slurry was measured to be 16.3%, an efficiency of 90% of what was added. Upon washing of the capsules with hexane (to remove any loosely bound, or free fragrance), a retained encapsulation efficiency of ˜87% was noted (ref 188-87-1).

3. In-Situ Emulsion/Mini-Emulsion Polycondensation Route from Monomeric Reactants or Precursors

In these examples of the invention a microcapsule with a lipophilic core and a biodegradable polymeric shell is demonstrated by: a) making an oil-in-water emulsion of an oil phase which comprises a di- or multifunctional-acid and a diol or multifunctional alcohol, a cargo, optionally with added diluent or solvent and/or aided by application of heat, and a water phase containing a stabilizer and/or other additives, b) adding a catalyst to one phase, c) forming the polymeric capsule shell wall by an in-situ oil-in-water polycondensation (esterification) polymerization reaction of the monomeric reactants or other precursors and d) obtaining the cargo encapsulated in a polymeric microcapsule shell.

The catalyst may be a sulfonic acid, a phosphoric acid, or other acid, or may be tin octanoate, tin hexanoate, stannic acid or a stannic acid derivative, tin oxide or a tin oxide derivative or other tin based compound, or is a lipase or other enzyme or compound able to catalyze esterification/condensation reactions.

The diluent will preferably be a water immiscible liquid at room temperature or readily meltable at moderate temperatures such as below 90° C. or less than 50° C. and may be a hydrocarbon oil, an alkane, a melted wax, an ester oil, a fatty acid ester, an aliphatic ester. Some specific examples include mineral oil, long chain alkanes such as hexadecane and the like, aliphatic esters such as esters of long chain acids such as caprylates, myristates, oleates, cocoates, palmitates, or stearates including isopropyl myristate as one example, or long chain esters of shorter chain acids or other monohydric or polyhydric esters.

We have discovered that certain uncrosslinked, biodegradable polyester microcapsules can surprisingly encapsulate an aggressive plasticizing or solvating lipophilic cargo such as a fragrance, another approach to achieve or demonstrate the same effect was discovered, wherein instead of making a polyester prepolymer for a subsequent oil-in-water encapsulation step (as variously described above) the polyester itself was made in-situ, concomitantly also forming a shell wall from its diol-diacid precursors, in the presence of the lipophilic cargo, via an in-situ mini-emulsion polycondensation, fragrance being used as an example cargo and a known challengingly aggressive example. A one-pot oil in water emulsion polymerization of acids and diols dissolved in fragrance was thus also demonstrated as a route to make capsules that can retain fragrance. Hydrophobic diacids and diols are preferred precursors for this process approach, though again with balance to have a structure that is non-persistent or biodegradable in the environment. A procedure for preparing polyester particles by mini emulsion polymerization is known. (Barre're, M. and Landfester, K. Polyester synthesis in aqueous miniemulsion. Polymer 44 (2003) 2833-2841), though such an approach has not been applied to making microcapsules, nor microcapsules of lipophilic cargoes, and not for making biodegradable microcapsules with lipophilic cargoes. Surprisingly an in-situ mini-emulsion (oil in water) polycondensation was able to be adapted to make microcapsules with an encapsulated lipophilic cargo (in this case a fragrance, but applicable to any oil soluble ingredient). Surprisingly this process approach was able to be achieved for such a plasticizing cargo, though with some important adaptations.

### Example 15: In-Situ Polycondensation Polymerization Process from Monomeric Reactants/Precursors

Into a 200 ml glass reactor vessel fitted with a 2 blade PET impeller stirrer powered by a mechanical stirrer at 400 rpm was placed 1,12-Dodecanedioic acid (3.3 g), 1,12-Dodecanediol (2.9 g) and Hexadecane (0.18 g) together with fragrance (2 g; ROC Green Woody from Robertet) and/or other oil, and heated to 95° C. to produce the oil phase. Deionised water (90 g, aqueous phase) was added to the oil phase and the reaction vessel was continued to be heated to 95° C. with stirring for 2 hours. Dodecylbenzene sulfonic acid (0.17 g) and deionized water (4.9 g) were then weighed into a 20 ml plastic beaker and stirred until homogeneous. This solution was then added to the reactor. The reaction was stirred for a further 3 hours at 95° C. The reactor was removed from the hot plate and stirring was continued until coming to room temperature. (Sample Ref: 151-45-1 when fragrance is excluded, for biodegradation testing samples).

A series of additional examples with other combinations of diols and/or diacids were also completed and variations in reaction conditions were also studied for example shorter stirring times, replacement of mechanical stirrer with a homogenizer (IKA (16000 rpm) or Silverson (4000 rpm for a few minutes).

Fragrance was shown to be encapsulated and capsules, which were typically in the range of 1-25 microns, were examined under an optical microscope and shown to emit a cargo when pressure was applied to them. Some of the cargo in some cases was encapsulated more strongly retained (which is shown in the tabulated data below, typically after hexane solvent washing of the capsules to remove free or loosely bound fragrance)

TABLE 6
Fragrance loading with mini-emulsion polymerization of diacids and diols

| | | Fragrance Loading |
|---|---|---|
| | | (%) in isolated |
| Composition (Diol/Diacid) | Reference Number | capsules/particles |
| C12 diol/C12 diacid with 50/50 waglinol/fragrance - stirring | 151-48-1/ENC 2014 | 12.2 |
| C12 diol/C12 diacid with fragrance only - stirring | 151-48-2/ENC 2015 | 29.5 |
| C12 diol/C12 diacid with 50/50 waglinol/fragrance - homogenizer | 151-48-3/ENC 2016 | 13.6 |
| C12 diol/C12 diacid with fragrance only - homogenizer | 151-48-4/ENC 2017 | 23.6 |
| C12 diol/C6 diacid with stirring fragrance only | 151-46-3/ENC 1880 | 8.9 |
| Succinic acid with 1,12 dodecanediol & fragrance only | 151-46-9/ENC 1932 | 9.3 |
| 1,12 dodecanoic acid with 1,6-Hexanediol and fragrance only | 151-47-3/ENC 1937 | 10.3 |
| 1,12 dodecanoic acid with 1,8-Octanediol and fragrance only | 151-47-4/ENC 1938 | 9.9 |
| 1,12 dodecanoic acid with 1,10-Decanediol and fragrance only | 151-47-5/ENC 1939 | 10.8 |

Branched or crosslinked structures can be produced using multifunctional acids and/or multifunctional polyols. Emulsion stability and rate of reaction may be improved in some cases by replacing the dodecylbenzene sulfonic acid, which acts as both a catalyst and a surfactant with additives which are specifically optimized to fulfil each role, for example Fast Cat 4100 or p-TSA and alternative surfactants.

For example, into a 200 ml glass reactor vessel fitted with a 2 blade PET impeller stirrer powered by a mechanical stirrer at 400 rpm was placed 1,12-Dodecanedioic acid (3.3 g, 144 mmol), castor oil, a lipophilic triol (8.96 g, 96 mmol)g) and Hexadecane (0.18 g) together with fragrance (2 g; ROC Green Woody from Robertet) and/or other oil, and heated to 95° C. to produce the oil phase. Deionised water (90 g, aqueous phase) was added to the oil phase and the reaction vessel was continued to be heated to 95° C. with stirring for 2 hours. Dodecylbenzene sulfonic acid (0.17 g) and deionized water (4.9 g) were then weighed into a 20 ml plastic beaker and stirred until homogeneous. This solution was then added to the reactor. The reaction was stirred for a further 3 hours at 95° C. The reactor was removed from the hot plate and stirring was continued until coming to room temperature. Microcapsules in a slurry were formed with fragrance inside.

The solids of this reaction mixture can be increased to attain even higher fragrance loadings. For example, into a 200 ml glass reactor vessel fitted with a 2 blade PET impeller stirrer powered by a mechanical stirrer at 400 rpm was placed 1,12-Dodecanedioic acid (3.3 g, 144 mmol), castor oil, a lipophilic triol (8.96 g, 96 mmol)g) and Hexadecane (0.18 g) together with fragrance (30 g; ROC Green Woody from Robertet) and/or other oil, and heated to 95° C. to produce the oil phase. Deionized water (60 g, aqueous phase) was added to the oil phase and the reaction vessel was continued to be heated to 95° C. with stirring for 2 hours. Dodecylbenzene sulfonic acid (0.17 g) and deionized water (4.9 g) were then weighed into a 20 ml plastic beaker and stirred until homogeneous. This solution was then added to the reactor. The reaction was stirred for a further 3 hours at 95° C. The reactor was removed from the hot plate and stirring was continued until coming to room temperature. Capsules in a slurry were formed with fragrance inside.

### Example 16: Biodegradation Testing of Polymer Shells Made by In-Situ Mini-Emulsion Polycondensation

Analogous blank polyester particles (for example our REF 151-45-1 and other compositions in Table 5) were synthesized as described above (example 12) but without the addition of fragrance to make equivalent particles with no cargo (could compromise or complicate biodegradation testing) for biodegradability testing. For example, in OECD 306 testing conditions (in seawater, one of the least aggressive of aquatic media options for biodegradation testing) a biodegradation of 21% after 28 days was measured for a sample of the polymer based on dodecanedioic acid dodecanol (C12 diacid-C12 diol), suggesting it could be classified as inherently biodegradable. This particular sample was among the more hydrophobic of the examples made (note as per this test standard, sodium benzoate was used as a reference and measured 70% biodegradation after 28 days). Higher degradation levels would likely be achieved with either longer times and/or different OECD test methods such as OECD 302 or 301 in other aquatic media such as activated sludge. Similarly, higher % biodegradation would be reasonably expected for the less hydrophobic polyester shell structures made. As previously mentioned, the seawater test (OECD 306, as used here) is generally considered to be a mild medium for biodegradation testing and certainly less aggressive as a medium for biodegradation testing, compared to other media mentioned in those other tests (activated sludge; surface water).

4. Crosslinked Poly β-Amino-Ester and/or Polyβ-Thio-Eester Route by In-Situ Oil-In-Water Addition Polymerization

In these examples of the invention a microcapsule with a lipophilic core and a biodegradable polymeric shell is demonstrated by: (a) making an oil-in-water emulsion of an oil phase which comprises all donor and acceptor reactants and a cargo, optionally with added diluent or solvent and/or aided by application of heat, and a water phase containing a stabilizer or emulsifier, optionally with other additives, (b) optionally adding a catalyst or initiator to one phase (c) forming the polymeric capsule shell wall by an in-situ oil-in-water addition polymerization reaction of the donor and acceptor reactants; and (d) obtaining the cargo encapsulated in a polymeric microcapsule shell.

The diluent will preferably be a water immiscible liquid at room temperature or readily meltable at moderate temperatures such as below 90° C. or less than 50° C. and may be a hydrocarbon oil, an alkane, a melted wax, an ester oil, a fatty acid ester, an aliphatic ester, or an alkylene carbonate. Some specific examples include mineral oil, long chain alkanes such as hexadecane and the like, aliphatic esters such as esters of long chain acids such as caprylates, myristates, oleates, cocoates, palmitates, or stearates including isopropyl myristate as one example, or long chain esters of shorter chain acids or other monohydric or polyhydric esters.

The catalyst is preferably a base added to the oil phase, such as triethylamine or another tertiary amine.

### Example 17 Ref 210-26-1: Michael Addition Polymerization-Encapsulation n Using of a Penta/Hexa-Acrylate(1 Eq) and a Tetrathiol (0.9 Eq), with a Diamine Also Present TMPP (0.1 Eq) (Ratios Shown are Molar Equivalents of the Reactive Groups: Respectively: Acrylate, —SH; —NH)

0.61 g (3 mmol) 4,4′ trimethylenedipiperidine (TMPP) and 6.28 g (13 mmol) pentaerythritol tetrakis(3-mercaptopropionate) (PTTKMP, tetrathiol) was added to a glass jar with 0.36 g (3.6 mmol) triethylamine. To this jar a magnetic stirrer bar was added along with 53 g of fragrance, R1439-13 Sunburst Fresh (Robertet, FR). The mixture was stirred until both monomers had dissolved. To this mixture, 11 g propylene glycol dicaprate/caprate (Waglinol 2/7680) was added to form an oil phase. In parallel, an aqueous phase of 206 g water, 32.8 g of a 10% w/w solution of polyvinyl alcohol and 0.2 g Agitan 295 defoamer was added and stirred at 150 rpm using an overhead stirrer. A syringe was charged with 5 g (10 mmol) dipentaerythritol penta/hexaacrylate (DiPETA penta/hexa acrylate) and this was added to the oil phase, stirring for 30 s until the acrylate monomer was well mixed. The oil phase was quickly added to the aqueous phase and allowed to stir for 5 minutes to form a coarse emulsion. This emulsion was homogenised with a IKA Magic LAB at 4000 rpm and charged into a 1 L resin reaction pot with an anchor stirrer. The reaction mixture was stirred at 150 rpm and 35 C for 24 hours and a slurry of solid microcapsules containing about 17 wt % (in whole slurry) fragrance inside was obtained.

### Example 18 Ref 210-86-1: Michael Addition Polymerization—Encapsulation with a Triacrylate (1.0), a Tetra Thiol (0.9) and a Cyclic Secondary Diamine, TMPP (0.1)

0.58 g (2.7 mmol) 4,4′trimethylenedipiperidine and 6.00 g (12.3 mmol) pentaerythritol tetrakis(3-mercaptopropionate; tetrathiol) was added to a glass jar with 0.36 g (3.6 mmol) triethylamine. To this jar a magnetic stirrer bar was added along with 53 g of fragrance, R1439-13 Sunburst Fresh (Robertet, FR). The mixture was stirred until both monomers had dissolved. To this mixture, 11 g propylene glycol dicaprate/monocaprate (Waglinol 2/7680) was added to form an oil phase. In parallel, an aqueous phase of 206 g water, 32.8 g of a 10% w/w solution of polyvinyl alcohol and 0.2 g Agitan 295 defoamer was added and stirred at 150 rpm using an overhead stirrer. A syringe was charged with 5.43 g (18.2 mmol) pentaerythritol triacrylate and this was added to the oil phase, stirring for 30 s until the acrylate monomer was well mixed. The oil phase was quickly added to the aqueous phase and allowed to stir for 5 minutes to form a coarse emulsion. This emulsion was homogenized with a IKA Magic LAB at 4000 rpm, and charged into a 1 L resin pot with an anchor stirrer. The reaction mixture was stirred at 150 rpm and 35 C for 24 hours and a slurry of solid microcapsules containing about 17 wt % (in slurry) fragrance inside was obtained.

The same general procedure as described above was applied or able to be repeated to successfully make microcapsules with fragrance as a core using other reactants such as listed below. Note here and throughout the following examples (all ratios are molar equivalents of the functional reactive groups (acrylate., N—H, S—H).
215-45-1: TMPTA (1.0, trimethylolpropane triacrylate)-TMPP (0.1)—PTHKMP hexa thiol (0.9, pentaerythritol hexakis (3-mercaptopropionate).
215-49-1: TMPTA (1.0, trimethylolpropane triacrylate)—PTHKMP hexa thiol (1.0, pentaerythritol hexakis (3-mercaptopropionate). (Makes β-thio ester only: no amine).
215-46-1: PETA-tri (1.0, pentaerythritol triacrylate)-TMPP (0.1)—PTHKMP hexa thiol (0.9, pentaerythritol hexakis (3-mercaptopropionate).
215-50-1: PETA-tri (1.0, pentaerythritol triacrylate)—PTHKMP hexa thiol (1.0, pentaerythritol hexakis (3-mercaptopropionate). (Makes β-thio ester only: no amine).
215-55-1: DiPETA (1.0, DiPentaerythritol triacrylate)-TMPP (0.1)—tri-thiol (0.9, (trimethylol propane tris(3-mercaptopropionate).
215-56-1: DiPETA (1.0, DiPentaerythritol triacrylate)—tri-thiol (1.0, (trimethylolpropane tris(3-mercaptopropionate). (Makes β-thio ester only: no amine).

### Example 19 Ref No: 210-91-1—Michael Addition Polymerization—Encapsulation with a Triacrylate (1.0)—a Tetra Thiol (0.9) and TMPP (0.1) with Added Radical Initiator (APS/TMEDA)

0.58 g (2.7 mmol) 4,4′trimethylenedipiperidine (TMPP) and 6.00 g (12.3 mmol) pentaerythritol tetrakis(3-mercaptopropionate) (tetrathiol) was added to a glass jar with 0.36 g (3.6 mmol) triethylamine. To this jar a magnetic stirrer bar was added along with 53 g of fragrance, R1439-13 Sunburst Fresh (Robertet, FR). The mixture was stirred until both monomers had dissolved. To this mixture, 11 g propylene glycol dicaprate/caprate (Waglinol 2/7680) was added to form an oil phase. In parallel, an aqueous phase of 206 g (11.44 mol) water, 32.8 g of a 10% w/w solution of polyvinyl alcohol and 0.2 g Agitan 295 defoamer was added and stirred at 150 rpm using an overhead stirrer. A syringe was charged with 5.43 g (18.2 mmol) pentaerythritol triacrylate and this was added to the oil phase, stirring for 30 s until the acrylate monomer was well mixed. The oil phase was quickly added to the aqueous phase and allowed to stir for 5 minutes to form a coarse emulsion. This emulsion was homogenised with a IKA Magic LAB at 4000 rpm and charged into a 1 L resin pot with an anchor stirrer. The reaction mixture was stirred at 150 rpm. To this, 0.71 g (3.1 mmol) of ammonium persulfate, 25% w/w in water was added. The mixture was degassed with a nitrogen sparge for 30 minutes. The mixture was held under a nitrogen blanket, and a shot of 0.5 g (4.3 mmol) tetramethyl ethylenediamine, 25 w/w in water was added. The temperature was increased to 35° C. and the nitrogen blanket was maintained for 6 hours. Following this, the nitrogen blanket was removed, and the reaction mixture was allowed to stir at 35° C. for a further 18 hours and a slurry of solid microcapsules containing about 17 wt % (on slurry) fragrance inside was obtained.

### Example 20: 201-82-1—Michael Addition Polymerization—Encapsulation with an Amine Only Donor with a Triacrylate (1.0)-TMPP (1.0)—and No Added Additional Catalyst (Self-Catalyzed by Amine)

2.80 g (13.7 mmol) 4,4′trimethylenedipiperidine (TMPP) was added to a glass jar. To this jar a magnetic stirrer bar was added along with 53 g of fragrance, R1439-13 Sunburst Fresh (Robertet, FR). The mixture was stirred until both monomers had dissolved. To this mixture, 11 g propylene glycol dicaprate/monocaprate (Waglinol 2/7680) was added to form an oil phase. In parallel, an aqueous phase of 206 g (11.44 mol) water, 32.8 g of a 10% w/w solution of polyvinyl alcohol and 0.2 g Agitan 295 defoamer was added and stirred at 150 rpm using an overhead stirrer. A syringe was charged with 2.64 g (8.9 mmol) pentaerythritol triacrylate and this was added to the oil phase, stirring for 30 s until the acrylate monomer was well mixed. The oil phase was quickly added to the aqueous phase and allowed to stir for 5 minutes to form a coarse emulsion. This emulsion was homogenized with a IKA Magic LAB at 4000 rpm and charged into a 1 L resin pot with an anchor stirrer. The reaction mixture was stirred at 150 rpm and 35 C for 24 hours and a slurry of solid microcapsules containing fragrance inside was obtained.

Variations to this process for making the fragrance capsules included examples of adding minor amounts (0.5 wt % or less and preferably 0.2 wt % or 0.1 wt % or less) of either a reactive monomeric acrylate such as β-carboxyethyl acrylate or hydroxyethyl acrylate or methacrylate at the start, part way through the reaction or towards the end to capture any residual thiols, and/or radical initiators such as APS/TMEDA (at or soon after reaction start, part way through or towards the end) to mop up any residual acrylate and/or thiol or with addition of chitosan or other insoluble polymer powder.

### Example 21: In-Situ Michael Addition Polymerization—Encapsulation with Added Polyester Prepolymer with Reactive Unsaturation (Ref 210-54-1)

A polymer with itaconate double bonds synthesized as described above was used as a component in the Michael Addition polymerization-encapsulation route. The polymer in this example was a polyester Ref 201-43-1 SA (0.75)/IA (0.25)/1,12 DDD (succinic acid/itaconic acid and 1,12 dodecanediol, premade using FASCAT 4100 as a catalyst for the polyester forming reaction (160° C./24 hrs). A reaction flask was prepared in a water bath set at 35° C. The premade itaconate containing polyester prepolymer (0.83 g; made from succinic acid (37.5 mol %) itaconic acid (12.5 mol % and dodecanediol (50 mol %)) was dissolved in 53 g fragrance (R1439-13, woody green) in a 150 ml beaker with stirring to which was added a multifunctional acrylate (DiPEHA, dipentaerythritol penta/hexa acrylate, 3.90 g). The aqueous phase (external phase) was prepared in parallel in another beaker (500 ml) comprising water (206 g), Agitan 295 (0.2 g) and polyvinyl alcohol (Poval 40-88, 32.8 g of a 10% solution).

A mixture of the thiol (PTHKMP, pentaerythritol hexakis (3-mercaptopropionate, 7.17 g), catalyst (triethylamine, 0.36 g) and diluent oil (Waginol, 11.0 g) was made up in another beaker and added to the acrylate-polymer, with stirring for about 30 seconds to form the internal phase with all reactants present.

The aqueous (external phase) was then added soon after addition of the thiol mixture and the whole (both phases) homogenized (IKA homogenizer) at 4000 rpm and then immediately transferred into the reaction flask and reacted at 35° C. with stirring for about 24 hours. A slurry of microcapsules with ˜17 wt % (total slurry) fragrance inside was produced.

### Example 22: Pre-Reacting Amine Donors First in a Michael Addition Polymerization—Encapsulation

In some circumstances it has been found that amines can be more stably or readily incorporated into a hybrid poly-β-amino ester co-β-thio ester polymer shell by firstly reacting or capping the amine groups with acceptor molecules, as a first step, in bulk or with added fragrance and/or diluent carrier, via a pre-reaction with all or a portion of the acceptor (e.g. acrylate), optionally aided by heating. This is particularly useful in the case where the amine is more water soluble and so not well suited to an in-situ oil in water polymerization wherein all reactants are to be in the oil phase. Such pre-end-capping of amine donors results in substantial or near whole transformation of N—H groups to make adducts or oligomeric derivatives, prepolymers of the amine with the multifunctional acrylate or other acceptor, and so all or most of the amines now bear acrylate bonds (where NH bonds were previously), rendering them relatively more lipophilic or less hydrophilic compared to the starting amine itself. Such adducts or initial prepolymer or oligomeric products remain as relatively low molecular weight adducts which may remain soluble in cargo and/or added diluent and/or added excess acrylate, or which can be otherwise readily solubilized, optionally aided by heat. (Note: This is also a source of prepolymers for route 1 described earlier and the same general procedures can be used to make poly β-amino ester or poly β-thio ester prepolymers (or their copolymers) for use in the other routes described using a prepolymer. They can bear acrylate (reactive unsaturation) for use in prepolymer routes). The encapsulation stage of this pre-reaction route variant is progressed with homogenisation/dispersion and added further donor (thiol donor) at a stoichiometrical equivalence (or approximately so) in terms of available acrylate groups remaining after end-capping of the amine donor).

### Example 22A: Microcapsule Reference: 215-32 (Molar Equivalent Ratios: PTHKMP (0.8)/TMPP (0.2)/PETA (Tetra; (1.0))—Sequential Reaction

A difunctional amine donor TMPP (1.18 g) was dissolved in about half of the fragrance (Green Woody). A multifunctional acrylate, pentaerythritol tetraacrylate (4.94 g) was added and the mixture stirred for 2 hours in a beaker to allow substantial end-capping of amine NH's with acrylate. In parallel, an external (water) phase was prepared that comprised (in a 500 ml beaker): water (205.9 g), Poval 40-88 (32.81 g of a 10% solution), and Agitan 295 (0.20 g). The rest of the internal (oil) phase was pre-mixed in another beaker: (hexathiol, pentaerythritol hexakis (3-mercaptopropionate PTHKMP, 5.85 g), Waglinol (10.68 g), the remainder of the fragrance (such that the total fragrance used was 54.32 g) and triethylamine as catalyst (0.36 g). This was then mixed (˜30 seconds) with the acrylate-TMPP fragrance mixture to make a complete oil phase.

This oil phase was added to the water phase in the 500 ml beaker and stirred for a ˜1 minute and the whole mixture was then homogenized with an Ika homogenizer for ˜1 minute at 4000 rpm—and then transferred to a reaction flask and reacted with stirring for 24 hours. A slurry of microcapsules with fragrance inside was formed.

### Example 22B: Microcapsule Reference 215-33: PTHKMP (0.9)/TMPP (0.1)/PETA (Tetra)(1.0)—Sequential Reaction

The same procedure as above (Ex 22A) was applied for a reaction with TMPP at half the (molar equivalent) compared to 215-32 above, generating a slurry of microcapsules (ref 215-33) with fragrance inside. Details were: Internal Phase: PETA tetraacrylate (5.06 g), TMPP, (2.41 g)—pre-reacted as above in half of the fragrance; PTHKMP (4.49 g), R1439-13 Green Woody (53.42 g), Waglinol (10.68 g), and Triethylamine (0.36 g). External Phase: Water (205.90 g), Poval 40-88 (10% sol; 32.81 g), Agitan 295 (0.20 g).

Further examples following the same procedure as above were performed successfully to make microcapsules with a fragrance as a core, using the reactants as below;
215-47-1: TMPTA (1.0, trimethylolpropane triacrylate)-TMPP (0.1)—PTHKMP hexa thiol (0.9, pentaerythritol hexakis (3-mercaptopropionate).
215-48-1: PETA-tri (1.0, pentaerythritol triacrylate)-TMPP (0.1)—PTHKMP hexa thiol (0.9, pentaerythritol hexakis (3-mercaptopropionate).
215-57-1: DiPETA (1.0, Di pentaerythritol triacrylate)-TMPP (0.1)—tri-thiol (0.9, (trimethylol propane tris(3-mercaptopropionate).

### Example 22 C: Further Examples of Sequential Pre-Reacting of Amines or Water-Soluble Donors Prior to Encapsulation with a Second Donor

Using the same procedure of Example 22A for a sequential pre-reaction, other microcapsules were able to be made via the in-situ polymerization—encapsulation route using water soluble or sensitive donors.

Details of microcapsule ref 215-41: Internal Phase: DiPETA Penta/hexaacrylate: 5.40 g; HMDA: 0.33 g (pre-reacted); PTTKMP: 6.23 g; R1439-13 Green Woody: 53.42 g; Waglinol: 10.68 g; Triethylamine: 0.36 g. External Phase: Water (205.90 g); Poval 40-88 (10% sol; 32.81 g), Agitan 295 (0.20 g).

Examples 22 D: The same procedure as above of pre-reacting an amine donor with an acrylate acceptor, and then reacting with a thiol donor in an oil in water emulsion polymerization to form microcapsules, was applied to other diamines such as isophorone diamine (IPDA), dodecane diamine, and hexamethylene diamine (HMDA), and using multifunctional thiols. Such other diamines demonstrate the breadth of this approach (some are particularly water soluble primary diamines, rendered less water soluble or insoluble by the pre-reaction stage) and were used at stoichiometries (approx. 1:1 in total donor H's to total acceptor groups) targeting a reactive functionality (f) of 4 (NH), and also in other examples a reactive functionality (f) of 2 (NH), so a lower acceptor level (slight excess of donor), likely to lead to some residual NH in the formed polymeric shells of the latter (f2) ratio. The thiol used in these examples was a hexa-thiol, and the acrylate a tetra-acrylate. All combinations formed capsules with ˜17 wt % fragrance (on slurry) encapsulated and with visible release of fragrance when crushed under a microscope slide.

TABLE 7
Further examples of Amine-Thiol compositions used for
Microencapsulation via pre-reaction of an amine donor

| Capsule | | |
|---|---|---|
| Reference | Donors | Acceptor |
| 215-41-1 | HMDA (0.1; @ f2NH, or 0.05) PTHKMP (0.9; f4) | PETA tetra acrylate (1.0; f4) |
| 215-42-1 | HMDA (0.1; f4) PTHKMP (0.9; f6) | PETA tetra acrylate (1.0; f4) |
| 215-51-1 | IPDA (0.1; @ f2 NH, or 0.05) PTHKMP (0.9; f6) | PETA tetra acrylate (1.0; f4) |
| 215-52-1 | IPDA (0.1; f4) PTHKMP (0.9; f6) | PETA tetra acrylate (1.0; f4) |
| 215-59-1 | DDDA (0.1 f4) PTKHP (0.9 f6) | PETA tetra acrylate (1.0; f4) |

Notes:
PTTKMP: pentaerythritol tetrakis (3-mercaptopropionate)
PTHKMP: pentaerythritol hexakis (3-mercaptopropionate)
IPDA: isophorone diamine
HMDA: hexamethylene diamine
DDDA: 1,12 dodecanediamine
DiPETA: DiPentaerythritol triacrylate (hexa/penta functional)
PETA tetra: pentaerythritol tetra acrylate

### Example 22 E

The method of above in Examples 22 was also applied to a microcapsule with a 10 mol % excess of acrylate functionality (10 mol eq % excess). Microcapsules (17 wt % fragrance in whole slurry) were successfully made which clearly released fragrance upon crushing. The capsule was (ref 210-62-1) made from a pre-reaction of a difunctional secondary amine, TMPP (0.1 mole eq), with a tetrafunctional acrylate (pentaerythritol tetra-acrylate (PETA-tetra, 1.1 mol eq), then subsequently mixed with a hexa functional thiol (DiPentaerythritol Hexakis(3-mercaptopropionate, (DiPTHKMP), 0.9 mol eq) and homogenized with the water phase then reacted via an in-situ oil in water emulsion polymerization to form the capsule shell.

### Example 22 F

The method of above in Examples 22 was also applied to a microcapsule made without an additional catalyst but using the amine donor as a self-catalyst. Microcapsules (17 wt % on slurry) were successfully made which clearly released fragrance upon crushing. The capsule (ref 210-61-1) was made from a pre-reaction of a difunctional secondary amine, TMPP (0.1 mole eq), with a tetrafunctional acrylate (pentaerythritol tetra-acrylate; PETA-tetra, 1.0 mol eq), then subsequently mixed with a hexa functional thiol (DiPentaerythritol Hexakis(3-mercaptopropionate, (DiPTHKMP), 0.9 mol eq) and homogenized with the water phase and then reacted via an in-situ oil in water emulsion polymerization to form the capsule shell.

### Example 22 G

The method of above in Examples 22 was also applied to a microcapsule without the use of an added diluent in the process. Additional fragrance (˜11 g) was used in place of the Waglinol of the Examples 22 above. Microcapsules (˜20 wt % fragrance on slurry) were successfully made which clearly released fragrance upon crushing. The capsule was (ref 210-63-1) made from a pre-reaction of a difunctional secondary amine, TMPP (0.1 mole eq), with a tetrafunctional acrylate (pentaerythritol tetra-acrylate (PETA-tetra, 1.0 mol eq), then subsequently mixed with a hexa functional thiol (DiPentaerythritol Hexakis(3-mercaptopropionate, (DiPTHKMP), 0.9 mol eq) and homogenized with the water phase, then reacted via an in-situ oil in water emulsion polymerization to form the capsule shell.

### Example 23: Further Examples of the Michael Additional In-Situ Polymerization—Encapsulation Process—Here with Tetra Functional or Higher Functionality Donors and Acceptors

Various samples using various other combinations of either a tetra-functional thiol or a hexa-functional thiol (with or without added TMPP as additional donor) with either a tetrafunctional acrylate or a penta/hexa acrylate were prepared using the same procedures as above. See Tables below. All were microcapsules were successfully made with fragrance as cargo following the method above and all formed capsules and with ˜17 wt % fragrance inside (˜90-100% encapsulation efficiency). All exhibited a fragrance release on crushing and also showed a fragrance bloom after formulation into a representative fabric conditioner formulation (pH3). See example data further below.

Equivalent weights were largely matched so that the overall stoichiometry of donor(s) (thiols with or without TMPP or other amine) were approximately equivalent to that of the acceptors (acrylates in these examples). Where added TMPP was present it was preferentially present at about 10 or 20 eq. mol %. the remaining donor being a multifunctional thiol, and more preferably 10 mol % (the thiol mol % subsequently adjusted to ensure overall matching of stoichiometries of donors to acceptor (so moles of NH and SH groups-together) were equivalent to the moles of acrylates groups present).

All were stable ‘as made’ and some were stable when tested at 40° C. neutral and some at pHs away from neutral (capsules still clearly ‘in-tact’ and also showing release on crushing) after 40C aging (14 days) in pH's 3, 7 and 11 and ‘as made’ in their slurries. It was noted some with higher water soluble or sensitive amine equivalent contents more than about 20 mole % (based on equivalent groups of donors) were softer or less retentive with the use of the aggressive plasticizing fragrance cargo used here but such capsules would be expected retain other lipophilic cargoes successfully, and show more rapid biodegradation profiles compared to analogous lower amine content capsules.

TABLE 8
A summary of some example fragrance microcapsules some of which were sensory
panel tested for fragrance bloom (see examples of results further below)

| | Acrylate | Thiol | Amine |
|---|---|---|---|
| | (functionality; | (functionality; | (functionality; |
| Capsule with fragrance - ref | mol eq C═C) | mol eq SH) | mol eq NH) |
| 210-26-1; 215-01-1 | Penta/Hexa (5/6; 1.0) | Tetra (4; 0.9) | TMPP (2; 0.1) |
| 215-15-1 | Tetra (4; 1.0) | Hexa (6; 0.9) | TMPP (2; 0.1) |
| 215-09-1 | Penta/Hexa (5/6; 1.0) | Hexa (6; 0.9) | TMPP (2; 0.1) |
| 215-38-1 | Penta/Hexa (5/6; 1.0) | Tetra (4; 0.9) | TMPP (2; 0.1) |
| 215-24-1 | Penta/Hexa (5/6; 1.0) | Hexa (6; 1.0) | — |
| 215-16-1 | Tetra (4; 1.0) | Hexa (6; 1.0) | — |
| 215-12-1 | Penta/Hexa (5/6; 1.0) | Tetra (4; 1.0) | — |
| 215-32 -1 | \|Tetra (4; 1.0) | Hexa (6; 0.8) | TMPP (2; 0.2) |
| 215-35-1 | Tetra (4; 1.0) | Hexa (6; 0.9) | TMPP (2, 0.1) |
| 201-87-1; 201-87-3: 87-1 with added | Tetra (4; 1.0) | Tetra (6; 0.9) | TMPP (2; 0.1) |
| xanthan gum) both 17% fragrance | | | |
| encapsulated) | | | |
| 201-86-1 | Tri (3; 0.9) | Tetra (0.9) | TMPP (2; 0.1) |
| 201-90/91/92: Same as 201-86-1 but: | Tri (3; 0.9) | Tetra (0.9) | TMPP (2; 0.1) |
| 201-90: with APS/TMEDA (start) | | | |
| 201-91: with APS/MEDA (end) | | | |
| 201-92: with added chitosan | | | |
| 201-29-1 = 210-26-1 with | Penta/Hexa (5/6; 1.0) | Tetra (4; 0.9) | TMPP (2; 0.1) |
| APS/TMEDA (start) | | | |
| 201-32-1 = 210-26-1 with 0.1% | Penta/Hexa (5/6; 1.0) | Tetra (4; 0.9) | TMPP (2; 0.1) |
| xanthan gum (start) | | | |
| 212-04-1 = 210-26-1 with slight | Penta/hexa (5/6 1.1) | Tetra (4; 0.9) | TMPP (2; 0.1) |
| excess acrylate | | | |

Notes:
Tetra thiol = pentaerythritol tetrakis (3-mercaptopropionate (PTTKMP)
Hexa thiol = Pentaerythritol hexakis (3-mercaptopropionate) (PTHKMP)
Penta/hexa acrylate = dipentaerythritol penta/hexaacrylate (DiPEHA)
Tetra acrylate = Pentaerythritol tetraacrylate
Tri acrylate = Pentaerythritol triacrylate
TMPP = 4,4′trimethylenedipiperidine
TMPTA = trimethylolpropanetriacrylate

### Example 24 (Ref 201-60-1): Comparative Example of Prior Art—Using Interfacial Polymerization and a Water Soluble Di-Thiol as Sole Donor

An example from the publication, Liao et al, “Fragrance-containing microcapsules based interfacial thiol-ene polymerization”. (J. Appl. Polym. Sci. 2016, doi: 10.1002/App.43905) was completed (replicated)—using the pentaerythritol tetra-acrylate and the prescribed water soluble dithiol, dithiothreitol, which was added in significant excess via the aqueous phase, according to the procedure published. While capsule was formed with the fragrance used here (Green Woody) they were very soft and not robust, appearing to be plasticised by the fragrance used and, the product slurry had a very significant and overpoweringly unpleasant thiol odour which masked any presence of fragrance. These points were suggested earlier in the prior art discussion and show that the use of a water soluble dithiol donor in an interfacial polymerization process, and used in significant excess for that process, do not form useful fragrance retaining capsules.

For the prior art reference example of using classical interfacial polymerization and water soluble dithiol in molar equivalent excess. trimethylolpropane triacrylate (TMPTA; sample A1 in the paper) was used since it is described as having the best combination of high efficiency and stability. The amounts used were:
Oil Phase: TMPTA (0.2615 g; A1); R14-3913 fragrance (15 g);
Aqueous Phase: Mowiol 40-88 (2%; 45 g); and
Feed mixture (dissolved in water): Dithiothreitol (0.5885 g; D1); K₂CO₃ (0.04 g); Water (12 g).

The aqueous, oil and feed phases described above were each separately prepared. The oil and aqueous phase were homogenized (10000 rpm for 3 mins using an Ultra-turrax 25). The homogenized emulsion was transferred to a reactor and stirred. The feed mixture was added dropwise with stirring. The reaction mixture was stirred for 3 hours. A slurry of microcapsules was formed. A very strong unpleasant thiol odour was noted and the capsules became soft on standing.

### Example 25: Spray Dried Microcapsules with Fragrance Inside, Prepared Michael Addition Polymerization-Encapsulation (Ref: 210-36-1, 210-48-1)

Microcapsules (210-36-1) containing ˜17 wt % green wood fragrance were made following a similar procedure, using a penta/hexa-acrylate (1 mol eq), a tetra thiol (0.9 mol eq) and TMPP (0.1 mol eq; pre-reacted). Details are as below:

Internal Phase: All reactants: DiPEHA (penta/hexaacrylate) (functionality 5.5; 5.29 g) trimethylene dipiperidine (TMPP, functionality 2, 0.58 g) and pentaerythritol tetrakis(3-mercaptopropionate) (PTTKMP, functionality 4, 6.10 g) with R1439-13 Green Woody, Waglinol (53 g), and Triethylamine (catalyst; 0.36 g).

External Phase: Water (206 g), Poval 40-88 (10%. sol, 32.8 g), Agitan 295 (0.2 g).

Aqueous phase components were mixed: Water and Poval, stirred; Agitan added. Oil phase components were mixed in two beakers: DiPEHA, TMPP+23 g fragrance in beaker 1. PTTKMP, TEA, waglinol/fragrance in beaker 2. Both beakers were stirred for 1 hr. Beaker 1 mixture was added to beaker 2 and stirred for 30 s. Then the aqueous phase was added and the whole mixed for 5 mins with homogenization (Ika) at 4000 rpm. Xanthan gum was added (0.1% of total slurry) and the mixture was transferred to a reaction flask and reacted for 24 hrs at 35° C. with stirring.

Spray Drying Method—for 210-48-1 (Using a Buchi B290 spray drier): 277 g of Slurry @ 25% Solids was diluted to 5% solids with 1385 g with Deionized water, 8.31 g (3% of 210-36-1 slurry) of Sipernat 50S silica was added and mix thoroughly with an overhead stirrer for 30 mins. This was then charged over time to produce a spray dried sample 210-48-1. Conditions were: Inlet temperature: 190° C.; Air Flow 4 cm (10 L/min); Feed setting 18.

The free flowing dried capsules isolated could be redispersed in water stably and were formulated into a representative fabric conditioner system and showed a bloom or fragrance release upon testing.

### Example 26: Microcapsules with Peppermint Oil (Ref 210-53-1)

The microcapsules produced by the invention can contain many lipophilic or oil solubilized cargoes. The ready successful encapsulation of dichloromethane as a substantially water immiscible solvent (used for biodegradation testing samples) demonstrates such versatility. Fragrance was used in most examples since it is considered an aggressively plasticizing (difficult) cargo to encapsulate and retain. Here an example with another oil based cargo is described, using a similar procedure as above.

A sealed reactor flask was prepared in a water bath at 35° C.

TMPP (0.58 g) was dissolved in 33 g of peppermint oil in a 250 ml beaker. When dissolved the rest of the oil (internal) phase (6.50 g hexathiol, PTHKMP; 11 g Waglinol; 0.36 g triethylamine) was added to this 250 ml beaker (excluding acrylate and 20 g fragrance.

The tetra-acrylate, PETA (4.88 g, pentaerythritol tetra acrylate) was mixed in 20 g of peppermint oil in another (100) ml beaker. In parallel the external (water) phase (206 g water, 32.8 g Poval (10%), 0.2 g Agitan 295) was prepared in a 500 ml beaker with an overhead stirrer.

The acrylate-oil mixture was added to the other part of the internal phase and mixed for 30 seconds. All of the mixed internal (oil) phase was then added to the external water phase and mixed for 1 minute and homogenized using an Ika mixer at 4000 rpm (homogenization) and then transferred to the prepared reaction flask. The reaction was run in the flask at 35° C. for 24 hours. Microcapsules with peppermint oil inside (17 wt % ˜100% efficiency) were made in a slurry.

### Example 26A: Microcapsules with Shea Butter (Ref 215-62-1)

A similar process as in Example 26 was applied to encapsulate shea butter using again TMPP (0.1 mol eq functional group), PTKHP (0.9 mol eq) and PETA Tetra-acrylate (1.0 mol eq). Spherical microcapsules with shea butter inside were formed. The shea butter was visibly released when crushed.

### Example 27: Microcapsules with Fragrance Sunburst Fresh R14-3913 Using a Difunctional Acrylate in the In-Situ Michael Addition Microencapsulation Process (Ref 210-66-1 BDDA (1.0 Mol Eq Functional Group), TMPP (0.1 Mol Eq), Pentaerythritol Hexakis(3-Mercaptopropionate) (PTHKMP, 0.9 Mol Eq)

An aqueous phase was prepared by mixing 32.8 g of 10% aqueous solution of Polyvinyl alcohol and 206 g of deionized water. 0.2 g of defoamer was also added.

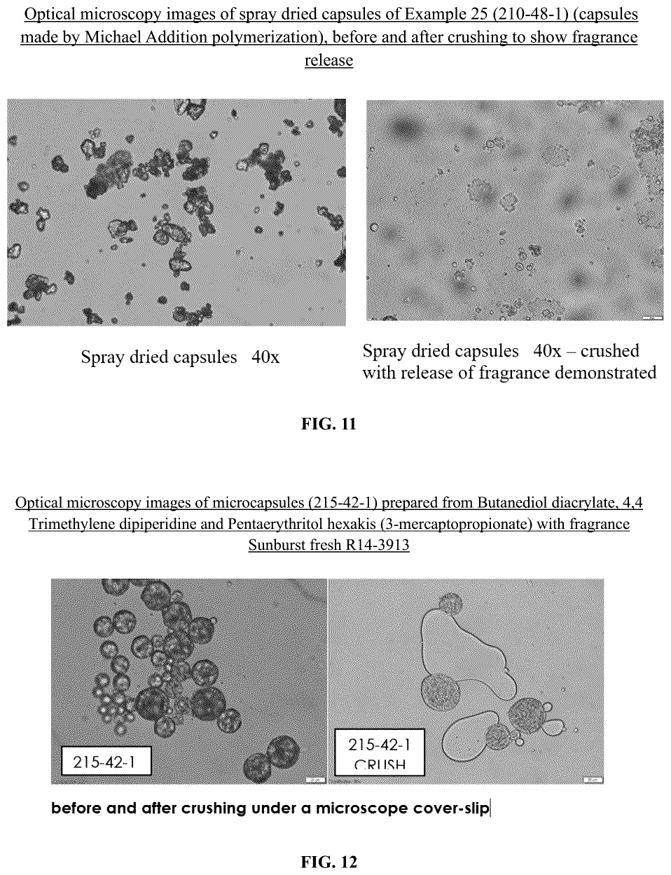

An oil phase was prepared by dissolving 0.58 g 4,4 trimethylene dipiperidine in 54 g Fragrance Sunburst fresh. 11 g of Propylene glycol dicaprylate/caprate was added followed by 0.36 g of Triethylamine and 6.19 g of Pentaerythritol hexakis(3-mercaptopropionate). 5.22 g of butanediol diacrylate was mixed into the oil phase. The oil phase was added to the aqueous phase under mechanical stirring to form a coarse emulsion. The course emulsion was homogenized using IKA magic lab homogenizer, 1 pass at 4000 rpm. The formed emulsion was transferred to a reactor pot and the emulsion was heated up to 35° C. The oil-in-water emulsion was then left to react for 24 hours to complete polymerization. The resulting microcapsule slurry was an aqueous slurry of microcapsules which were visible under a light microscope, as shown in FIG. 12.

### Example 28: Preparation of Microcapsules Having Polymer Shell Comprising Butanediol Diacrylate, 4,4 Trimethylene Dipiperidine and Pentaerythritol Hexakis (3-Mercaptopropionate) and Encapsulation of Home Care Fragrance Sunburst Fresh R14-3913 (Ref 215-42-1)

An aqueous phase was prepared by mixing 32.8 g of 10% aqueous solution of Polyvinyl alcohol and 206 g of deionized water. 0.2 g of defoamer was also added. An acrylate/amine Pre-polymer was prepared by dissolving 0.17 g Trimethylene Dipiperidine (TMPP) and 5.06 g Pentaerythritol tetraacrylate in 33.4 g Fragrance Sunburst fresh. An oil phase was prepared by dissolving 6.7 g of Pentaerythritol hexakis (3-mercaptopropionate) in 20 g Fragrance Sunburst fresh. 10.7 g of Propylene glycol dicaprylate/caprate was added followed by 0.36 g of Triethylamine. The oil phase was added to the pre-polymer under mechanical stirring to form the internal phase. The internal phase was added to the aqueous phase under mechanical stirring to form a coarse emulsion. The course emulsion was homogenized using IKA magic lab homogenizer, 1 pass at 4000 rpm. The formed emulsion was transferred to a reactor pot and the emulsion was heated up to 35° C. The oil-in-water emulsion was then left to react for 24 hours to complete polymerization. The resulting microcapsule slurry was an aqueous slurry of microcapsules which were visible under a light microscope. Results are provided in FIG. 12.

### Example 29: Coated/Multilayered Capsules

Samples (their slurries, as made) of the capsules of the invention (samples 215-09-1, 215-15-1 and 215-16-1) were filtered and encapsulated in a second coating of crosslinked sodium alginate. The filtered capsules were dispersed into a buffered solution of sodium alginate in water. That mixture was added slowly with stirring (via an addition funnel or syringe) into stirred solution of calcium chloride. Larger capsules than the original (‘visible beads’) were formed which were composed of the microcapsules of the invention surrounded or embedded in a crosslinked alginate coating or overlayer.

### Examples 30: Samples for Biodegradation Testing (Michael Addition Polymerizations)

Equivalent compositions (analogous shell materials) of the microcapsules described above could also be made, without fragrance, using dichloromethane as another lipophilic cargo, which was, for the purposes of testing, then subsequently evaporated to leave polymeric shell material only, for use in biodegradation testing.

### Example 31: (213-05-1/213-06-1: Encapsulated DCM Solvent (Subsequently Removed by Evaporation) for Biodegradation Testing of Polymeric Shell Material; Penta/Hexa Acrylate (1.0); Tetrathiol (0.9)—TMPP (0.1)

0.24 g (1.2 mmol) 4,4′trimethylenedipiperidine (TMPP) and 2.50 g (5.1 mmol) pentaerythritol tetrakis(3-mercaptopropionate, PTKMP) was added to a glass jar with 0.14 g (1.2 mmol) triethylamine. To this jar a magnetic stirrer bar was added along with 25.6 g dichloromethane. In parallel, an aqueous phase of 206 g (11.44 mol) water, 32.8 g of a 10% w/w solution of polyvinyl alcohol and 0.2 g Agitan 295 defoamer was added and stirred at 150 rpm using an overhead stirrer. A syringe was charged with 2 g (3.8 mmol) dipentaerythritol penta/hexaacrylate (DiPEHA) and this was added to the oil phase, stirring for 30 s until the acrylate monomer was well mixed. The oil phase was quickly added to the aqueous phase and allowed to stir for 5 minutes to form a coarse emulsion. This emulsion was homogenised with a IKA Magic LAB at 4000 rpm and charged into a 1 L resin pot with an anchor stirrer and an attached condenser. The reaction mixture was stirred at 150 rpm and 35 C for 24 hours. Following this, the mixture was transferred to a beaker with a magnetic stirred bar and allowed to stir in a fume hood for 72 hours to allow evaporation of the dichloromethane. No solvent was detected via GC following this. This dispersion was assessed for biodegradability via 301F, using an activated sludge inoculum.

This sample (prepared from penta/hexa acrylate and a tetra thiol) with dichloromethane cargo subsequently evaporated is similar to 210-26-1 (equivalent shell prepared with fragrance, described above, which showed a fragrance release/bloom after formulation into a representative fabric conditioner system), showed 21% biodegradation after 28 days in the OECD 301F test using activated sludge sourced from a local water treatment plant (Yorkshire Water) and 47% biodegradation after 60 days.

Other samples for biodegradation following the same approach for preparation with dichloromethane (DCM) as cargo and subsequently evaporated to leave residual shell material only, for biodegradation tests (OECD 301F protocols; activate sludge) were prepared. Their compositions are recorded in a tabulated summary below (Table 9) and example plots are shown in FIG. 17.

### Examples 32: Sensory and Other Fragrance Release Testing

Examples of microcapsules with fragrance inside prepared by in-situ oil in water Michael Addition polymerization displaying a fragrance bloom (triggered release (rubbing) via sensory panel testing) after formulating into a fabric conditioner base are summarized below. Examples of analogous shell wall preparations (DCM Method) for OECD 301F biodegradation testing are also summarized and where evidence of biodegradability was shown.

TABLE 9
Examples of microcapsules made via an in-situ oil-in-water Michael Addition Polymerization-
Encapsulation including analogous samples for biodegradation testing

| | | | Capsule Ref for | |
|---|---|---|---|---|
| Acrylate | Thiol | Amine | biodegradation | |
| (functionality; | (functionality; | (functionality; | tests (DCM | Equivalent Capsule |
| mol eq C═C) | mol eq SH) | mol eq NH) | cargo) | with fragrance |
| Penta/Hexa (5/6; 1.0) | Tetra (4; 0.9) | TMPP (2; 0.1) | 213-06-1 | 210-26-1 and 215-01-1; |
| Tetra (4; 1.0) | Hexa (6; 0.9) | TMPP (2; 0.1) | 215-19-1 | 215-15-1 |
| Penta/Hexa (5/6; 1.0) | Hexa (6; 0.9) | TMPP (2; 0.1) | 215-20-1 | 215-09-1 |
| Penta/Hexa (5/6; 1.0) | Tetra (4; 0.9) | TMPP (2; 0.1) | 215-21-1 | 215-38-1 |
| Penta/Hexa (5/6; 1.0) | Tetra (4; 1.0) | — | 215-22-1 | 215-12-1 |
| Penta/Hexa (5/6) | Hexa (6; 1.0) | — | 215-25-1 | 215-24-1 |
| Tetra (4; 1.0) | Hexa (6 ;1.0) | — | 215-26-1 | 215-16-1 |
| Tetra (4; 1.0) | Tetra (6; 0.9) | TMPP (2; 0.1) | 215-27-1 | 201-87-1; 201-873 |
| | | | | (87-1 + xanthan gum) |

Notes:
Tetra thiol = pentaerythritol tetrakis(3-mercaptopropionate (PTTKMP)
Hexa thiol = Pentaerythritol hexakis (3-mercaptopropionate) (PTHKMP)
Penta/hexa acrylate = dipentaerythritol penta/hexaacrylate (DiPEHA)
Tetra acrylate = Pentaerythritol tetraacrylate
Tri acrylate = Pentaerythritol triacrylate
TMPP = 4,4′trimethylenedipiperidine
TMPTA = trimethylolpropanetriacrylate

Test Procedures for Samples for Fragrance Bloom Testing (Fabric Conditioner Base):

Capsule slurries were tested in blind sensory evaluations (fragrance bloom tests) with a collection of people (minimum 2, typically 3-5). Note: Microcapsules produced typically contained ˜15-30 wt % fragrance encapsulated in the slurry—most used in the data reported were ˜17 wt %.

Typical Pre-Screening Sensory Test

A test mixture of the slurry, is prepared using 18 g of a fabric conditioner/softener formulation and an amount of slurry such that the fragrance loading in the test mixture is 0.1 g fragrance (based on the fragrance amount encapsulated in a slurry) and water added to make 20 g of test mixture.

In parallel a fabric wash mixture was prepared using each test mixture, each in a 2 litre beaker using an overhead stirrer at 250 rpm. This comprised 2 g of each test mixture above and 998 g of water (tap). Small squares (approx. 75 mm×75 mm; number is according to the number of people testing the samples of that slurry) of towel material were added to the beaker and stirred for 5 minutes after which they were removed and hung to dry in the air overnight, for 16 hours.

The next day another person or people (who did not make up the samples) smelt (sniffed) an untreated towel and then sniffed a sample of pure fragrance, and marked each in terms of an intensity number for these reference points, between 1 to 9 (9=highest fragrance intensity—as in neat fragrance typically). Randomly, samples prepared on towels as described were selected and sniffed and an intensity number recorded. Then the towel material is rubbed together for 5 seconds and sniffed again. The intensity after rubbing (post rub) was also recorded. This was repeated at random until all samples are tested as such by each panel member.

An average intensity is calculated for a before (pre-rub) and after (post rub) the rubbing for each sample. Example data are show further below.

Detailed Sensory Test: In another test method, a terg-o-tometer was used.

A prototype fabric softener/conditioner base formulation for such testing comprised of:

| Ingredient | Base fabric |
|---|---|
| Phase A | conditioner (wt. %) |
| Deionized water | 83.850 |
| Methyl bis[ethyl(tallowate)]-2-hydroxyethyl | 5.55 |
| ammonium methyl sulfate - Stepantex VT-90, | |
| Stepan Co. | |
| Poly(2-dimethylamino) ethyl methacrylate methyl | 0.5 |
| chloride quaternary salt | |
| chloromethylisothiazolinone (CMIT) and | 0.1 |
| methylisothiazolinone (MIT) - TroyGuard CM1.5, | |
| Troy corp. | |
| Total | 90.00 |

Prototype fabric softener/conditioner with a hole of 10% to accommodate for other ingredients to be added later. Prepare phase B containing 0.5% active neat fragrance or fragrance encapsulate by pre-mixing with an equal active amount of emulsifier such as Tomadol 1-73B to be added to the fabric conditioner base phase A. The conditioner is then balanced with deionized water. Finally, pH of the prepared fabric conditioner base is adjusted to pH 2.5-3.5 with weak acid, if needed. The Brookfield viscosity of the prepared base varies between 100-600 cP, depending on fragrance encapsulate test material. All capsules tested were observed to be stable in the formulation.

Terg-o-Tometer Test Methodology
Water hardness: 200 ppm (3Ca²⁺/1Mg²⁺); Temperature: 100° F.; Fabric conditioner: 2 g/L
Test fabrics: Pre-conditioned cotton terry towel 12×12 inches cut into 3×3 inches squares, use 10 pieces per 1 L wash soln.
Rinse time: 5-min. at 100 rpm agitation. After rinse, squeeze excess water; Dry: Airline dry overnight.

Sensory panel evaluation: Expert panelist are asked to smell references before scoring samples for fragrance intensity score=1 least intense and score=9/10 most intense. Afterwards, panelist is given one treated towel sample blindly to score for relative fragrance intensity before and after rubbing.

Examples of data from fragrance bloom sensory panel release tests after formulating into a formulation representative of a liquid laundry fabric conditioner and applying to fabric (before and after rubbing) are given below (FIGS. 13-16).

### Example 33: Sensory/Applications Tests

Two samples of microcapsules prepared according to the procedures above were assessed in various formulations for fragrance release. They were:
215-15-1; Tetraacrylate-PTHKMP Hexathiol-TMPP (1: 0.9: 0.1): capsules 24% solids in water; fragrance: 17 wt % in slurry
210-50-1; DiPETA Penta/Hexaacrylate-PTHKMP Hexathiol-TMPP—CEA (carboxyethyl acrylate) (1: 0.9: 0.1: 0.1): capsules 24% solids in water; fragrance: 17 wt % in slurry

### Example 33A: Fragrance Encapsulates in a Combing Cream Formulation

Two types of capsules (codes 210-50-1 and 215-15-10) were post added in the following formulation:

| Ingredient | Amount |
|---|---|
| Aqua (water) | 62.490 |
| Methylparaben | 0.100 |
| Polyquaternium-37 (and) Propylene Glycol | 2.725 |
| Dicaprylate/Dicaprate (and) PPG-1 Trideceth-6 | |
| Guar Hydroxypropyltrimonium Chloride | 30.000 |
| (and) Acrylates Copolymer | |
| Cetrimonium Chloride | 0.670 |
| Dimethicone | 1.000 |
| Cyclo pentasiloxane | 1.000 |
| Phenoxyethanol (and) Caprylyl Glycol | 1.000 |
| Fragrance/Parfum | 0.300 |
| Water (and) Green 8 (CI 59040) | 0.700 |
| Green 5 (CI 61570) | 0.015 |

Each capsule was dosed in at 0.5% and 1% per weight and samples coded for blind assessments by three untrained panellists. Fragrance assessment was conducted on hair tress weighing 5 grams, measuring 10″ in length of Caucasian hair bleached once. 0.5 grams of each sample was weighed, spread over a dry hair tress and massaged in gently for 20 seconds. Samples were left to rest for 15 minutes. Three untrained panellists were asked to perform a pair comparison test, answering the question ‘which sample smells the strongest of the two?’. They were asked to do the fragrance assessment before and after combing through 3 times with a fine-tooth comb. All samples containing fragrance capsules were noted as ‘smelling stronger’ after combing by all panellists over the control. The capsules were observed to be stable in the formulation.

### Example 34B: Fragrance Encapsulates in Styling Gel Formulation

Two types of capsules (codes 210-50-1 and 215-15-10) were post added in the following formulation:

| Ingredient | Amount |
|---|---|
| Aqua (water) | 77.110 |
| Tetrasodium EDTA | 0.100 |
| Carbomer | 0.500 |
| Acrylates Copolymer (and) Water (aqua) | 3.330 |
| Aqua (water) | 5.000 |
| Isobutylene/Ethylmaleimide/Hydroxyethyl | 2.500 |
| Maleimide Copolymer | |
| Aminomethyl Propanol | 0.960 |
| Sorbitol | 3.000 |
| Glycerin | 2.000 |
| Aqua (water) | 5.000 |
| Oleth-5 Phosphate | 0.150 |
| Fragrance/Parfum | 0.050 |
| Phenoxyethanol (and) Caprylyl Glycol | 0.300 |

Each capsule was dosed in at 0.5% and 1% per weight and samples coded for blind assessments by three untrained panellists. The fragrance assessment was conducted on hair tress weighing 5 grams, measuring 10″ in length of Caucasian hair bleached once. 0.5 grams of each sample was weighted, spread over a dry hair tress and massaged in gently for 20 seconds. Samples were left to rest for 15 minutes. Three untrained panellists were asked to perform a pair comparison test, answering the question ‘which sample smells the strongest of the two?’. They were asked to do the fragrance assessment before and after combing through 3 times with a fine-tooth comb. Before combing, all panellists agreed that all samples were very similar, with barely any smell. All samples containing fragrance capsules were noted as ‘smelling stronger’ by all panellists over the control, after combing. Both capsules showed a dose response. The capsules were observed to be stable in the formulation.

### Example 34C: Fragrance Encapsulates in a Heavy-Duty Cleaner Formulation

Two types of capsules (codes 210-50-1 and 215-15-10) were post added in the following formulation:

| Ingredient | Amount |
|---|---|
| Aqua (water) | 94.155 |
| Sodium Hydroxide | 0.040 |
| Acrylic acid/vinyl pyrrolidone crosspolymer | 0.140 |
| C6-12 Alcohols Ethoxylated Propoxylated | 5.700 |

Each capsule was dosed in at 0.5% and 1% per weight and samples coded for blind assessments by three untrained panellists. The fragrance assessment was conducted on glass plates. 0.5 grams of each sample was weighed and pipetted onto the glass surface. Samples were spread around the plate by tilting the plate to minimize mechanical manipulation. Samples were left to rest for 20 minutes in a fume hood until dried. Three untrained panellists were asked to perform a pair comparison test, answering the question ‘which sample smells the strongest of the two?’. They were asked to do the fragrance assessment before and after rubbing through 3 times, with a gloved finger. The same scientist was performing the rubbing for all. Before rubbing, all panellists agreed that all samples were very similar, with barely any smell. All samples containing fragrance capsules were noted as ‘smelling stronger’ by all panellists over the control after rubbing. The capsules were observed to be stable in the formulation.

### Example 34D: Fragrance Encapsulates in an all-Purpose Cleaner Formulation

Two types of capsules (codes 210-50-1 and 215-15-10) were post added in the following formulation:

| Ingredient | Amount |
|---|---|
| Aqua (water) | 98.605 |
| Sodium Hydroxide | 0.040 |
| Acrylic acid/vinyl pyrrolidone crosspolymer | 0.140 |
| C6-12 Alcohols Ethoxylated Propoxylated | 1.250 |
| Seed Oil Alcohol Ethoxylate 9EO | |

Each capsule was dosed in at 0.5% and 1% per weight and samples coded for blind assessments by three untrained panellists. The fragrance assessment was conducted on glass plates. 0.5 grams of each sample was weighed and pipetted onto the glass surface. Samples were spread around the plate by tilting the plate to minimize mechanical manipulation. Samples were left to rest for 20 minutes in a fume hood until dried. Three untrained panellists were asked to perform a pair comparison test, answering the question ‘which sample smells the strongest of the two?’. They were asked to do the fragrance assessment before and after rubbing through 3 times, with a gloved finger. The same scientist was performing the rubbing for all. Before rubbing, all panellists agreed that all samples were very similar, with barely any smell. All samples containing fragrance capsules were noted as ‘smelling stronger’ by all panellists over the control after rubbing. The capsules were observed to be stable in the formulation.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

1. A microcapsule comprising:
(i) a lipophilic core; and
(ii) a polymeric microcapsule shell;
wherein, the polymeric microcapsule shell comprises a polymer or a crosslinked polymer of an aliphatic polyester or a poly-β-amino-ester or a poly-β-thio-ester or their co-polymers or ter-polymers or mixtures thereof; and
wherein, the microcapsule is storage stable and its polymeric shell is biodegradable.

2. The microcapsule according to claim 1, wherein the microcapsule shell comprises a branched or crosslinked polymer derived from an aliphatic polyester prepolymer selected from aliphatic polyester comprising at least one reactive unsaturation functionality present either at a chain end or distributed along the chain.

3. The microcapsule according to claim 1, wherein the aliphatic polyester comprises a crystalline structure.

4. The microcapsule according to claim 1, wherein the aliphatic polyester is derived from at least one diacid, diester, diacyl chloride, or anhydride comprising C₂-Cao aliphatic chain or branched C₂-C₂₀ aliphatic chain or combinations thereof; and at least one diol comprising C₂-C₂₀ aliphatic chain or branched C₂-C₂₀ aliphatic chain or combinations thereof.

5. The microcapsule according to claim 1, wherein the aliphatic polyester is derived from at least one diacid selected from the group consisting of succinic acid, propanedioic acid, butanedioic acid, hexanedioic acid, octanedioic acid, decanedioic acid, sebacic acid, dodecanedioic acid, octenyl succinic acid, itaconic acid, maleic acid and dodecenylsuccinic acid, or at least one anhydride selected from the group consisting of succinic anhydride, dodecenylsuccinic anhydride and octenyl succinic anhydride; and at least one diol selected from the group consisting of ethylene glycol, propylene glycol, butanediol, pentanediol, hexanediol, octane diol, decanediol, cyclohexane dimethanol, isosorbide, neopentyl glycol, ethyl hexane diol and dodecanediol.

6. The microcapsule according to claim 1, wherein the aliphatic polyester is a polymer derived from at least one lactide and at least one glycolide.

7. The microcapsule according to claim 6, wherein the aliphatic polyester is coupled with an attached oil solubilizing oligo ester or polyester chain.

8. The microcapsule according to claim 7, wherein the oil solubilizing oligo ester or polyester chain is polyester comprising an alkyl side chain of C₂-C₂₀ aliphatic chain or branched C₂-C₂₀ aliphatic chain or combinations thereof or is an oligo- or poly-caprolactone.

9. The microcapsule according to claim 2, wherein the reactive unsaturation functionality is selected from the group consisting of acrylate, methacrylate, itaconate, citraconate, maleate, fumarate, crotonate and combinations thereof.

10. The microcapsule according to claim 2, wherein aliphatic polyester prepolymer with reactive unsaturation is an itaconate containing polyester or is (i) an acrylate, diacrylate, or multifunctional acrylate of a polyester; (ii) an acrylate, diacrylate, or multifunctional acrylate of an epoxide; (iii) an acrylate, diacrylate, or multifunctional acrylate of an urethane; or (iv) an acrylate, diacrylate, or multifunctional acrylate of a polyether; or combinations thereof.

11. The microcapsule according to claim 1, wherein the polymer or crosslinked polymer is a poly-β-amino-ester or a poly-β-thio-ester or any combination thereof, derived from a Michael or conjugate addition reaction of a donor and acceptor, wherein the donor or acceptor has a reactive functionality of at least two or at least three.

12. The microcapsule according to claim 1, wherein the crosslinked polymer is a poly-β-amino-ester or a poly-β-thio-ester or combination thereof is derived from a Michael or conjugate addition reaction of: (i) at least one multifunctional donor having a reactive functionality of at least three; and (ii) at least one multifunctional acceptor having a reactive functionality of at least three.

13. The microcapsules according to claim 11, wherein the donor is an amine or a thiol or mixture of amine and thiol.

14. The microcapsule according to claim 13, wherein the donor is a mixture of at least one difunctional thiol or multifunctional thiol and at least one difunctional amine or multifunctional amine.

15. The microcapsule according to claim 13, wherein the amine is a difunctional primary amine, a multifunctional primary amine, a difunctional secondary amine or a multifunctional secondary amine.

16. The microcapsule according to claim 13, wherein the amine comprises

17. The microcapsule according to claim 13, wherein the difunctional amine or multi-functional amine is selected from the group consisting of 4,4′trimethylenepiperidine (TMPP), isophorone diamine, bis-(aminomethyl)cyclohexane, cyclohexane diamine, piperazine, aminoethylpiperazine, bis-amino-norbornane, diethylene triamine, diethylene diamine, tetraethylene pentamine, hexamethylene diamine, diamino propane, diamino butane, decane diamine, dodecane diamine, and polyethyleneimine.

18. The microcapsule according to claim 13, wherein the donor is a mixture of one or more thiol and one or more amine, and the amine functional group (NH) is present in an amount of about ≤50 or ≤25 or ≤20% of total or combined mole equivalents of thiol and amine functional groups (SH and NH).

19. The microcapsule according to claim 11, wherein the crosslinked polymer comprises poly β-amino ester, poly-β thio ester or copolymers thereof.

20. The microcapsule according to claim 12, wherein the multifunctional donor and multifunctional acceptor each comprise at least one tri-functional, tetra-functional, penta-functional or hexa functional reactive groups.

21. The microcapsule according to claim 11, wherein the acceptor is selected from the group consisting of an acrylate, methacrylate, maleate, fumarate, itaconate, malonate, crotonate, citraconate, maleimide and mixtures thereof.

22. The microcapsule according to claim 11, wherein the acceptor is selected from the group consisting of trimethylol propane triacrylate, pentaerythritol triacrylate, pentaerythritol tetra acrylate, dipentaerythritol penta acrylate, dipentaerythritol hexa acrylate, or is an itaconate containing polyester, or an acrylate, diacrylate, or multifunctional acrylate of a polyester; (i) acrylate, diacrylate, or multifunctional acrylate of an epoxide; (ii) acrylate, diacrylate, or multifunctional acrylate of an urethane; or (iii) acrylate, diacrylate, or multifunctional acrylate of a polyether; or combinations thereof.

23. The microcapsule according to claim 11, wherein the polymeric shell is derived from a donor-acceptor combination selected from the group comprising:
(i) a trifunctional thiol, tetrafunctional thiol, pentafunctional thiol or hexafunctional thiol; and
(ii) a trifunctional acrylate, tetrafunctional acrylate, pentafunctional acrylate or hexafunctional acrylate.

24. The microcapsule according to claim 23, wherein the donor-acceptor combination further comprises difunctional amine, trifunctional amine, tetrafunctional amine, pentafunctional amine or hexafunctional amine.

25. The microcapsule according to claim 12, wherein the acceptor comprises difunctional acrylate.

26. The microcapsule according to claim 19, wherein crosslinked polymer comprises combination or copolymer of β-amino ester and β-thio ester, wherein the β-amino ester is present in an amount of about ≤50 or ≤25 or ≤20 mol equivalent % of total mole equivalents of thio-ester and amino-ester.

27. The microcapsule according to claim 1, wherein the lipophilic core is selected from the group comprising agrochemicals, aliphatic esters, anti-microbial agents, anti-fungal, anti-fouling agents, antioxidants, anti-viral agents, biocides, catalysts, cosmetic actives, dyes, colorants, detergents, edible oils, emollient oils, essential oils, fats, fatty acids, fatty acid esters, food additives, flavors, fragrances, hair care actives, halogenated compounds, hydrocarbons, insecticides, insect repellants, lipids, lipophilic scale inhibitors, mineral oil, oral care actives, organic solvents, organic esters, chlorinated solvents, pesticides, perfumes, preservatives, skin care actives, UV absorbers, vegetable oils and combinations thereof.

28. The microcapsule according to claim 27, wherein the core is fragrance, perfume or an essential oil.

29. The microcapsule according to claim 1, wherein the microcapsule is used in a consumer care compositions selected from the group comprising laundry care compositions, oral care compositions, hair care compositions, skin care compositions, cosmetic care compositions, home care and cleaning compositions.

30. The microcapsule according to claim 29, wherein the microcapsule is used in a fabric conditioner composition or a laundry detergent composition.

31. The microcapsule according to claim 1, wherein the polymeric microcapsule shell is biodegradable in an aquatic medium or solid medium or is compostable.

32. The microcapsule according to claim 31, wherein the aquatic or solid medium is selected from group consisting of activated sludge, secondary effluent, river water, surface water, fresh water, sea water, soil and compost.

33. The microcapsule according to claim 32, wherein the polymeric microcapsule shell material shows a biodegradation rate of at least 20% in an aquatic medium when measured by an OECD Test method 301, 302 or 306.

34. The microcapsule according to claim 32, wherein the polymeric microcapsule shell material shows evidence of biodegradation within 120 days or within 60 days or within 40 days or within 28 days.

35. The microcapsule according to claim 1, wherein the microcapsule is stable as a core-shell capsule in an aqueous slurry, in a water-based formulation or in a solvent-based formulation.

36. The microcapsule according to claim 1, wherein the microcapsule is storage stable as a core-shell capsule in solid formulation or in printed product.

37. The microcapsule according to claim 35, wherein the formulation is selected from the group consisting of laundry detergent, fabric softener, fabric conditioner, shampoo, hair conditioner, liquid soap, solid soap, skin deodorant, skin moisturizer, skin conditioner, hair or skin protectant, cleanser, sanitizer, cleaning fluid, dishwashing washing fluid or tablet, washing powder or tablet or liquid, and cosmetic formulation.

38. The microcapsule according to claim 35, wherein the formulation is in the pH range of about 3-11, 3 to 6, 6 to 8, or 8 to 11.

39. The microcapsule according to claim 1, wherein the microcapsule is part of a double layered microcapsule, a multi-layered microcapsule or an overcoated microcapsule.

40. The microcapsule according to claim 39, wherein the double layered or multilayered or an overcoated microcapsule also comprises a hydrogel or a crosslinked alginate.

41. The microcapsule according to claim 1, wherein the microcapsule has an average diameter of about 100 nm to 100 μm or of about 1 μm to 100 μm.

42. A method for preparing a microcapsule of claim 1, the method comprising:
(a) preparing an oil-in-water emulsion of (i) an oil phase comprising a polymer or a prepolymer, and at least one lipophilic core; and (ii) a water phase comprising at least one stabilizer or emulsifier,
(b) optionally adding at least one catalyst, at least one diluent or at least one initiator to the oil phase;
(c) optionally heating the oil-in-water emulsion with stirring to a temperature between 25° C. and 100° C.;
(d) forming the polymeric microcapsule shell either by cooling or by an in-situ oil-in-water reaction of the polymer or prepolymer; and
(e) obtaining the lipophilic core encapsulated in a polymeric microcapsule shell, wherein, the polymer or prepolymer formed is an aliphatic polyester or a poly-β-amino ester or a poly-β-thio ester or their co-polymers or ter-polymers or combinations thereof.

43. The method according to claim 42, wherein the polymer or prepolymer is an aliphatic polyester.

44. The method according to claim 43, wherein the polymeric shell formed comprises an aliphatic polyester with a crystalline structure.

45. The method according to claim 43, wherein the polymeric shell formed comprises a crosslinked aliphatic polyester.

46. The method according to claim 43, wherein the microcapsule shell formed comprises a branched or crosslinked polymer derived from an aliphatic polyester prepolymer comprising at least one reactive unsaturation functionality present either at a chain end or distributed along the chain.

47. The method according to 43, wherein the aliphatic polyester is derived from at least one diacid, diester, diacyl chloride, or anhydride comprising C₂-C₂₀ aliphatic chain or branched C₂-C₂₀ aliphatic chain or combinations thereof; and at least one diol comprising C₂-C₂₀ aliphatic chain or branched C₂-C₂₀ aliphatic chain or combinations thereof.

48. The method according to claim 47, wherein the aliphatic polyester is derived from at least one diacid selected from the group consisting of succinic acid, propanedioic acid, butanedioic acid, hexanedioic acid, octanedioic acid, decanedioic acid, sebacic acid, dodecanedioic acid, dodecenylsuccinic acid, octenyl succinic acid, itaconic acid and maleic acid; at least one anhydride selected from the group consisting of succinic anhydride, dodecenylsuccinic anhydride and octenyl succinic anhydride, diacyl chlorides or anhydrides; and at least one diol selected from ethylene glycol, propylene glycol, butanediol, pentanediol, hexanediol, octane diol, decanediol, cyclohexane dimethanol, isosorbide, neopentyl glycol, ethyl hexane diol and dodecanediol.

49. The method according to claim 43, wherein the aliphatic polyester is a polymer derived from at least one lactide, glycolide or caprolactone functionality.

50. The method according to claim 49, wherein the aliphatic polyester is a polymer derived by ring opening polymerization of a lactide or a glycolide or a combination of lactide and glycolide, coupled with an attached oil solubilizing or solvent solubilizing oligo ester or polyester chain used as a co-initiator or linked through copolymerization or a reactive coupling.

51. The method according to claim 46, wherein the reactive unsaturation functionality is selected from the group consisting of acrylate, methacrylate, itaconate, citraconate, maleate, fumarate, crotonate and combinations thereof.

52. The method according to claim 46, wherein the aliphatic polyester prepolymer with reactive unsaturation is an itaconate containing polyester or is (i) an acrylate, diacrylate, or multifunctional acrylate of a polyester; (ii) an acrylate, diacrylate, or multifunctional acrylate of an epoxide; (iii) an acrylate, diacrylate, or multifunctional acrylate of an urethane; or (iv) an acrylate, diacrylate, or multifunctional acrylate of a polyether; or combinations thereof.

53. The method according to claim 42, wherein the water phase or oil phase comprises a radical initiator system selected from the group consisting of a peroxide based, an azo based or a redox based initiator.

54. The method according to claim 42, wherein the prepolymer contains unsaturated groups at a chain end or distributed along the chain and the in-situ reaction to form the polymeric shell includes reaction of the prepolymer containing unsaturated groups via (i) a chain extension, (ii) branching or (iii) crosslinking reaction.

55. The method according to claim 42, wherein the in-situ reaction is a self-reaction or a radical polymerization reaction at a temperature ≤100° C.

56. The method according to claim 42, wherein the prepolymer contains conjugated unsaturated groups at a chain end or distributed along the chain and the in-situ reaction to form the polymeric shell includes Michael Addition reaction of the prepolymer containing conjugated unsaturation with a difunctional amine or multifunctional amine or a difunctional thiol or multifunctional thiol via (i) a chain extension, (ii) branching or (iii) crosslinking.

57. The method according to claim 42, wherein the prepolymer contains reactive acid or anhydride groups at a chain end or distributed along the chain and the in-situ reaction includes reaction of at least one acid or anhydride group of the prepolymer with at least one difunctional epoxide or multifunctional epoxide or difunctional amine or multifunctional amine via (i) a chain extension, (ii) branching or (iii) crosslinking.

58. The method according to claim 42, wherein the oil phase optionally comprises at least one diluent or solvent.

59. The method according to claim 58, wherein the diluent or solvent is selected from a group consisting of hydrocarbon oil, alkanes, an ester oils, a fatty acid esters, an aliphatic esters, and alkylene carbonates.

60. The method according to claim 42, wherein the oil phase is homogeneous and is prepared with optional heating up to a temperature of about 100° C.

61. The method according to claim 42, wherein the water phase optionally further comprises at least one additive selected from the group consisting of surfactants, defoamers, rheology modifiers, thickeners, partitioning inhibitors, radical inhibitors, catalysts, radical initiators or combinations thereof.

62. The method according to claim 42, wherein the
stabilizer or emulsifier is selected from the group consisting of polyvinyl alcohol, hydroxyethyl cellulose, and polyvinylpyrrolidone; and
defoamer is selected from the group consisting of liquid hydrocarbons, oils, hydrophobic silicas, fatty acids, alkoxylated compounds, polyethers, polyalkylene glycols, and nonionic emulsifiers.

63. A method for preparing microcapsule of claim 1, the method comprising:
(a) preparing an oil-in-water emulsion of (i) an oil phase comprising monomeric reactants and at least one lipophilic core; and (ii) a water phase comprising at least one stabilizer or emulsifier,
(b) optionally adding at least one catalyst or at least one initiator to the oil phase or water phase,
(c) forming the polymeric microcapsule shell wall by an in-situ oil-in-water polymerization reaction of the monomeric reactants, and
(d) obtaining the lipophilic core encapsulated in a polymeric microcapsule shell.

64. The method according to claim 63, wherein the oil phase optionally comprises at least one diluent or solvent.

65. The method according to claim 64, wherein the diluent or solvent is selected from the group consisting of hydrocarbon oils, alkanes, ester oils, fatty acid esters, aliphatic esters, and alkylene carbonates.

66. The method according to claim 63, wherein the oil phase is homogeneous and is prepared by optionally heating up to a temperature of about ≤100° C. or ≤80° C. or ≤60° C.

67. The method according to claim 63, wherein the oil in water emulsion is prepared with or without application of heat.

68. The method according to claim 63, wherein the in-situ polymerization includes polycondensation or esterification reaction of monomeric reactants to form a polymeric shell comprising an aliphatic polyester.

69. The method according to claim 68, wherein the monomeric reactants are (a) at least one difunctional or multifunctional acid, difunctional or multifunctional acyl chloride, difunctional or multifunctional ester or a difunctional or multifunctional anhydride; and (b) at least one difunctional or multifunctional alcohol or a difunctional or multifunctional polyol.

70. The method according to claim 68, wherein the in-situ polycondensation reaction of the monomeric reactants is carried out at a temperature at or ≤100° C. or ≤95° C. or ≤80° C. to form the aliphatic polyester polymeric shell.

71. The method according to claim 68, wherein the polymeric shell formed comprises an aliphatic polyester with a crystalline structure.

72. The method according to claim 68, wherein the monomeric reactants forming the aliphatic polyester are derived from
at least one diacid, diester, diacyl chloride, or anhydride comprising C₂-C₂₀ aliphatic chain or branched C₂-C₂₀ aliphatic chain or combinations thereof, and
at least one diol comprising C₂-C₂₀ aliphatic chain or branched C₂-C₂₀ aliphatic chain or combinations thereof.

73. The method according to claim 68, wherein the aliphatic polyester derived from at least one diacid selected from the group consisting of succinic acid, propanedioic acid, butanedioic acid, hexanedioic acid, octanedioic acid, decanedioic acid, sebacic acid, dodecanedioic acid, dodecenylsuccinic acid, octenyl succinic acid, itaconic acid and maleic acid; at least one diester is selected from the group consisting of; at least one anhydride is selected from the group consisting of succinic anhydride, dodecenylsuccinic anhydride, and octenyl succinic anhydride; and at least one diol is selected from the group consisting of ethylene glycol, propylene glycol, butanediol, pentane diol, hexanediol, octane diol, decanediol, cyclo hexane dimethanol, isosorbide, neopentyl glycol, ethyl hexane diol and dodecanediol.

74. The method according to claim 68, wherein the catalyst is a sulfonic acid, phosphoric acid, tin octanoate, tin hexanoate, stannic acid or a stannic acid, tin oxide or tin based compound or is a lipase or other enzyme.

75. The method according to claim 63 wherein the in-situ polymerization reaction is a Michael or conjugate Addition reaction of donor and acceptor reactants to form a polymeric shell comprising β-amino ester and/or β-thio ester functionalities.

76. The method according to claim 75, wherein the monomeric reactants comprise (i) at least one difunctional thiol, multifunctional thiol, difunctional amine or multifunctional amine donor and (ii) at least one difunctional or multifunctional Michael acceptor.

77. The method according to claim 76, wherein the reactants comprise (i) at least one multifunctional donor having a reactive functionality of three or more; and (ii) at least one multifunctional acceptor having a reactive functionality of three or more.

78. The method according to claim 77, wherein the donor is a mixture of at least one thiol and at least one amine.

79. The method according to claim 76, wherein the amine is a difunctional or multifunctional diamine, a difunctional or multifunctional primary amine or a difunctional or multifunctional secondary amine.

80. The method according to claim 79, wherein the amine comprises a C₂-C₂₀ aliphatic chain, C₄-C₇ cyclic or a C₄-C₇ heterocyclic ring.

81. The method according to claim 80, wherein the amine is selected from the group consisting of trimethylenepiperidine (TMPP), isophorone diamine, bis-(aminomethyl)cy clohexane, cyclohexane diamine, piperazine, aminoethylpiperazine, bis-amino norbornane, diethylene triamine, diethylene diamine, tetraethylene penta amine, hexamethylene diamine, diamino decane, diamino dodecane, and polyethyleneimine.

82. The method according to claim 78, wherein the amine functional group (NH) is present in an amount of about ≤50 or ≤25 or ≤20 mol equivalent % of the total donor functional group.

83. The method according to claim 78, wherein the amine is incorporated via a pre-reaction before forming the oil-in-water emulsion with all or part of the multifunctional acceptor.

84. The method according to claim 75, wherein the method further comprises a water soluble or an oil soluble monofunctional Michael acceptor.

85. The method according to claim 75, wherein the method further comprises a radical initiator system added to water phase, oil phase or both phases at the start or part way through or near completion of the in-situ reaction.

86. The method according to claim 75, wherein the method further comprises a polymer added as powder or solution to either water or oil phase and wherein, the polymer is selected from a group consisting of an aliphatic polyester, chitosan, cellulose, cellulose based compounds and a protein.

87. The method according to claim 75, wherein the in-situ reaction is between (i) a tri thiol, a tetra thiol, a penta thiol, or a hexa thiol; and (ii) a tri acrylate, a tetra acrylate, a penta acrylate, or a hexa acrylate.

88. The method according to claim 75, wherein the
Michael acceptor is selected from the group consisting of trimethylol propane triacrylate, pentaerythritol triacrylate, pentaerythritol tetra-acrylate, dipentaerythritol penta acrylate, dipentaerythritol hexa acrylate, or
Michael acceptor is selected from the group consisting of an itaconate containing polyester or (i) an acrylate, diacrylate, or multifunctional acrylate of a polyester; (ii) an acrylate, diacrylate, or multifunctional acrylate of an epoxide; (iii) an acrylate, diacrylate, or multifunctional acrylate of an urethane; or (iv) an acrylate, diacrylate, or multifunctional acrylate of a polyether; or combinations thereof.

89. The method according to claim 75, wherein the in-situ reaction to form the polymeric shell is an addition reaction at a temperature of ≤80° C., or ≤60° C., or ≤50° C.

90. The method according to claim 63, wherein the water phase optionally comprises at least one additive selected from the group consisting of surfactants, defoamers, rheology modifiers, thickeners, partitioning inhibitors, radical inhibitors, catalysts and radical initiators.

91. A method for preparing microcapsules of claim 1, consisting of β-thio ester and β-amino ester functionalities, the method comprising:
(a) pre-reacting a difunctional or multifunctional amine with difunctional or multifunctional acrylate;
(b) preparing an oil-in-water emulsion of (i) an oil phase comprising the resultant or product of (a) and any remaining acceptor, mixed with a difunctional or multi-functional thiol, and at least one lipophilic core, optionally with a diluent and (ii) a water phase comprising at least one stabilizer or emulsifier;
(c) optionally adding at least one catalyst to the oil phase or water phase,
(d) forming the polymeric microcapsule shell wall by an in-situ oil-in-water Michael addition polymerization reaction of the donor and acceptor reactants, and
(e) obtaining the lipophilic core encapsulated in a polymeric microcapsule shell.

92. The method according to claim 84, wherein the monofunctional acceptor is acrylic acid, methacrylic acid, carboxyethyl acrylate, hydroxyalkyl acrylate or hydroxyalkyl methacrylate.

93. The method according to claim 91, wherein the acceptor is a multifunctional acrylate, methacrylate, maleate, fumarate, itaconate, malonate, crotonate, citraconate, maleimide or mixtures thereof.

94. The method according to claim 91, wherein the catalyst is a tertiary amine.
